(19) United States
(12) Patent Application Publication
(10) Pub. No.: US 2022/0090047 A1
(43) Pub. Date: Mar. 24, 2022

(54) GENETIC MODIFICATION OF THE HYDROXYACID OXIDASE 1 GENE FOR TREATMENT OF PRIMARY HYPEROXALURIA

(71) Applicant: Precision BioSciences, Inc., Durham, NC (US)

(72) Inventors: Roshni Davey, Raleigh, NC (US); Derek Jantz, Durham, NC (US); James Jefferson Smith, Morrisville, NC (US); Gary Owens, Durham, NC (US)

(73) Assignee: Precision BioSciences, Inc., Durham, NC (US)

(21) Appl. No.: 17/415,626

(22) PCT Filed: Dec. 20, 2019
(86) PCT No.: PCT/US2019/068186

§ 371 Date: Jun. 17, 2021

Related U.S. Application Data
(60) Provisional application No. 62/833,574, filed on Apr. 12, 2019.
(60) Provisional application No. 62/783,969, filed on Dec. 21, 2018.

(51) Int. Cl. C12N 15/10; C12N 9/16; A61P 1/16; A61K 38/46; C12N 15/86

(52) U.S. Cl. CPC C12N 15/102; C12N 9/16; C12N 15/86; A61K 38/465; A61P 1/16

(57) ABSTRACT

Disclosed are engineered nucleases that bind and cleave a recognition sequence within a hydroxyacid oxidase 1 (HAO1) gene. The present invention also encompasses methods of using such engineered nucleases to make genetically-modified cells. Further, the invention encompasses pharmaceutical compositions comprising engineered nuclease proteins or nucleic acids encoding engineered nucleases of the invention, and the use of such compositions for treatment of primary hyperoxaluria type I.

### FIELD OF THE INVENTION

The invention relates to the field of molecular biology and recombinant nucleic acid technology. In particular, the invention relates to engineered nucleases having specificity for a recognition sequence within a hydroxyacid oxidase 1 (HAO1) gene, and particularly within or adjacent to exon 8 of the HAO1 gene. Such engineered nucleases are useful in methods for treating primary hyperoxaluria.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 20, 2019 is named P109070030WO-SEQ-MJT and is 131,786 bytes in size.

### BACKGROUND OF THE INVENTION

Primary hyperoxaluria Type 1 (“PH1”) is a rare autosomal recessive disorder, caused by a mutation in the AGXT gene. The disorder results in deficiency of the liver-specific enzyme alanine:glyoxylate aminotransferase (AGT), encoded by AGXT. AGT is responsible for conversion of glyoxylate to glycine in the liver. Absence or mutation of this protein results in overproduction and excessive urinary excretion of oxalate, causing recurrent urolithiasis (i.e., kidney stones) and nephrocalcinosis (i.e., calcium oxalate deposits in the kidneys). As glomerular filtration rate declines due to progressive renal involvement, oxalate accumulates leading to systemic oxalosis. The diagnosis is based on clinical and sonographic findings, urine oxalate assessment, enzymology and/or DNA analysis. While early conservative treatment has aimed to maintain renal function, in chronic kidney disease Stages 4 and 5, the best outcomes to date have been achieved with combined liver-kidney transplantation (Cochat et al. Nephrol Dial Transplant 27: 1729-36). However, no approved therapeutics exist for treatment of PHL

PH1 is the most common form of primary hyperoxaluria and has an estimated prevalence of 1 to 3 cases in 1 million in Europe and approximately 32 cases per 1,000,000 in the Middle East, with symptoms appearing before four years of age in half of the patients. It accounts for 1 to 2% of cases of pediatric end-stage renal disease (ESRD), according to registries from Europe, the United States, and Japan (Harambat et al. Clin J Am Soc Nephrol 7: 458-65).

Hydroxyacid oxidase 1 (HAO1), which is also referred to as glycolate oxidase, is the enzyme responsible for converting glycolate to glyoxylate in the mitochondrial/peroxisomal glycine metabolism pathway in the liver and pancreas. When AGXT is incapable of converting glyoxylate to glycine, excess glyoxylate is converted in the cytoplasm to oxalate by lactate dehydrogenase (LDHA). While glycolate is a harmless intermediate of the glycine metabolism pathway, accumulation of glyoxylate (via, e.g., AGXT mutation) drives oxalate accumulation, which ultimately results in the PH1 disease.

The present invention requires the use of site-specific, rare-cutting nucleases that are engineered to recognize DNA sequences within the HAO1 gene sequence. Methods for producing engineered, site-specific nucleases are known in the art. For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR endonuclease comprises two components: (1) a caspase effector nuclease; and (2) a short “guide RNA” comprising a ˜20 nucleotide targeting sequence that directs the nuclease to a location of interest in the genome. By expressing multiple guide RNAs in the same cell, each having a different targeting sequence, it is possible to target DNA breaks simultaneously to multiple sites in in the genome.

In an embodiment of the invention, the DNA break-inducing agent is an engineered homing endonuclease (also called a “meganuclease”). Homing endonucleases are a group of naturally-occurring nucleases which recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Homing endonucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG homing endonucleases with a single copy of the LAGLIDADG motif form homodimers, whereas members with two copies of the LAGLIDADG motif are found as monomers.

I-CreI (SEQ ID NO: 1) is a member of the LAGLIDADG family of homing endonucleases which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). Methods for rationally-designing mono-LAGLIDADG homing endonucleases were described which are capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li, et al. (2009) Nucleic Acids Res. 37:1650-62; Grizot, et al. (2009) Nucleic Acids Res. 37:5405-19.) Thus, a functional “single-chain” meganuclease can be expressed from a single transcript. This, coupled with the extremely low frequency of off-target cutting observed with engineered meganucleases makes them the preferred endonuclease for the present invention.

The present invention improves upon previous gene editing approaches for targeting the HAO1 gene and treating PHL The HAO1 gene consists of eight exons separated by large intron sequences. In a conventional editing approach, an exon toward the 5′ end of the gene would be targeted in order to disrupt expression of the protein. However, provided herein is an unconventional approach which targets exon 8 of HAO1, the most downstream coding sequence of the gene. Exon 8 is highly conserved across species, with only a one base pair difference between the human, rhesus monkey, and mouse HAO1 genes Importantly, the present approach generates a mutation in exon 8 that disrupts coding of the C-terminal SKI motif. The SKI motif is a non-canonical peroxisomal targeting signal (PTS) that is essential for transport of the HAO1 protein into the peroxisome, where the HAO1 protein catalyzes the conversion of glycolate to glyoxylate. The absence of the SKI motif results in an HAO1 protein that is largely intact and potentially active, but not localized to the peroxisome. As a result, levels of the glycolate substrate in cells expressing the modified HAO1 gene will be elevated, while levels of glyoxylate in the peroxisome, and oxalate in the cytoplasm, will be reduced. This approach is effective because glycolate is a highly soluble small molecule that can be eliminated at high concentrations in the urine without affecting the kidney. The surprising effectiveness of this alternative gene editing approach is demonstrated herein using in vitro models and in vivo studies, as further outlined in the Examples.

Accordingly, the present invention fulfills a need in the art for gene therapy approaches to treat PH1.

### SUMMARY OF THE INVENTION

The present invention provides engineered nucleases that bind and cleave a recognition sequence within or adjacent to exon 8 of an HAO1 gene (SEQ ID NO: 4) such that coding of the HAO1 peroxisomal targeting signal (i.e., SKI motif) is disrupted, thereby limiting peroxisomal localization of the HAO1 gene product. The present invention further provides methods comprising the delivery of an engineered protein, or genes encoding an engineered nuclease, to a eukaryotic cell in order to produce a genetically-modified eukaryotic cell. The present invention also provides pharmaceutical compositions and methods for treatment of primary hyperoxaluria and reduction of serum oxalate levels which utilize an engineered nuclease having specificity for a recognition sequence positioned within or adjacent to exon 8 of a HAO1 gene.

Thus, in one aspect, the invention provides an engineered meganuclease that binds and cleaves a recognition sequence comprising SEQ ID NO: 5 within an HAO1 gene, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region.

In one embodiment, the HVR1 region comprises an amino acid sequence having at least 80%, at least 85%, at least 90%, at least 95%, or more, sequence identity to an amino acid sequence corresponding to residues 24-79 of any one of SEQ ID NOs: 7, 8, 9, or 10.

In some such embodiments, the HVR1 region comprises one or more residues corresponding to residues 24, 26, 28, 30, 32, 33, 38, 40, 42, 44, 46, 68, 70, 75, and 77 of any one of SEQ ID NOs: 7, 8, 9, or 10.

In some such embodiments, the HVR1 region comprises residues corresponding to residues 24, 26, 28, 30, 32, 33, 38, 40, 42, 44, 46, 68, 70, 75, and 77 of any one of SEQ ID NOs: 7, 8, 9, or 10.

In certain embodiments, the HVR1 region comprises Y, R, K, or D at a residue corresponding to residue 66 of any one of SEQ ID NOs: 7, 8, 9, or 10. In particular embodiments, the HVR1 region comprises residues 24-79 of any one of SEQ ID NOs: 7, 8, 9, or 10.

In some such embodiments, the HVR2 region comprises an amino acid sequence having at least 80%, at least 85%, at least 90%, at least 95%, or more, sequence identity to an amino acid sequence corresponding to residues 215-270 of any one of SEQ ID NOs: 7, 8, 9, or 10.

In certain embodiments, the HVR2 region comprises one or more residues corresponding to residues 215, 217, 219, 221, 223, 224, 229, 231, 233, 235, 237, 259, 261, 266, and 268 of any one of SEQ ID NOs: 7, 8, 9, or 10.

In certain embodiments, the HVR2 region comprises residues corresponding to residues 215, 217, 219, 221, 223, 224, 229, 231, 233, 235, 237, 259, 261, 266, and 268 of any one of SEQ ID NOs: 7, 8, 9, or 10.

In certain embodiments, the HVR2 region comprises Y, R, K, or D at a residue corresponding to residue 257 of any one of SEQ ID NOs: 7, 8, 9, or 10.

In certain embodiments, the HVR2 region comprises residues corresponding to residues 239 and 241 of SEQ ID NO: 9.

In certain embodiments, the HVR2 region comprises residues corresponding to residues 239, 241, 262, 263, 264, and 265 of SEQ ID NO: 10.

In certain embodiments, the HVR2 region comprises residues 215-270 of any one of SEQ ID NOs: 7, 8, 9, or 10.

In one such embodiment, the first subunit comprises an amino acid sequence having at least 80%, at least 85%, at least 90%, at least 95%, or more, sequence identity to residues 7-153 of any one of SEQ ID NOs: 7, 8, 9, or 10, and wherein the second subunit comprises an amino acid sequence having at least 80%, at least 85%, at least 90%, at least 95%, or more, sequence identity to residues 198-344 of any one of SEQ ID NOs: 7, 8, 9, or 10.

In certain embodiments, the first subunit comprises G, S, or A at a residue corresponding to residue 19 of any one of SEQ ID NOs: 7, 8, 9, or 10.

In certain embodiments, the first subunit comprises E, Q, or K at a residue corresponding to residue 80 of any one of SEQ ID NOs: 7, 8, 9, or 10. In certain embodiments, the first subunit comprises a residue corresponding to residue 80 of any one of SEQ ID NOs: 7, 8, 9, or 10.

In certain embodiments, the second subunit comprises G, S, or A at a residue corresponding to residue 210 of any one of SEQ ID NOs: 7, 8, 9, or 10.

In certain embodiments, the second subunit comprises E, Q, or K at a residue corresponding to residue 271 of any one of SEQ ID NOs: 7, 8, 9, or 10. In another such embodiment, the second subunit comprises a residue corresponding to residue 271 of any one of SEQ ID NOs: 7, 8, 9, or 10.

In certain embodiments, the second subunit comprises a residue corresponding to residue 330 of any one of SEQ ID NOs: 9 or 10.

In certain embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 7, 8, 9, or 10.

In particular embodiments, the engineered meganuclease comprises the amino acid sequence of any one of SEQ ID NOs: 7, 8, 9, or 10.

In another aspect, the invention provides a polynucleotide comprising a nucleic acid sequence encoding any engineered meganuclease of the invention. In a particular embodiment, the polynucleotide can be an mRNA. In certain embodiments, the polynucleotide is an isolated polynucleotide.

In another aspect, the invention provides a recombinant DNA construct comprising a nucleic acid sequence encoding any engineered meganuclease of the invention.

In one such embodiment, the recombinant DNA construct encodes a viral vector comprising the nucleic acid sequence encoding the engineered meganuclease. In such an embodiment, the viral vector can be an adenoviral vector, a lentiviral vector, a retroviral vector, or an adeno-associated viral (AAV) vector. In a particular embodiment, the viral vector is a recombinant AAV vector.

In another aspect, the invention provides a viral vector comprising a nucleic acid sequence which encodes any engineered meganuclease of the invention. In one embodiment, the viral vector can be an adenoviral vector, a lentiviral vector, a retroviral vector, or an adeno-associated viral (AAV) vector. In a particular embodiment, the viral vector can be a recombinant AAV vector.

In another aspect, the invention provides a method for producing a genetically-modified eukaryotic cell comprising an exogenous sequence of interest inserted into a chromosome of the eukaryotic cell, the method comprising introducing into a eukaryotic cell one or more nucleic acids including: (a) a first nucleic acid encoding any engineered meganuclease of the invention, wherein the engineered meganuclease is expressed in the eukaryotic cell; and (b) a second nucleic acid including the sequence of interest; wherein the engineered meganuclease produces a cleavage site in the chromosome at a recognition sequence comprising SEQ ID NO: 5; and wherein the sequence of interest is inserted into the chromosome at the cleavage site.

In one embodiment of the method, the second nucleic acid further comprises sequences homologous to sequences flanking the cleavage site and the sequence of interest is inserted at the cleavage site by homologous recombination.

In another embodiment of the method, the eukaryotic cell is a mammalian cell. In one such embodiment, the mammalian cell is selected from a human cell, non-human primate cell, or a mouse cell. In one embodiment, the mammalian cell is a hepatocyte. In certain embodiments, the hepatocyte is within the liver of a human, a non-human primate, or a mouse.

In another embodiment of the method, the first nucleic acid is introduced into the eukaryotic cell by an mRNA or a viral vector. In one such embodiment, the mRNA can be packaged within a lipid nanoparticle. In another such an embodiment, the viral vector can be an adenoviral vector, a lentiviral vector, a retroviral vector, or an adeno-associated viral (AAV) vector. In a particular embodiment, the viral vector can be a recombinant AAV vector.

In some embodiments of the method, the second nucleic acid is introduced into the eukaryotic cell by a viral vector. In such an embodiment, the viral vector can be an adenoviral vector, a lentiviral vector, a retroviral vector, or an adeno-associated viral (AAV) vector. In a particular embodiment, the viral vector can be a recombinant AAV vector.

In another aspect, the invention provides a method for producing a genetically-modified eukaryotic cell comprising an exogenous sequence of interest inserted into a chromosome of the eukaryotic cell, the method comprising: (a) introducing any engineered meganuclease of the invention into a eukaryotic cell; and (b) introducing a nucleic acid including the sequence of interest into the eukaryotic cell; wherein the engineered meganuclease produces a cleavage site in the chromosome at a recognition sequence comprising SEQ ID NO: 5; and wherein the sequence of interest is inserted into the chromosome at the cleavage site.

In one embodiment of the method, the nucleic acid further comprises sequences homologous to sequences flanking the cleavage site and the sequence of interest is inserted at the cleavage site by homologous recombination.

In some embodiments of the method, the eukaryotic cell is a mammalian cell. In some embodiments, the mammalian cell is selected from a human cell, non-human primate cell, or a mouse cell. IN particular embodiments, the mammalian cell is a hepatocyte. In some embodiments, the hepatocyte is within the liver of a human, a non-human primate, or a mouse.

In some embodiments of the method, the nucleic acid is introduced into the eukaryotic cell by a viral vector. In such an embodiment, the viral vector can be an adenoviral vector, a lentiviral vector, a retroviral vector, or an adeno-associated viral (AAV) vector. In a particular embodiment, the viral vector can be a recombinant AAV vector.

In another aspect, the invention provides a method for producing a genetically-modified eukaryotic cell by disrupting a target sequence in a chromosome of the eukaryotic cell, the method comprising introducing into a eukaryotic cell a nucleic acid encoding any engineered meganuclease of the invention, wherein the engineered meganuclease is expressed in the eukaryotic cell; wherein the engineered meganuclease produces a cleavage site in the chromosome at a recognition sequence comprising SEQ ID NO: 5, and wherein the target sequence is disrupted by non-homologous end-joining at the cleavage site.

In some embodiments of the method, the disruption produces a modified HAO1 gene which encodes a modified HAO1 polypeptide, wherein the modified HAO1 polypeptide comprises the amino acids encoded by exons 1-7 of the HAO1 gene but lacks a peroxisomal targeting signal.

In some embodiments of the method, the disruption produces a modified HAO1 gene which encodes a modified HAO1 polypeptide having at least 80%, 85%, 90%, 95%, 96%, 97%, 98%, 99%, or 100% sequence identity to the nucleotide sequence of SEQ ID NO: 22.

In some embodiments of the method, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In some embodiments of the method, the nucleic acid is introduced into the eukaryotic cell by an mRNA or a viral vector. In one such embodiment, the mRNA can be packaged within a lipid nanoparticle. In another such embodiment, the viral vector can be an adenoviral vector, a lentiviral vector, a retroviral vector, or an adeno-associated viral (AAV) vector. In a particular embodiment, the viral vector can be a recombinant AAV vector.

In another aspect, the invention provides a method for producing a genetically-modified eukaryotic cell by disrupting a target sequence in a chromosome of the eukaryotic cell, the method comprising introducing into a eukaryotic cell any engineered meganuclease of the invention; wherein the engineered meganuclease produces a cleavage site in the chromosome at a recognition sequence comprising SEQ ID NO: 5, and wherein the target sequence is disrupted by non-homologous end-joining at the cleavage site.

In some embodiments of the method, the disruption produces a modified HAO1 gene which encodes a modified HAO1 polypeptide, wherein the modified HAO1 polypeptide comprises the amino acids encoded by exons 1-7 of the HAO1 gene but lacks a peroxisomal targeting signal.

In some embodiments of the method, the disruption produces a modified HAO1 gene which encodes a modified HAO1 polypeptide having at least 80%, 85%, 90%, 95%, 96%, 97%, 98%, 99%, or 100% sequence identity to the nucleotide sequence of SEQ ID NO: 22.

In some embodiments of the method, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a genetically-modified eukaryotic cell prepared by any method described herein of producing a genetically-modified eukaryotic cell of the invention.

In another aspect, the invention provides a genetically-modified eukaryotic cell comprising a modified HAO1 gene, wherein the modified HAO1 gene encodes a modified HAO1 polypeptide which comprises the amino acids encoded by exons 1-7 of the HAO1 gene but lacks a peroxisomal targeting signal.

In some embodiments of the genetically-modified eukaryotic cell, the modified HAO1 gene encodes a modified HAO1 polypeptide having at least 80%, 85%, 90%, 95%, 96%, 97%, 98%, 99%, or 100% sequence identity to the nucleotide sequence of SEQ ID NO: 22.

In some embodiments of the genetically-modified eukaryotic cell, the modified HAO1 gene comprises a nucleic acid insertion or deletion within exon 8 which disrupts coding of the peroxisomal targeting signal.

In some embodiments of the genetically-modified eukaryotic cell, the insertion or deletion is positioned only within exon 8, spans the junction of exon 8 and the 5′ upstream intron, or spans the junction of exon 8 and the 3′ downstream intron.

In some embodiments of the genetically-modified eukaryotic cell, the modified HAO1 polypeptide is not localized to the peroxisome (e.g., as detected using standard methods in the art, e.g., microscopy, e.g., immunofluorescence microscopy; See Example 5). In some embodiments, localization of the modified HAO1 polypeptide to the peroxisome is reduced by at least 1%, at least 5%, at least 10%, at least about 20%, at least about 30%, at least about 40%, at least about 50%, at least about 60%, at least about 70%, at least about 80%, at least about 90%, or up to 100% relative to a control.

In some embodiments of the genetically-modified eukaryotic cell, the conversion of glycolate to glyoxylate is reduced (e.g., as determined by measurements of glycolate and/or glyoxylate levels) in the genetically-modified eukaryotic cell relative to a control (e.g., a control cell). For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In some embodiments, the conversion of glycolate to glyoxylate is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the conversion of glycolate to glyoxylate is reduced by 1-5%, 5%-10%, 10%-20%, 20%-30%, 30%-40%, 40%-50%, 50%-60%, 70%-80%, 90-95%, 95-98%, or up to 100% relative to the control.

In some embodiments of the genetically-modified eukaryotic cell, the production of oxalate (e.g., as determined by measurements of oxalate levels) is reduced in the genetically-modified eukaryotic cell relative to a control (e.g., a control cell). For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In some embodiments, the production of oxalate is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the control. In some embodiments, the production of oxalate is reduced by 1%-5%, 5%-10%, 10%-20%, 20%-30%, 30%-40%, 40%-50%, 50%-60%, 70%-80%, 90%-95%, 95%-98%, or 100% relative to the control.

In some embodiments of the genetically-modified eukaryotic cell, the insertion or deletion is positioned at an engineered nuclease cleavage site.

In some embodiments of the genetically-modified eukaryotic cell, the engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

In some embodiments of the genetically-modified eukaryotic cell, the engineered nuclease cleavage site is within an engineered meganuclease recognition sequence, a TALEN recognition sequence, a zinc finger nuclease (ZFN) recognition sequence, a CRISPR system nuclease recognition sequence, a compact TALEN recognition sequence, or a megaTAL recognition sequence.

In some embodiments of the genetically-modified eukaryotic cell, the engineered nuclease cleavage site is within an engineered meganuclease recognition sequence comprising any one of SEQ ID NOs: 5, 23, or 24. In some embodiments, the engineered meganuclease recognition sequence comprises SEQ ID NO: 5.

In some embodiments of the genetically-modified eukaryotic cell, the engineered nuclease cleavage site is a TALEN cleavage site within a TALEN spacer sequence comprising any one of SEQ ID NOs: 53-96.

In some embodiments of the genetically-modified eukaryotic cell, the engineered nuclease cleavage site is a zinc finger nuclease cleavage site within a zinc finger nuclease spacer sequence comprising any one of SEQ ID NOs: 25-52.

In some embodiments of the genetically-modified eukaryotic cell, the engineered nuclease cleavage site is within a CRISPR system nuclease recognition sequence comprising any one of SEQ ID NOs: 97-115.

In some embodiments, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a method for producing a genetically-modified eukaryotic cell comprising a modified HAO1 gene, the method comprising introducing into a eukaryotic cell: (a) a nucleic acid encoding an engineered nuclease having specificity for a recognition sequence within an HAO1 gene, wherein the engineered nuclease is expressed in the eukaryotic cell; or (b) the engineered nuclease having specificity for a recognition sequence within an HAO1 gene; wherein the engineered nuclease produces a cleavage site within the recognition sequence and generates a modified HAO1 gene which encodes a modified HAO1 polypeptide, wherein the modified HAO1 polypeptide comprises the amino acids encoded by exons 1-7 of the HAO1 gene but lacks a peroxisomal targeting signal.

In some embodiments of the method, the modified HAO1 gene encodes a modified HAO1 polypeptide having at least 80%, 85%, 90%, 95%, 96%, 97%, 98%, 99%, or 100% sequence identity to the nucleotide sequence of SEQ ID NO: 22.

In some embodiments of the method, the engineered nuclease has specificity for a recognition sequence positioned within or adjacent to exon 8 of the HAO1 gene.

In some embodiments, the recognition sequence positioned adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 5′ upstream of exon 8. In some embodiments, the recognition sequence positioned adjacent to exon 8 is positioned 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the recognition sequence positioned adjacent to exon 8 is positioned within 10 bp 5′ upstream of exon 8.

In some embodiments, the recognition sequence positioned adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 3′ downstream of exon 8. In some embodiments, the recognition sequence positioned adjacent to exon 8 is positioned up to 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the recognition sequence positioned adjacent to exon 8 is positioned within 10 bp 3′ downstream of exon 8.

In some embodiments of the method, the modified HAO1 gene comprises an insertion or deletion within exon 8 which disrupts coding of the peroxisomal targeting signal.

In some embodiments of the method, the insertion or deletion is positioned only within exon 8, spans the junction of exon 8 and the 5′ upstream intron, or spans the junction of exon 8 and the 3′ downstream intron.

In some embodiments of the method, the modified HAO1 polypeptide is not localized to the peroxisome (e.g., as detected using standard methods in the art, e.g., microscopy, e.g., immunofluorescence microscopy; See Example 5). In some embodiments, localization of the modified HAO1 polypeptide to the peroxisome is reduced by at least 1%, at least 5%, at least 10%, at least about 20%, at least about 30%, at least about 40%, at least about 50%, at least about 60%, at least about 70%, at least about 80%, at least about 90%, or up to 100% relative to a control.

In some embodiments of the method, the conversion of glycolate to glyoxylate is reduced (e.g., as determined by measurements of glycolate and/or glyoxylate levels) in the genetically-modified eukaryotic cell relative to a control (e.g., a control cell). For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In some embodiments, the conversion of glycolate to glyoxylate is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to100% relative to the control. In some embodiments, the conversion of glycolate to glyoxylate is reduced by 1-5%, 5%-10%, 10%-20%, 20%-30%, 30%-40%, 40%-50%, 50%-60%, 70%-80%, 90-95%, 95-98%, or up to 100% relative to the control.

In some embodiments of the method, the production of oxalate is reduced (e.g., as determined by measurements of oxalate levels) in the genetically-modified eukaryotic cell relative to a control (e.g., a control cell). For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In some embodiments, the production of oxalate is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the production of oxalate is reduced by 1%-5%, 5%-10%, 10%-20%, 20%-30%, 30%-40%, 40%-50%, 50%-60%, 70%-80%, 90-95%, 95-98%, or up to 100% relative to the control.

In some embodiments of the method, the insertion or deletion is introduced at an engineered nuclease cleavage site.

In some embodiments of the method, the engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

In some embodiments of the method, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 5′ upstream of exon 8. In some embodiments, the engineered nuclease cleavage site adjacent to exon 8 is positioned 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the engineered nuclease cleavage site adjacent to exon 8 is positioned within 10 bp 5′ upstream of exon 8.

In some embodiments of the method, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 3′ downstream of exon 8. In some embodiments, the engineered nuclease cleavage site adjacent to exon 8 is positioned up to 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the engineered nuclease cleavage site adjacent to exon 8 is positioned within 10 bp 3′ downstream of exon 8.

In some embodiments of the method, the engineered nuclease is an engineered meganuclease, a TALEN, a zinc finger nuclease (ZFN), a CRISPR system nuclease, a compact TALEN, or a megaTAL.

In some embodiments of the method, the engineered nuclease is an engineered meganuclease having specificity for a recognition sequence comprising any one of SEQ ID NOs: 5, 23, or 24. In some embodiments, the engineered meganuclease has specificity for a recognition sequence comprising SEQ ID NO: 5. In particular embodiments, the engineered meganuclease is any engineered meganuclease described herein which has specificity for SEQ ID NO: 5.

In some embodiments of the method, the engineered nuclease is a TALEN which generates the cleavage site within a TALEN spacer sequence comprising any one of SEQ ID NOs: 53-96.

In some embodiments of the method, the engineered nuclease is a zinc finger nuclease which generates the cleavage site within a zinc finger nuclease spacer sequence comprising any one of SEQ ID NOs: 25-52.

In some embodiments of the method, the engineered nuclease is a CRISPR system nuclease which generates the cleavage site within a CRISPR system nuclease recognition sequence comprising any one of SEQ ID NOs: 97-115.

In some embodiments of the method, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In some embodiments, the nucleic acid is introduced into the eukaryotic cell by an mRNA or a viral vector. In one such embodiment, the mRNA can be packaged within a lipid nanoparticle. In another such an embodiment, the viral vector can be an adenoviral vector, a lentiviral vector, a retroviral vector, or an adeno-associated viral (AAV) vector. In a particular embodiment, the viral vector can be a recombinant AAV vector.

In another aspect, the invention provides a pharmaceutical composition comprising a pharmaceutically-acceptable carrier and any engineered nuclease provided herein, or a nucleic acid encoding any such engineered nuclease.

In another aspect, the invention provides a pharmaceutical composition comprising a pharmaceutically acceptable carrier and: (a) a nucleic acid encoding an engineered nuclease having specificity for a recognition sequence within an HAO1 gene, wherein the engineered nuclease is expressed in a eukaryotic cell in vivo; or (b) an engineered nuclease having specificity for a recognition sequence within an HAO1 gene; wherein the engineered nuclease produces a cleavage site within the recognition sequence and generates a modified HAO1 polypeptide, wherein the modified HAO1 polypeptide comprises the amino acids encoded by exons 1-7 of the HAO1 gene but lacks a peroxisomal targeting signal.

In some embodiments of the pharmaceutical composition, the modified HAO1 gene encodes a modified HAO1 polypeptide having at least 80%, 85% 90%, 95%, 96%, 97%, 98%, 99%, or 100% sequence identity to the nucleotide sequence of SEQ ID NO: 22.

In some embodiments of the pharmaceutical composition, the modified HAO1 gene comprises an insertion or deletion within exon 8 which disrupts coding of the peroxisomal targeting signal.

In some embodiments of the pharmaceutical composition, the insertion or deletion is positioned only within exon 8, spans the junction of exon 8 and the 5′ upstream intron, or spans the junction of exon 8 and the 3′ downstream intron.

In some embodiments of the pharmaceutical composition, the modified HAO1 polypeptide does not localize to the peroxisome (e.g., as detected using standard methods in the art, e.g., microscopy, e.g., immunofluorescence microscopy; See Example 5). In some embodiments, localization of the modified HAO1 polypeptide to the peroxisome is reduced by at least 1%, at least 5%, at least 10%, at least about 20%, at least about 30%, at least about 40%, at least about 50%, at least about 60%, at least about 70%, at least about 80%, at least about 90%, or up to 100% relative to a control.

In some embodiments of the pharmaceutical composition, the insertion or deletion is positioned at the engineered nuclease cleavage site.

In some embodiments of the pharmaceutical composition, the engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

In some embodiments of the pharmaceutical composition, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 5′ upstream of exon 8. In some embodiments, the engineered nuclease cleavage site adjacent to exon 8 is positioned 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the engineered nuclease cleavage site adjacent to exon 8 is positioned within 10 bp 5′ upstream of exon 8.

In some embodiments of the pharmaceutical composition, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 3′ downstream of exon 8. In some embodiments, the engineered nuclease cleavage site adjacent to exon 8 is positioned up to 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the engineered nuclease cleavage site adjacent to exon 8 is positioned within 10 bp 3′ downstream of exon 8.

In some embodiments of the pharmaceutical composition, the engineered nuclease is an engineered meganuclease, a TALEN, a zinc finger nuclease (ZFN), a CRISPR system nuclease, a compact TALEN, or a megaTAL.

In some embodiments of the pharmaceutical composition, the engineered nuclease is an engineered meganuclease having specificity for a recognition sequence of any one of SEQ ID NOs: 5, 23, or 24.

In some embodiments of the pharmaceutical composition, the engineered meganuclease recognition sequence comprises SEQ ID NO: 5. In particular embodiments, the engineered meganuclease is any engineered meganuclease described herein which has specificity for SEQ ID NO: 5.

In some embodiments of the pharmaceutical composition, the engineered nuclease is a TALEN which generates the cleavage site within a TALEN spacer sequence comprising any one of SEQ ID NOs: 53-96.

In some embodiments of the pharmaceutical composition, the engineered nuclease is a zinc finger nuclease which generates the cleavage site within a zinc finger nuclease spacer sequence comprising any one of SEQ ID NOs: 25-52.

In some embodiments of the pharmaceutical composition, the engineered nuclease is a CRISPR system nuclease having specificity for a recognition sequence of any one of SEQ ID NOs: 97-115.

In some embodiments of the pharmaceutical composition, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In some embodiments of the pharmaceutical composition, the nucleic acid is an mRNA. In some embodiments, the mRNA is encapsulated in a lipid nanoparticle.

In some embodiments of the pharmaceutical composition, the pharmaceutical composition comprises a recombinant DNA construct comprising the nucleic acid.

In some embodiments of the pharmaceutical composition, the pharmaceutical composition comprises a viral vector comprising the nucleic acid. In some embodiments the viral vector is a recombinant AAV vector.

In some embodiments of the pharmaceutical composition, the pharmaceutical composition is for the treatment of a subject having primary hyperoxaluria.

In another aspect, the invention provides a method for reducing serum oxalate levels in vivo, the method comprising delivering to a target cell any engineered meganuclease of the invention, or a nucleic acid encoding any engineered meganuclease of the invention, wherein the method is effective to reduce the conversion of glycolate to glyoxylate (e.g., as determined by measurements of glycolate and/or glyoxylate levels) in vivo relative to a reference level.

In another aspect, the invention provides a method for reducing serum oxalate levels in vivo, the method comprising delivering to a target cell: (a) a nucleic acid encoding an engineered nuclease having specificity for a recognition sequence within an HAO1 gene, wherein the engineered nuclease is expressed in the target cell; or (b) the engineered nuclease having specificity for a recognition sequence within an HAO1 gene; wherein the engineered nuclease produces a cleavage site within the recognition sequence and generates a modified HAO1 gene which encodes a modified HAO1 polypeptide, wherein the modified HAO1 polypeptide comprises the amino acids encoded by exons 1-7 of the HAO1 gene but lacks a peroxisomal targeting signal, and wherein the method is effective to reduce the conversion of glycolate to glyoxylate (e.g., as determined by measurements of glycolate and/or glyoxylate levels) in vivo relative to a reference level.

In some embodiments of the method, the modified HAO1 gene encodes a modified HAO1 polypeptide having at least 80%, 85%, 90%, 95%, 96%, 97%, 98%, 99%, or 100% sequence identity to the nucleotide sequence of SEQ ID NO: 22.

In some embodiments of the method, the engineered nuclease has specificity for a recognition sequence positioned within or adjacent to exon 8 of the HAO1 gene.

In some embodiments, the recognition sequence positioned adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 5′ upstream of exon 8. In some embodiments, the recognition sequence positioned adjacent to exon 8 is positioned 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the recognition sequence positioned adjacent to exon 8 is positioned within 10 bp 5′ upstream of exon 8.

In some embodiments, the recognition sequence positioned adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 3′ downstream of exon 8. In some embodiments, the recognition sequence positioned adjacent to exon 8 is positioned up to 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the recognition sequence positioned adjacent to exon 8 is positioned within 10 bp 3′ downstream of exon 8.

In some embodiments of the method, the modified HAO1 gene comprises an insertion or deletion within exon 8 which disrupts coding of the peroxisomal targeting signal.

In some embodiments of the method, the insertion or deletion is positioned only within exon 8, spans the junction of exon 8 and the 5′ upstream intron, or spans the junction of exon 8 and the 3′ downstream intron.

In some embodiments of the method, the modified HAO1 polypeptide is not localized to the peroxisome (e.g., as detected using standard methods in the art, e.g., microscopy, e.g., immunofluorescence microscopy; See Example 5). In some embodiments, localization of the modified HAO1 polypeptide to the peroxisome is reduced by at least 1%, at least 5%, at least 10%, at least about 20%, at least about 30%, at least about 40%, at least about 50%, at least about 60%, at least about 70%, at least about 80%, at least about 90%, or up to 100% relative to a control.

In some embodiments of the method, the insertion or deletion is introduced at the engineered nuclease cleavage site.

In some embodiments of the method, the engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

In some embodiments, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 5′ upstream of exon 8. In some embodiments, the engineered nuclease cleavage site adjacent to exon 8 is positioned 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the engineered nuclease cleavage site adjacent to exon 8 is positioned within 10 bp 5′ upstream of exon 8.

In some embodiments, the engineered nuclease cleavage site adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, or 1 bp 3′ downstream of exon 8. In some embodiments, the engineered nuclease cleavage site adjacent to exon 8 is positioned up to 1 bp, 2 bp, 1-3 bp, 1-4 bp, 1-5 bp, 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the engineered nuclease cleavage site adjacent to exon 8 is positioned within 10 bp 3′ downstream of exon 8.

In some embodiments of the method, the engineered nuclease is an engineered meganuclease, a TALEN, a zinc finger nuclease (ZFN), a CRISPR system nuclease, a compact TALEN, or a megaTAL.

In some embodiments of the method, the engineered nuclease is an engineered meganuclease having specificity for a recognition sequence comprising any one of SEQ ID NOs: 5, 23, or 24. In some embodiments, the engineered meganuclease has specificity for a recognition sequence comprising SEQ ID NO: 5. In particular embodiments, the engineered meganuclease is any engineered meganuclease described herein which has specificity for SEQ ID NO: 5.

In some embodiments of the method, the engineered nuclease is a TALEN which generates the cleavage site within a TALEN spacer sequence comprising any one of SEQ ID NOs: 53-96.

In some embodiments of the method, the engineered nuclease is a zinc finger nuclease which generates the cleavage site within a zinc finger nuclease spacer sequence comprising any one of SEQ ID NOs: 25-52.

In some embodiments of the method, the engineered nuclease is a CRISPR system nuclease having specificity for a recognition sequence comprising any one of SEQ ID NOs: 97-115.

In some embodiments of the method, the method is effective to reduce the level of serum oxalate in vivo relative to a reference level.

In some embodiments of the method, the target cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a method for treating primary hyperoxyluria-1 (PH1) in a subject in need thereof, wherein the method comprises administering to the subject an effective amount of any pharmaceutical composition of the invention.

In some embodiments, the method is effective to reduce serum oxalate levels in the subject relative to a reference level. In some embodiments of the method, the reference level is the level of serum oxalate in a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the serum oxalate level is reduced in the subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the reference level. In some embodiments, the serum oxalate level is reduced in the subject by 1%-5%, 5%-10%, 10%-20%, 20%-30%, 30%-40%, 40%-50%, 50%-60%, 70%-80%, 90%-95%, 95%-98%, or up to 100% relative to the reference level. In some embodiments, the method is effective to reduce serum oxalate levels in the subject to undetectable levels, or to less than 1% 2%, 5%, 10%, 20%, 30%, 40%, 50%, 60%, 70%, or 80% of the subject's oxalate levels prior to treatment (e.g., within 1, 3, 5, 7, 9, 12, or 15 days).

In some embodiments, the method is effective to reduce urinary oxalate levels in the subject relative to a reference level. In some embodiments of the method, the reference level is the level of urinary oxalate in a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the urinary oxalate level is reduced in the subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the reference level. In some embodiments, the urinary oxalate level is reduced in the subject by 1%-5%, 5%-10%, 10%-20%, 20%-30%, 30%-40%, 40%-50%, 50%-60%, 70%-80%, 90%-95%, 95-98%, or up to 100% relative to the reference level. In some embodiments, the method is effective to reduce urinary oxalate levels in the subject to undetectable levels, or to less than 1% 2%, 5%, 10%, 20%, 30%, 40%, 50%, 60%, 70%, or 80% of the subject's oxalate levels prior to treatment (e.g., within 1, 3, 5, 7, 9, 12, or 15 days).

In some embodiments, the method is effective to increase a glycolate/creatinine ratio in a urine sample from the subject and decrease an oxalate/creatinine ratio in a urine sample from the subject relative to a reference level. In some embodiments of the method, the reference level is the oxalate/creatinine ratio and/or glycolate/creatinine ratio in a urine sample in a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the oxalate/creatinine ratio is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the reference level. In some embodiments, the oxalate/creatinine ratio is reduced by 1%-5%, 5%-10%, 10%-20%, 20%-30%, 30%-40%, 40%-50%, 50%-60%, 70%-80%, 90%-95%, 95%-98%, or up to 100% relative to the reference level.

In some embodiments, the glycolate/creatinine ratio is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 100%, or more, relative to the reference level. In some embodiments, the glycolate/creatinine ratio is increased by at least about 2λ-fold, at least about 3λ-fold, at least about 4λ-fold, at least about 5λ-fold, at least about 6λ-fold, at least about 7λ-fold, at least about 8λ-fold, at least about 9λ-fold, or at least about 10λ-fold relative to the reference level.

In some embodiments, the method is effective to decrease the level of calcium precipitates in a kidney of the subject relative to a reference level. In some embodiments, the reference level is the level of calcium precipitates in the kidney of a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the level of calcium precipitates is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the reference level. In some embodiments, the level of calcium precipitates is reduced by 1%-5%, 5%-10%, 10%-20%, 20%-30%, 30%-40%, 40%-50%, 50%-60%, 70%-80%, 90%-95%, 95%-98%, or 100% relative to the reference level.

In some embodiments, the method is effective to decrease the risk of renal failure in the subject relative to a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the risk of renal failure is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the reference level. In some embodiments, the risk of renal failure is reduced by 1%-5%, 5%-10%, 10%-20%, 20%-30%, 30%-40%, 40%-50%, 50%-60%, 70%-80%, 90%-95%, 95%-98%, or 100% relative to the reference level.

In some embodiments, the subject is a human subject.

In some embodiments, the subject has a mutation in the gene encoding alanine glyoxylate aminotransferase (AGT) that results in accumulation of oxalate.

In some embodiments, the subject is one having urinary oxalate levels of at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mg of oxalate per 24 hour period.

In another aspect, the invention provides a recombinant HAO1 polypeptide comprising the amino acids encoded by exons 1-7 of the HAO1 gene but lacking a functional peroxisomal targeting signal. In some embodiments, the polypeptide is encoded by exons 1-7 and at least 3 bp of exon 8 (SEQ ID NO: 4) but lacks a functional peroxisomal targeting signal (i.e., a SKI motif). In some embodiments, the polypeptide is encoded by exons 1-7 and 3 bp-62 bp (e.g., 3 bp-9 bp, 9 bp-15 bp, 15 bp-21 bp, 21 bp-27 bp, 27 bp-33 bp, 33 bp-39 bp, 39 bp-45 bp, 45 bp-51 bp, 51 bp-57 bp, or 57 bp-62 bp) of exon 8 (SEQ ID NO: 4) but lacks a functional peroxisomal targeting signal (i.e., a SKI motif).

In another aspect, the present disclosure provides an engineered nuclease or a nucleic acid molecule encoding an engineered nuclease, such as an engineered meganuclease, TALEN nuclease, zinc finger nuclease, CRISPR system nuclease, compact TALEN, and/or megaTAL described herein for use as a medicament. The present disclosure further provides the use of an engineered nuclease or a nucleic acid molecule encoding an engineered nuclease described herein in the manufacture of a medicament for treating a disease in a subject in need thereof. In one such embodiment, the medicament is useful in the treatment of PHL In some embodiments, the engineered nuclease or a nucleic acid molecule encoding an engineered nuclease described herein is useful for manufacturing a medicament for reducing serum oxalate levels, reducing urinary oxalate levels, increasing the glycolate/creatinine ratio, decreasing the oxalate/creatinine ratio decreasing the level of calcium precipitates in a kidney of the subject, and/or decreasing the risk of renal failure in a subject, such as a subject with PH1, or a subject with increased serum oxalate levels.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. HAO 1-2 recognition sequence in the human HAO1 gene. A) The HAO 1-2 recognition sequence targeted by engineered meganucleases of the invention comprises two recognition half-sites. Each recognition half-site comprises 9 base pairs, separated by a 4 base pair central sequence. The HAO 1-2 recognition sequence (SEQ ID NO: 5) spans nucleotides 56,810 to 56,831 of the human HAO1 gene (SEQ ID NO: 3), and comprises two recognition half-sites referred to as HAO1 and HAO2.

FIG. 2. The engineered meganucleases of the invention comprise two subunits, wherein the first subunit comprising the HVR1 region binds to a first recognition half-site (e.g., HAO1) and the second subunit comprising the HVR2 region binds to a second recognition half-site (e.g., HAO2). In embodiments where the engineered meganuclease is a single-chain meganuclease, the first subunit comprising the HVR1 region can be positioned as either the N-terminal or C-terminal subunit. Likewise, the second subunit comprising the HVR2 region can be positioned as either the N-terminal or C-terminal subunit.

FIG. 3. Schematic of reporter assay in CHO cells for evaluating engineered meganucleases targeting recognition sequences found in the HAO1 gene (SEQ ID NO: 3). For the engineered meganucleases described herein, a CHO cell line was produced in which a reporter cassette was integrated stably into the genome of the cell. The reporter cassette comprised, in 5′ to 3′ order: an SV40 Early Promoter; the 5′ 2/3 of the GFP gene; the recognition sequence for an engineered meganuclease of the invention (e.g., the HAO 1-2 recognition sequence); the recognition sequence for the CHO-23/24 meganuclease (WO/2012/167192); and the 3′ 2/3 of the GFP gene. Cells stably transfected with this cassette did not express GFP in the absence of a DNA break-inducing agent. Meganucleases were introduced by transduction of plasmid DNA or mRNA encoding each meganuclease. When a DNA break was induced at either of the meganuclease recognition sequences, the duplicated regions of the GFP gene recombined with one another to produce a functional GFP gene. The percentage of GFP-expressing cells could then be determined by flow cytometry as an indirect measure of the frequency of genome cleavage by the meganucleases.

FIG. 4. Efficiency of engineered meganucleases for recognizing and cleaving recognition sequences in the human HAO1 gene (SEQ ID NO: 3) in a CHO cell reporter assay. Each of the engineered meganucleases set forth in SEQ ID NOs: 7 and 8 were engineered to target the HAO 1-2 recognition sequence (SEQ ID NO: 5), and were screened for efficacy in the CHO cell reporter assay. The results shown provide the percentage of GFP-expressing cells observed in each assay, which indicates the efficacy of each meganuclease for cleaving a HAO target recognition sequence or the CHO-23/24 recognition sequence. A negative control (HAO 1-2 bs) was further included in each assay.

FIGS. 5A and 5B. Time course of engineered meganuclease efficacy in CHO cell reporter assay. The HAO 1-2L.5 (SEQ ID NO: 8), HAO 1-2L.30 (SEQ ID NO: 7), HAO 1-2L.285 (SEQ ID NO: 9), and HAO 1-2L.338 (SEQ ID NO: 10) meganucleases were evaluated in the CHO reporter assay, with the percentage of GFP-expressing cells determined 2, 5, and 7 days after introduction of meganuclease-encoding mRNA into the CHO reporter cells. A CHO 23/24 meganuclease was also included at each time point as a positive control. A) Results of CHO cell reporter assay with the HAO 1-2L.5 (SEQ ID NO: 8) and HAO 1-2L.30 (SEQ ID NO: 7) meganucleases along with positive and negative controls. B) Results of CHO cell reporter assay with the HAO 1-2L.30 (SEQ ID NO: 7), HAO 1-2L.285 (SEQ ID NO: 9), and HAO 1-2L.338 (SEQ ID NO: 10) meganucleases along with positive control.

FIGS. 6A and 6B. HAO 1-2 nuclease indels detected using digital PCR. The editing efficiencies of the indicated meganucleases were evaluated at the indicated time points using an indel detection assay. The indicated meganucleases were evaluated against the HAO 1-2 recognition sequence in both HepG2 cells and FL-83b cells using droplet digital PCR. A) Detection of indels in HepG2 cells. B) Detection of indels in FL-83b cells.

FIGS. 7A-7C. HAO 1-2 nuclease indels using digital PCR. The editing efficiencies of the indicated meganucleases were evaluated at the indicated time points using an indel detection assay. The indicated meganucleases were evaluated against the HAO 1-2 target site in both HepG2 cells and FL-83b cells using droplet digital PCR. A) Detection of indels in HepG2 cells. B) Detection of indels in FL-83b cells. C). Detection of indels in FL-83b cells comparing the indel % generated with the HAO 1-2L.30 and HAO 1-2L.30S19 meganucleases.

FIGS. 8A and 8B. Quantitation of glycolate levels in mouse serum of mice administered the HAO 1-2L.30 meganuclease. A) The average pre-bleed level of glycolate in all mice in the treated cohort was 725 ng/ml compared to 83,942 ng/ml in treated mice. Glycolate levels increased 115-fold after injection with AAV encoding the HAO 1-2L.30 meganuclease. B) Elevated levels of glycolate was measured in serum starting at week 1 post injection (>50,000 ng/ml) and continued thru week 8 (>100,000 ng/ml) compared to control mice where no difference was detected in glycolate levels.

FIGS. 9A-9C. Quantitation of indels in mouse liver in mice treated with the HAO 1-2L.30 meganuclease (SEQ ID NO: 7). A) gDNA isolated from mouse livers was used as template in a digital droplet PCR drop off assay. A mouse reference probe was used to calculate percentage of edited HAO1.B) The ratio of deletions to insertions was calculated by deep sequencing. Values were plotted and the slope of the line indicates that this ratio is constant across groups/weeks indicating that editing is not being selected out over time. C) Deep sequence data was analyzed to determine the frequency of deletion, characterizing the most frequent size of deletions generated in HAO 1-2L.30 treated mice.

FIG. 10A-10C. Immunofluorescence of mouse liver treated with HAO 1-2L.30 nuclease. A) A 63× image showing untreated control mouse liver probed with Alexa-647 secondary antibody (red), DAPI (blue), actin cytoskeleton (green). B) A 63× image showing untreated control mouse liver probed with Abcam anti-mouse HAO1 antibody (red), DAPI (blue), actin cytoskeleton (green). C) A 63× image showing HAO 1-2L.30-treated mouse liver probed with Abcam anti-mouse HAO1 antibody (red), DAPI (blue), actin cytoskeleton (green).

FIG. 11. Bar graph showing the percentage of on-target insertions and deletions (indel %) in the endogenous mouse HAO1 gene in AGXT deficient mice by next generation sequencing analysis. AAV containing the HAO 1-2L.30 meganuclease targeting the 1-2 recognition sequence was introduced in the mice at three concentrations (3e11, 3e12, and 3e13 GC/kg). Each bar in the graph represents the indel % for an individual mouse in the study.

FIG. 12A-12C. Graph showing the percent of oxalic acid or glycolate in the urine (FIGS. 12A and 12B) or glycolate in the serum (FIG. 12C) of AGXT deficient mice administered either PBS or an AAV containing the HAO 1-2L.30 meganuclease according to Example 6. The data is normalized to values obtained at day 0 of the study and is shown as a percentage of this baseline value.

FIGS. 13A and 13B. Bar graph showing the percentage of on-target insertions and deletions in an exogenously expressed human HAO1 gene (FIG. 13A) and the endogenous mouse HAO1 gene (FIG. 13B) in Rag-1 deficient mice by next generation sequencing analysis. AAV containing the human HAO1 gene was introduced into the mice at Day 0. At day 14, AAVs containing the HAO 1-2L.30 meganuclease targeting the HAO 1-2 recognition sequence were introduced in the mice at three concentrations (3e10, 3e11, and 3e12 GC/kg). Each bar in the graph represents the indel % for an individual mouse in the study. Both insertion (gray) and deletion rates (black) are indicated on the graphs for mouse and human HAO1 target sites.

FIGS. 14A and 14B. Graph showing the percent of glycolate in the urine (FIG. 14A) and serum (FIG. 14B) of Rag-1 deficient mice administered either PBS or an AAV containing the HAO 1-2L.30 meganuclease or an AAV containing the human HAO1 gene or both according to Example 7. The data is normalized to values obtained at day 0 of the study and is shown as a percentage of this baseline value.

FIG. 15. Bar graph showing the percentage of on-target insertions, deletions, and AAV-inverted terminal repeat (ITR) in the endogenous non-human primate (NHP) HAO 1-2 recognition sequence by next generation sequencing analysis. AAVs containing the HAO 1-2L.30 meganuclease targeting the HAO 1-2 recognition sequence were introduced in Rhesus monkeys at two concentrations (6e12 and 3e13 GC/kg). Each bar in the graph represents the indel % for an individual Rhesus monkey in the study. Insertion (dark gray), deletion rates (light gray), and AAV-ITR integrations (black) are indicated on the graphs for the NHP HAO 1-2 target sites.

### BRIEF DESCRIPTION OF THE SEQUENCES

SEQ ID NO: 1 sets forth the amino acid sequence of the wild-type I-CreI meganuclease from Chlamydomonas reinhardtii.

SEQ ID NO: 2 sets forth the amino acid sequence of the LAGLIDADG motif.

SEQ ID NO: 3 sets forth the nucleic acid sequence of the human HAO1 gene sequence (NCBI GENE ID: 54363).

SEQ ID NO: 4 sets forth the nucleic acid sequence of exon 8 of the human HAO1 gene. SEQ ID NO: 5 sets forth the nucleic acid sequence of the HAO 1-2 recognition sequence (sense strand).

SEQ ID NO: 6 sets forth the nucleic acid sequence of the HAO 1-2 recognition sequence (antisense strand).

SEQ ID NO: 7 sets forth the amino acid sequence of the HAO 1-2L.30 meganuclease. SEQ ID NO: 8 sets forth the amino acid sequence of the HAO 1-2L.5 meganuclease. SEQ ID NO: 9 sets forth the amino acid sequence of the HAO 1-2L.285 meganuclease. SEQ ID NO: 10 sets forth the amino acid sequence of the HAO 1-2L.338 meganuclease. SEQ ID NO: 11 sets forth the amino acid sequence of the HAO 1-2L.30 meganuclease HAO1 halfsite-binding subunit.

SEQ ID NO: 12 sets forth the amino acid sequence of the HAO 1-2L.5 meganuclease HAO1 halfsite-binding subunit.

SEQ ID NO: 13 sets forth the amino acid sequence of the HAO 1-2L.285 meganuclease HAO1 halfsite-binding subunit.

SEQ ID NO: 14 sets forth the amino acid sequence of the HAO 1-2L.338 meganuclease HAO1 halfsite-binding subunit.

SEQ ID NO: 15 sets forth the amino acid sequence of the HAO 1-2L.30 meganuclease HAO2 halfsite-binding subunit.

SEQ ID NO: 16 sets forth the amino acid sequence of the HAO 1-2L.5 meganuclease HAO2 halfsite-binding subunit.

SEQ ID NO: 17 sets forth the amino acid sequence of the HAO 1-2L.285 meganuclease HAO2 halfsite-binding subunit.

SEQ ID NO: 18 sets forth the amino acid sequence of the HAO 1-2L.338 meganuclease HAO2 halfsite-binding subunit.

SEQ ID NO: 19 sets forth the amino acid sequence encoded by exons 1-7 of the human HAO1 gene.

SEQ ID NO: 20 sets forth the amino acids encoded by exons 1-7 of the Macaca mulatta HAO1 gene.

SEQ ID NO: 21 sets forth the amino acids encoded by exons 1-7 of the Mus musculus HAO1 gene.

SEQ ID NO: 22 sets forth the amino acids of a human HAO1 polypeptide lacking a peroxisomal targeting signal (i.e., a SKI domain).

SEQ ID NO: 23 sets forth the nucleic acid sequence of a human HAO1 gene meganuclease recognition sequence (sense strand).

SEQ ID NO: 24 sets forth the nucleic acid sequence of a human HAO1 gene meganuclease recognition sequence (sense strand).

SEQ ID NO: 25 sets forth the nucleic acid sequence of a human HAO1 gene zinc finger nuclease recognition sequence spacer (sense strand).

SEQ ID NO: 26 sets forth the nucleic acid sequence of a human HAO1 gene zinc finger nuclease recognition sequence spacer (sense strand).

SEQ ID NO: 27 sets forth the nucleic acid sequence of a human HAO1 gene zinc finger nuclease recognition sequence spacer (sense strand).

SEQ ID NO: 28 sets forth the nucleic acid sequence of a human HAO1 gene zinc finger nuclease recognition sequence spacer (sense strand).

SEQ ID NO: 29 sets forth the nucleic acid sequence of a human HAO1 gene zinc finger nuclease recognition sequence spacer (sense strand).

SEQ ID NO: 30 sets forth the nucleic acid sequence of a human HAO1 gene zinc finger nuclease recognition sequence spacer (sense strand).

SEQ ID NO: 31 sets forth the nucleic acid sequence of a human HAO1 gene zinc finger nuclease recognition sequence spacer (sense strand).

SEQ ID NO: 32 sets forth the nucleic acid sequence of a human HAO1 gene zinc finger nuclease recognition sequence spacer (sense strand).

SEQ ID NO: 33 sets forth the nucleic acid sequence of a human HAO1 gene zinc finger nuclease recognition sequence spacer (sense strand).

SEQ ID NO: 34 sets forth the nucleic acid sequence of a human HAO1 gene zinc finger nuclease recognition sequence spacer (sense strand).

SEQ ID NO: 35 sets forth the nucleic acid sequence of a human HAO1 gene zinc finger nuclease recognition sequence spacer (sense strand).

SEQ ID NO: 36 sets forth the nucleic acid sequence of a human HAO1 gene zinc finger nuclease recognition sequence spacer (sense strand).

SEQ ID NO: 37 sets forth the nucleic acid sequence of a human HAO1 gene zinc finger nuclease recognition sequence spacer (sense strand).

SEQ ID NO: 38 sets forth the nucleic acid sequence of a human HAO1 gene zinc finger nuclease recognition sequence spacer (sense strand).

SEQ ID NO: 39 sets forth the nucleic acid sequence of a human HAO1 gene zinc finger nuclease recognition sequence spacer (antisense strand).

SEQ ID NO: 40 sets forth the nucleic acid sequence of a human HAO1 gene zinc finger nuclease recognition sequence spacer (antisense strand).

SEQ ID NO: 41 sets forth the nucleic acid sequence of a human HAO1 gene zinc finger nuclease recognition sequence spacer (antisense strand).

SEQ ID NO: 42 sets forth the nucleic acid sequence of a human HAO1 gene zinc finger nuclease recognition sequence spacer (antisense strand).

SEQ ID NO: 43 sets forth the nucleic acid sequence of a human HAO1 gene zinc finger nuclease recognition sequence spacer (antisense strand).

SEQ ID NO: 44 sets forth the nucleic acid sequence of a human HAO1 gene zinc finger nuclease recognition sequence spacer (antisense strand).

SEQ ID NO: 45 sets forth the nucleic acid sequence of a human HAO1 gene zinc finger nuclease recognition sequence spacer (antisense strand).

SEQ ID NO: 46 sets forth the nucleic acid sequence of a human HAO1 gene zinc finger nuclease recognition sequence spacer (antisense strand).

SEQ ID NO: 47 sets forth the nucleic acid sequence of a human HAO1 gene zinc finger nuclease recognition sequence spacer (antisense strand).

SEQ ID NO: 48 sets forth the nucleic acid sequence of a human HAO1 gene zinc finger nuclease recognition sequence spacer (antisense strand).

SEQ ID NO: 49 sets forth the nucleic acid sequence of a human HAO1 gene zinc finger nuclease recognition sequence spacer (antisense strand).

SEQ ID NO: 50 sets forth the nucleic acid sequence of a human HAO1 gene zinc finger nuclease recognition sequence spacer (antisense strand).

SEQ ID NO: 51 sets forth the nucleic acid sequence of a human HAO1 gene zinc finger nuclease recognition sequence spacer (antisense strand).

SEQ ID NO: 52 sets forth the nucleic acid sequence of a human HAO1 gene zinc finger nuclease recognition sequence spacer (antisense strand).

SEQ ID NO: 53 sets forth the nucleic acid sequence of a human HAO 1 gene TALEN nuclease recognition sequence spacer (sense strand).

SEQ ID NO: 54 sets forth the nucleic acid sequence of a human HAO 1 gene TALEN nuclease recognition sequence spacer (sense strand).

SEQ ID NO: 55 sets forth the nucleic acid sequence of a human HAO 1 gene TALEN nuclease recognition sequence spacer (sense strand).

SEQ ID NO: 56 sets forth the nucleic acid sequence of a human HAO 1 gene TALEN nuclease recognition sequence spacer (sense strand).

SEQ ID NO: 57 sets forth the nucleic acid sequence of a human HAO 1 gene TALEN nuclease recognition sequence spacer (sense strand).

SEQ ID NO: 58 sets forth the nucleic acid sequence of a human HAO 1 gene TALEN nuclease recognition sequence spacer (sense strand).

SEQ ID NO: 59 sets forth the nucleic acid sequence of a human HAO 1 gene TALEN nuclease recognition sequence spacer (sense strand).

SEQ ID NO: 60 sets forth the nucleic acid sequence of a human HAO 1 gene TALEN nuclease recognition sequence spacer (sense strand).

SEQ ID NO: 61 sets forth the nucleic acid sequence of a human HAO 1 gene TALEN nuclease recognition sequence spacer (sense strand).

SEQ ID NO: 62 sets forth the nucleic acid sequence of a human HAO 1 gene TALEN nuclease recognition sequence spacer (sense strand).

SEQ ID NO: 63 sets forth the nucleic acid sequence of a human HAO 1 gene TALEN nuclease recognition sequence spacer (sense strand).

SEQ ID NO: 64 sets forth the nucleic acid sequence of a human HAO 1 gene TALEN nuclease recognition sequence spacer (sense strand).

SEQ ID NO: 65 sets forth the nucleic acid sequence of a human HAO 1 gene TALEN nuclease recognition sequence spacer (sense strand).

SEQ ID NO: 66 sets forth the nucleic acid sequence of a human HAO 1 gene TALEN nuclease recognition sequence spacer (sense strand).

SEQ ID NO: 67 sets forth the nucleic acid sequence of a human HAO 1 gene TALEN nuclease recognition sequence spacer (sense strand).

SEQ ID NO: 68 sets forth the nucleic acid sequence of a human HAO 1 gene TALEN nuclease recognition sequence spacer (sense strand).

SEQ ID NO: 69 sets forth the nucleic acid sequence of a human HAO 1 gene TALEN nuclease recognition sequence spacer (sense strand).

SEQ ID NO: 70 sets forth the nucleic acid sequence of a human HAO 1 gene TALEN nuclease recognition sequence spacer (sense strand).

SEQ ID NO: 71 sets forth the nucleic acid sequence of a human HAO 1 gene TALEN nuclease recognition sequence spacer (sense strand).

SEQ ID NO: 72 sets forth the nucleic acid sequence of a human HAO 1 gene TALEN nuclease recognition sequence spacer (antisense strand).

SEQ ID NO: 73 sets forth the nucleic acid sequence of a human HAO 1 gene TALEN nuclease recognition sequence spacer (antisense strand).

SEQ ID NO: 74 sets forth the nucleic acid sequence of a human HAO 1 gene TALEN nuclease recognition sequence spacer (antisense strand).

SEQ ID NO: 75 sets forth the nucleic acid sequence of a human HAO 1 gene TALEN nuclease recognition sequence spacer (antisense strand).

SEQ ID NO: 76 sets forth the nucleic acid sequence of a human HAO 1 gene TALEN nuclease recognition sequence spacer (antisense strand).

SEQ ID NO: 77 sets forth the nucleic acid sequence of a human HAO 1 gene TALEN nuclease recognition sequence spacer (antisense strand).

SEQ ID NO: 78 sets forth the nucleic acid sequence of a human HAO 1 gene TALEN nuclease recognition sequence spacer (antisense strand).

SEQ ID NO: 79 sets forth the nucleic acid sequence of a human HAO 1 gene TALEN nuclease recognition sequence spacer (antisense strand).

SEQ ID NO: 80 sets forth the nucleic acid sequence of a human HAO 1 gene TALEN nuclease recognition sequence spacer (antisense strand).

SEQ ID NO: 81 sets forth the nucleic acid sequence of a human HAO 1 gene TALEN nuclease recognition sequence spacer (antisense strand).

SEQ ID NO: 82 sets forth the nucleic acid sequence of a human HAO 1 gene TALEN nuclease recognition sequence spacer (antisense strand).

SEQ ID NO: 83 sets forth the nucleic acid sequence of a human HAO 1 gene TALEN nuclease recognition sequence spacer (antisense strand).

SEQ ID NO: 84 sets forth the nucleic acid sequence of a human HAO 1 gene TALEN nuclease recognition sequence spacer (antisense strand).

SEQ ID NO: 85 sets forth the nucleic acid sequence of a human HAO 1 gene TALEN nuclease recognition sequence spacer (antisense strand).

SEQ ID NO: 86 sets forth the nucleic acid sequence of a human HAO 1 gene TALEN nuclease recognition sequence spacer (antisense strand).

SEQ ID NO: 87 sets forth the nucleic acid sequence of a human HAO 1 gene TALEN nuclease recognition sequence spacer (antisense strand).

SEQ ID NO: 88 sets forth the nucleic acid sequence of a human HAO 1 gene TALEN nuclease recognition sequence spacer (antisense strand).

SEQ ID NO: 89 sets forth the nucleic acid sequence of a human HAO 1 gene TALEN nuclease recognition sequence spacer (antisense strand).

SEQ ID NO: 90 sets forth the nucleic acid sequence of a human HAO 1 gene TALEN nuclease recognition sequence spacer (antisense strand).

SEQ ID NO: 91 sets forth the nucleic acid sequence of a human HAO 1 gene TALEN nuclease recognition sequence spacer (antisense strand).

SEQ ID NO: 92 sets forth the nucleic acid sequence of a human HAO 1 gene TALEN nuclease recognition sequence spacer (antisense strand).

SEQ ID NO: 93 sets forth the nucleic acid sequence of a human HAO 1 gene TALEN nuclease recognition sequence spacer (antisense strand).

SEQ ID NO: 94 sets forth the nucleic acid sequence of a human HAO 1 gene TALEN nuclease recognition sequence spacer (antisense strand).

SEQ ID NO: 95 sets forth the nucleic acid sequence of a human HAO 1 gene TALEN nuclease recognition sequence spacer (antisense strand).

SEQ ID NO: 96 sets forth the nucleic acid sequence of a human HAO 1 gene TALEN nuclease recognition sequence spacer (antisense strand).

SEQ ID NO: 97 sets forth the nucleic acid sequence of a human HAO1 gene CRISPR Cas9 recognition sequence (sense strand).

SEQ ID NO: 98 sets forth the nucleic acid sequence of a human HAO1 gene CRISPR Cas9 recognition sequence (sense strand).

SEQ ID NO: 99 sets forth the nucleic acid sequence of a human HAO1 gene CRISPR Cas9 recognition sequence (sense strand).

SEQ ID NO: 100 sets forth the nucleic acid sequence of a human HAO1 gene CRISPR Cas9 recognition sequence (sense strand).

SEQ ID NO: 101 sets forth the nucleic acid sequence of a human HAO1 gene CRISPR Cas9 recognition sequence (sense strand).

SEQ ID NO: 102 sets forth the nucleic acid sequence of a human HAO1 gene CRISPR Cas9 recognition sequence (antisense strand).

SEQ ID NO: 103 sets forth the nucleic acid sequence of a human HAO1 gene CRISPR Cas9 recognition sequence (antisense strand).

SEQ ID NO: 104 sets forth the nucleic acid sequence of a human HAO1 gene CRISPR Cas9 recognition sequence (antisense strand).

SEQ ID NO: 105 sets forth the nucleic acid sequence of a human HAO1 gene CRISPR Cas9 recognition sequence (antisense strand).

SEQ ID NO: 106 sets forth the nucleic acid sequence of a human HAO1 gene CRISPR Cpfl recognition sequence (sense strand).

SEQ ID NO: 107 sets forth the nucleic acid sequence of a human HAO1 gene CRISPR Cpfl recognition sequence (sense strand).

SEQ ID NO: 108 sets forth the nucleic acid sequence of a human HAO1 gene CRISPR Cpfl recognition sequence (sense strand).

SEQ ID NO: 109 sets forth the nucleic acid sequence of a human HAO1 gene CRISPR Cpfl recognition sequence (sense strand).

SEQ ID NO: 110 sets forth the nucleic acid sequence of a human HAO1 gene CRISPR Cpfl recognition sequence (sense strand).

SEQ ID NO: 111 sets forth the nucleic acid sequence of a human HAO1 gene CRISPR Cpfl recognition sequence (sense strand).

SEQ ID NO: 112 sets forth the nucleic acid sequence of a human HAO1 gene CRISPR Cpfl recognition sequence (antisense strand).

SEQ ID NO: 113 sets forth the nucleic acid sequence of a human HAO1 gene CRISPR Cpfl recognition sequence (antisense strand).

SEQ ID NO: 114 sets forth the nucleic acid sequence of a human HAO1 gene CRISPR Cpfl recognition sequence (antisense strand).

SEQ ID NO: 115 sets forth the nucleic acid sequence of a human HAO1 gene CRISPR Cpfl recognition sequence (antisense strand).

SEQ ID NO: 116 sets forth the nucleic acid sequence of a target forward primer.

SEQ ID NO: 117 sets forth the nucleic acid sequence of a target reverse primer.

SEQ ID NO: 118 sets forth the nucleic acid sequence of a target probe.

SEQ ID NO: 119 sets forth the nucleic acid sequence of a reference forward primer.

SEQ ID NO: 120 sets forth the nucleic acid sequence of a reference reverse primer.

SEQ ID NO: 121 sets forth the nucleic acid sequence of a reference probe.

SEQ ID NO: 122 sets forth the nucleic acid sequence of a reference forward primer.

SEQ ID NO: 123 sets forth the nucleic acid sequence of a reference reverse primer.

SEQ ID NO: 124 sets forth the nucleic acid sequence of a reference probe.

SEQ ID NO: 125 sets forth the nucleic acid sequence of the forward primer 3963_mHAO1-2F.100.

SEQ ID NO: 126 sets forth the nucleic acid sequence of the reverse primer 3965_mHAO1-2R.119.

SEQ ID NO: 127 sets forth the amino acid sequence of a polypeptide linker.

SEQ ID NO: 128 sets forth the amino acid sequence of the HAO 1-2L.30S19 meganuclease.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference. The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “exogenous” or “heterologous” in reference to a nucleotide sequence or amino acid sequence is intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the terms “nuclease” and “endonuclease” are used interchangeably to refer to naturally-occurring or engineered enzymes which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to SEQ ID NO: 127, which sets forth residues 154-195 of any one of SEQ ID NOs: 7, 8, 9, or 10. In some embodiments, a linker may have an amino acid sequence comprising SEQ ID NO:127, which sets forth residues 154-195 of any one of SEQ ID NOs: 7, 8, 9, or 10.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12^{th} and 13^{th} amino acids. These two positions, referred to as the repeat variable dipeptide (RVD), are highly variable and show a strong correlation with specific nucleotide recognition. Each base pair in the DNA target sequence is contacted by a single TAL repeat, with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein which binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, EndI, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the term “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) which bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpfl. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation.

As used herein, a “template nucleic acid” refers to a nucleic acid sequence that is desired to be inserted into a cleavage site within a cell's genome.

As used herein, with respect to a protein, the term “recombinant” or “engineered” means having an altered amino acid sequence as a result of the application of genetic engineering techniques to nucleic acids which encode the protein, and cells or organisms which express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from engineered or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized and bound by the I-TevI domain, followed by a non-specific spacer 4-16 basepairs in length, followed by a second sequence 16-22 bp in length that is recognized and bound by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a Cpfl CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize and bind to an 18-bp recognition sequence comprising a pair of nine basepair “half-sites” separated by a 2-10 basepair “spacer” sequence, and cleavage by the nuclease (i.e., a left zinc finger and a right zinc finger pair) creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the term “DNA-binding affinity” or “binding affinity” means the tendency of a nuclease to non-covalently associate with a reference DNA molecule (e.g., a recognition sequence or an arbitrary sequence). Binding affinity is measured by a dissociation constant, K_{d}. As used herein, a nuclease has “altered” binding affinity if the K_{d} of the nuclease for a reference recognition sequence is increased or decreased by a statistically significant percent change relative to a reference nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, a nuclease has “altered” specificity if it binds to and cleaves a recognition sequence which is not bound to and cleaved by a reference nuclease (e.g., a wild-type) under physiological conditions, or if the rate of cleavage of a recognition sequence is increased or decreased by a biologically significant amount (e.g., at least 2×, or 2×-10×) relative to a reference nuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function.

As used herein, the term “reduced” refers to any reduction of the recited measurement (e.g., serum oxalate values, urinary oxalate levels, or peroxisomal localization of HAO1 protein) when compared to a control. Such a reduction may be up to 5%, 10%, 20%, 30%, 40%, 50%, 60%, 70%, 80%, 90%, 95%, or up to 100%.

As used herein, “homology arms” or “sequences homologous to sequences flanking a meganuclease cleavage site” refer to sequences flanking the 5′ and 3′ ends of a nucleic acid molecule which promote insertion of the nucleic acid molecule into a cleavage site generated by a meganuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=2; match reward=1; and mismatch penalty=3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the term “recognition half-site,” “recognition sequence half-site,” or simply “half-site” means a nucleic acid sequence in a double-stranded DNA molecule which is recognized and bound by a monomer of a homodimeric or heterodimeric meganuclease, or by one subunit of a single-chain meganuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs:7, 8, 9, or 10. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of any one of SEQ ID NOs:7, 8, 9, or 10. In other embodiments, variable residues within a hypervariable region correspond to one or more of positions 215, 217, 219, 221, 223, 224, 229, 231, 233, 235, 237, 259, 261, 266, and 268 of any one of SEQ ID NOs: 7, 8, 9, or 10. In particular embodiments, variable residues can include one or more of positions 239 and 241 of SEQ ID NO: 9. In some embodiments, variable residues can include one or more of positions 239, 241, 262, 263, 264, and 265 of SEQ ID NO: 10.

As used herein, “HAO1 gene” refers to a gene encoding a polypeptide having 2-hydroxyacid oxidase activity, particularly the hydroxyacid oxidase 1 polypeptide, which is also referred to as glycolate oxidase. An HAO1 gene can include a human HAO1 gene (NCBI Accession No.: NM_017545.2; NP_060015.1; Gene ID: 54363; SEQ ID NO: 3); cynomolgus monkey (Macaca, mulatta) HAO1 (NCBI Accession No.: XM_001116000.2, XP_001116000.1); and mouse (Mus musculus) HAO1, (NCBI Accession No.: NM_010403.2; NP_034533.1). Additional examples of HAO1 mRNA sequences are readily available using publicly available databases, e.g., GenBank, UniProt, OMIM, and the Macaca genome project web site. The term HAO1 also refers to naturally occurring DNA sequence variations of the HAO1 gene, such as a single nucleotide polymorphism (SNP) in the HAO1 gene. Exemplary SNPs may be found through the publically accessible National Center for Biotechnology Information dbSNP Short Genetic Variations database.

As used herein, the term “HAO1 polypeptide” refers to a polypeptide encoded by an HAO1 gene. The HAO1 polypeptide is also known as glycolate oxidase.

As used herein, the term “peroxisomal targeting signal” refers to an amino acid motif that is essential for peroxisomal localization of a polypeptide gene product (e.g., HAO1 polypeptide). In the case of an HAO1 polypeptide, the peroxisomal targeting signal comprises a SKI motif positioned at the C-terminus of the polypeptide. The SKI motif is encoded by codons within exon 8 of the HAO1 gene.

As used herein, the term “disrupts coding of said peroxisomal targeting signal” refers to any nucleotide modification (e.g., insertion, deletion, or substitution) within a gene (e.g., a HAO1 gene) that prevents expression, wholly or in part, of a peroxisomal targeting signal or otherwise results in an amino acid change in the encoded peptide motif such that the SKI motif is no longer capable of signaling transport of protein to the peroxisome.

As used herein, the term “primary hyperoxaluria type 1” or “PH1” refers to a autosomal recessive disorder caused by a mutation in the gene encoding alanine glyoxylate aminotransferase (AGT), a peroxisomal vitamin B6-dependent enzyme, in which the mutation results in decreased conversion of glyoxylate to glycine and consequently, an increase in conversion of glyoxylate to oxalate.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. As used herein, a “vector” can also refer to a viral vector. Viral vectors can include, without limitation, retroviral vectors, lentiviral vectors, adenoviral vectors, and adeno-associated viral vectors (AAV).

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the terms “treatment” or “treating a subject” refers to the administration of an engineered nuclease of the invention, or a nucleic acid encoding an engineered nuclease of the invention, to a subject having primary hyperoxaluria type 1. Such treatment results in a modification of the HAO1 gene sufficient to reduce oxalate levels in the subject, and either partial or complete relief of one or more symptoms of primary hyperoxaluria in the subject. In some aspects, an engineered nuclease of the invention or a nucleic acid encoding the same is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In some specific embodiments, an effective amount of the engineered meganuclease comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding the engineered nuclease. In specific embodiments, an effective amount of an engineered nuclease, nucleic acid encoding an engineered nuclease, or pharmaceutical composition comprising an engineered nuclease or nucleic acid encoding an engineered nuclease disclosed herein, reduces at least one symptom of a disease in a subject (e.g., a modification of the HAO1 gene sufficient to reduce oxalate levels in the subject, and either partial or complete relief of one or more symptoms of primary hyperoxaluria in the subject).

The term “gc/kg” or “gene copies/kilogram” refers to the number of copies of a nucleic acid encoding an engineered meganuclease described herein per weight in kilograms of a subject that is administered the nucleic acid encoding the engineered meganuclease.

The term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

The present invention is based, in part, on the hypothesis that engineered nucleases can be designed to bind and cleave recognition sequences found within a HAO1 gene (e.g., the human HAO1 gene; SEQ ID NO: 3), particularly within or adjacent to exon 8. Surprisingly, targeting nucleases to exon 8 of HAO1, which is the most downstream coding sequence of the HAO1 gene, is an effective approach to disrupt the HAO1-catalyzed conversion of glycolate to glyoxylate. Exon 8 is highly conserved across species, with only a one base pair difference between the human, rhesus monkey, and mouse HAO1 genes Importantly, the present approach generates a mutation in exon 8 that disrupts the coding of the C-terminal SKI motif. The SKI motif is a non-canonical peroxisomal targeting signal (PTS) that is essential for transport of the HAO1 protein into the peroxisome, where the HAO1 protein catalyzes the conversion of glycolate to glyoxylate. The absence of the SKI motif results in an HAO1 protein that is largely intact and potentially active, but not localized to the peroxisome. As a result, levels of the glycolate substrate in cells expressing the modified HAO1 gene will be elevated, while levels of glyoxylate in the peroxisome, and oxalate in the cytoplasm, will be reduced. This approach is effective because glycolate is a highly soluble small molecule that can be eliminated at high concentrations in the urine without affecting the kidney. The effectiveness of this approach is demonstrated herein using in vitro models and in vivo studies, as further outlined in the Examples.

Thus, the present invention encompasses engineered nucleases that bind and cleave a recognition sequence within or adjacent to exon 8 (e.g., SEQ ID NO: 4) of a HAO1 gene (e.g., the human HAO1 gene; SEQ ID NO: 3). The present invention further provides methods comprising the delivery of an engineered protein, or nucleic acids encoding an engineered nuclease, to a eukaryotic cell in order to produce a genetically-modified eukaryotic cell. Further, the present invention provides pharmaceutical compositions, methods for treatment of primary hyperoxaluria, and methods for reducing serum oxalate levels which utilize an engineered nuclease having specificity for a recognition sequence positioned within or adjacent to exon 8 of a HAO1 gene.

2.2 Nucleases for Recognizing and Cleaving Recognition Sequences within a HAO1 Gene

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest.

Thus, in different embodiments, a variety of different types of nucleases are useful for practicing the invention. In one embodiment, the invention can be practiced using engineered recombinant meganucleases. In another embodiment, the invention can be practiced using a CRISPR system nuclease or CRISPR system nickase. Methods for making CRISPR and CRISPR Nickase systems that recognize and bind pre-determined DNA sites are known in the art, for example Ran, et al. (2013) Nat Protoc. 8:2281-308. In another embodiment, the invention can be practiced using TALENs or Compact TALENs. Methods for making TALE domains that bind to pre-determined DNA sites are known in the art, for example Reyon et al. (2012) Nat Biotechnol. 30:460-5. In another embodiment, the invention can be practiced using zinc finger nucleases (ZFNs). In a further embodiment, the invention can be practiced using megaTALs.

In particular embodiments, the nucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

In some examples, engineered meganucleases of the invention have been engineered to bind and cleave an HAO 1-2 recognition sequence (SEQ ID NO: 5). The HAO 1-2 recognition sequence is positioned within exon 8 of the HAO1 gene. Such engineered meganucleases are collectively referred to herein as “HAO 1-2 meganucleases.”

Engineered meganucleases of the invention comprise a first subunit, comprising a first hypervariable (HVR1) region, and a second subunit, comprising a second hypervariable (HVR2) region. Further, the first subunit binds to a first recognition half-site in the recognition sequence (e.g., the HAO1 half-site), and the second subunit binds to a second recognition half-site in the recognition sequence (e.g., the HAO2 half-site). In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit. Exemplary HAO 1-2 meganucleases of the invention are provided in SEQ ID NOs: 7, 8, 9, or 10 and summarized in Table 1.

TABLE 1
Exemplary engineered meganucleases engineered to bind and
cleave the HAO 1-2 recognition sequence (SEQ ID NO: 5)

| | AA | HAO1 | HAO1 | *HAO1 | HAO2 | HAO2 | *HAO2 |
|---|---|---|---|---|---|---|---|
| | SEQ | Subunit | Subunit | Subunit | Subunit | Subunit | Subunit |
| Meganuclease | ID | Residues | SEQ ID | % | Residues | SEQ ID | % |
| HAO 1-2L.30 | 7 | 7-153 | 11 | 100 | 198-344 | 15 | 100 |
| HAO 1-2L.5 | 8 | 7-153 | 12 | 98.64 | 198-344 | 16 | 99.32 |
| HAO 1-2L.285 | 9 | 7-153 | 13 | 98.64 | 198-344 | 17 | 97.28 |
| HAO 1-2L.338 | 10 | 7-153 | 14 | 98.64 | 198-344 | 18 | 94.56 |

*“HAO 1 Subunit %” and “HAO 2 Subunit %” represent the amino acid sequence identity between the HAO1-binding and HAO2-binding subunit regions of each meganuclease and the HAO1-binding and HAO2-binding subunit regions, respectively, of the HAO 1-2L.30 meganuclease.

In certain embodiments of the invention, the engineered meganuclease binds and cleaves a recognition sequence comprising SEQ ID NO: 5 within an HAO1 gene, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region.

In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 7. In some such embodiments, the HVR1 region comprises one or more residues corresponding to residues 24, 26, 28, 30, 32, 33, 38, 40, 42, 44, 46, 68, 70, 75, and 77 of SEQ ID NO: 7. In some such embodiments, the HVR1 region comprises residues corresponding to residues 24, 26, 28, 30, 32, 33, 38, 40, 42, 44, 46, 68, 70, 75, and 77 of SEQ ID NO: 7. In some such embodiments, the HVR1 region comprises Y, R, K, or D at a residue corresponding to residue 66 of SEQ ID NO: 7. In some such embodiments, the HVR1 region comprises residues 24-79 of SEQ ID NO: 7. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 7. In some such embodiments, the HVR2 region comprises one or more residues corresponding to residues 215, 217, 219, 221, 223, 224, 229, 231, 233, 235, 237, 259, 261, 266, and 268 of SEQ ID NO: 7. In some such embodiments, the HVR2 region comprises residues corresponding to residues 215, 217, 219, 221, 223, 224, 229, 231, 233, 235, 237, 259, 261, 266, and 268 of SEQ ID NO: 7. In some such embodiments, the HVR2 region comprises Y, R, K, or D at a residue corresponding to residue 257 of SEQ ID NO: 7. In some such embodiments, the HVR2 region comprises residues 215-270 of SEQ ID NO: 7. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 7, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 7. In some such embodiments, the first subunit comprises G, S, or A at a residue corresponding to residue 19 of SEQ ID NO: 7. In some such embodiments, the first subunit comprises E, Q, or K at a residue corresponding to residue 80 of SEQ ID NO: 7. In some such embodiments, the second subunit comprises G, S, or A at a residue corresponding to residue 210 of SEQ ID NO: 7. In some such embodiments, the second subunit comprises E, Q, or K at a residue corresponding to residue 271 of SEQ ID NO: 7. In some such embodiments, the first subunit comprises a residue corresponding to residue 80 of SEQ ID NO: 7. In some such embodiments, the second subunit comprises a residue corresponding to residue 271 of SEQ ID NO: 7. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 7. In some such embodiments, the engineered meganuclease comprises the amino acid sequence of SEQ ID NO: 7.

In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 8. In some such embodiments, the HVR1 region comprises one or more residues corresponding to residues 24, 26, 28, 30, 32, 33, 38, 40, 42, 44, 46, 68, 70, 75, and 77 of SEQ ID NO: 8. In some such embodiments, the HVR1 region comprises residues corresponding to residues 24, 26, 28, 30, 32, 33, 38, 40, 42, 44, 46, 68, 70, 75, and 77 of SEQ ID NO: 8. In some such embodiments, the HVR1 region comprises Y, R, K, or D at a residue corresponding to residue 66 of SEQ ID NO: 8. In some such embodiments, the HVR1 region comprises residues 24-79 of SEQ ID NO: 8. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 8. In some such embodiments, the HVR2 region comprises one or more residues corresponding to residues 215, 217, 219, 221, 223, 224, 229, 231, 233, 235, 237, 259, 261, 266, and 268 of SEQ ID NO: 8. In some such embodiments, the HVR2 region comprises residues corresponding to residues 215, 217, 219, 221, 223, 224, 229, 231, 233, 235, 237, 259, 261, 266, and 268 of SEQ ID NO: 8. In some such embodiments, the HVR2 region comprises Y, R, K, or D at a residue corresponding to residue 257 of SEQ ID NO: 8. In some such embodiments, the HVR2 region comprises residues 215-270 of SEQ ID NO: 8. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 8, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 8. In some such embodiments, the first subunit comprises G, S, or A at a residue corresponding to residue 19 of SEQ ID NO: 8. In some such embodiments, the first subunit comprises E, Q, or K at a residue corresponding to residue 80 of SEQ ID NO: 8. In some such embodiments, the second subunit comprises G, S, or A at a residue corresponding to residue 210 of SEQ ID NO: 8. In some such embodiments, the second subunit comprises E, Q, or K at a residue corresponding to residue 271 of SEQ ID NO: 8. In some such embodiments, the first subunit comprises a residue corresponding to residue 80 of SEQ ID NO: 8. In some such embodiments, the second subunit comprises a residue corresponding to residue 271 of SEQ ID NO: 8. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 8. In some such embodiments, the engineered meganuclease comprises the amino acid sequence of SEQ ID NO: 8.

In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 9. In some such embodiments, the HVR1 region comprises one or more residues corresponding to residues 24, 26, 28, 30, 32, 33, 38, 40, 42, 44, 46, 68, 70, 75, and 77 of SEQ ID NO: 9. In some such embodiments, the HVR1 region comprises residues corresponding to residues 24, 26, 28, 30, 32, 33, 38, 40, 42, 44, 46, 68, 70, 75, and 77 of SEQ ID NO: 9. In some such embodiments, the HVR1 region comprises Y, R, K, or D at a residue corresponding to residue 66 of SEQ ID NO: 9. In some such embodiments, the HVR1 region comprises residues 24-79 of SEQ ID NO: 9. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 9. In some such embodiments, the HVR2 region comprises one or more residues corresponding to residues 215, 217, 219, 221, 223, 224, 229, 231, 233, 235, 237, 259, 261, 266, and 268 of SEQ ID NO: 9. In some such embodiments, the HVR2 region comprises residues corresponding to residues 215, 217, 219, 221, 223, 224, 229, 231, 233, 235, 237, 259, 261, 266, and 268 of SEQ ID NO: 9. In some such embodiments, the HVR2 region comprises residues corresponding to residues 239 and 241 of SEQ ID NO: 9. In some such embodiments, the HVR2 region comprises Y, R, K, or D at a residue corresponding to residue 257 of SEQ ID NO: 9. In some such embodiments, the HVR2 region comprises residues 215-270 of SEQ ID NO: 9. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 9, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 9. In some such embodiments, the first subunit comprises G, S, or A at a residue corresponding to residue 19 of SEQ ID NO: 9. In some such embodiments, the first subunit comprises E, Q, or K at a residue corresponding to residue 80 of SEQ ID NO: 9. In some such embodiments, the second subunit comprises G, S, or A at a residue corresponding to residue 210 of SEQ ID NO: 9. In some such embodiments, the second subunit comprises E, Q, or K at a residue corresponding to residue 271 of SEQ ID NO: 9. In some such embodiments, the first subunit comprises a residue corresponding to residue 80 of SEQ ID NO: 9. In some such embodiments, the second subunit comprises a residue corresponding to residue 271 of SEQ ID NO: 9. In some such embodiments, the second subunit comprises a residue corresponding to residue 330 of SEQ ID NO: 9. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 9. In some such embodiments, the engineered meganuclease comprises the amino acid sequence of SEQ ID NO: 9.

In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 10. In some such embodiments, the HVR1 region comprises one or more residues corresponding to residues 24, 26, 28, 30, 32, 33, 38, 40, 42, 44, 46, 68, 70, 75, and 77 of SEQ ID NO: 10. In some such embodiments, the HVR1 region comprises residues corresponding to residues 24, 26, 28, 30, 32, 33, 38, 40, 42, 44, 46, 68, 70, 75, and 77 of SEQ ID NO: 10. In some such embodiments, the HVR1 region comprises Y, R, K, or D at a residue corresponding to residue 66 of SEQ ID NO: 10. In some such embodiments, the HVR1 region comprises residues 24-79 of SEQ ID NO: 10. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 10. In some such embodiments, the HVR2 region comprises one or more residues corresponding to residues 215, 217, 219, 221, 223, 224, 229, 231, 233, 235, 237, 259, 261, 266, and 268 of SEQ ID NO: 10. In some such embodiments, the HVR2 region comprises residues corresponding to residues 215, 217, 219, 221, 223, 224, 229, 231, 233, 235, 237, 259, 261, 266, and 268 of SEQ ID NO: 10. In some such embodiments, the HVR2 region comprises residues corresponding to residues 239, 241, 262, 263, 264, and 265 of SEQ ID NO: 10. In some such embodiments, the HVR2 region comprises Y, R, K, or D at a residue corresponding to residue 257 of SEQ ID NO: 10. In some such embodiments, the HVR2 region comprises residues 215-270 of SEQ ID NO: 10. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 10, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 10. In some such embodiments, the first subunit comprises G, S, or A at a residue corresponding to residue 19 of SEQ ID NO: 10. In some such embodiments, the first subunit comprises E, Q, or K at a residue corresponding to residue 80 of SEQ ID NO: 10. In some such embodiments, the second subunit comprises G, S, or A at a residue corresponding to residue 210 of SEQ ID NO: 10. In some such embodiments, the second subunit comprises E, Q, or K at a residue corresponding to residue 271 of SEQ ID NO: 10. In some such embodiments, the first subunit comprises a residue corresponding to residue 80 of SEQ ID NO: 10. In some such embodiments, the second subunit comprises a residue corresponding to residue 271 of SEQ ID NO: 10. In some such embodiments, the second subunit comprises a residue corresponding to residue 330 of SEQ ID NO: 10. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 10. In some such embodiments, the engineered meganuclease comprises the amino acid sequence of SEQ ID NO: 10.

In some embodiments, the engineered nuclease has specificity for a recognition sequence positioned within or adjacent to exon 8 of the HAO1 gene. The recognition sequence can be positioned at any location within or adjacent to exon 8 that disrupts the coding or function of the peroxisomal transport signal. For example, a recognition sequence positioned adjacent to exon 8 can be positioned up to 100 bp, up to 90 bp, up to 80 bp, up to 70 bp, up to 50 bp, up to 40 bp, up to 30 bp, up to 20 bp, up to 10 bp, up to 5 bp, up to 4 bp, up to 3 bp, up to 2 bp, or 1 bp 5′ upstream of exon 8 or 1, 2, 1-3, 1-4, 1-5, 1-6, 1-7, 1-8, 1-9, 1-10 bp, 10-20 bp, 20-30 bp, 30-40 bp, 40-50 bp, 50-60 bp, 60-70 bp, 70-80 bp, 80-90 bp, or 90-100 bp 5′ upstream of exon 8. In certain embodiments, the recognition sequence positioned adjacent to exon 8 is positioned within 10 bp 5′ upstream of exon 8.

In some embodiments, the recognition sequence positioned adjacent to exon 8 is positioned up to 100 bp, up to 90 bp, up to 80 bp, up to 70 bp, up to 50 bp, up to 40 bp, up to 30 bp, up to 20 bp, up to 10 bp, up to 5 bp, up to 4 bp, up to 3 bp, up to 2 bp, or 1 bp 3′ downstream of exon 8 or up to 1, 2, 1-3, 1-4, 1-5, 1-6, 1-7, 1-8, 1-9, 1-10 bp, 10-20 bp, 20-30 bp, 30-40 bp, 40-50 bp, 50-60 bp, 60-70 bp, 70-80 bp, 80-90 bp, or 90-100 bp 3′ downstream of exon 8. In certain embodiments, the recognition sequence positioned adjacent to exon 8 is positioned within 10 bp 3′ downstream of exon 8.

In some embodiments, the modified HAO1 gene comprises an insertion or deletion within exon 8 which disrupts coding or function of the peroxisomal targeting signal.

### 2.3 Methods for Producing Genetically-Modified Cells

The invention provides methods for producing genetically-modified cells using engineered nucleases that bind and cleave recognition sequences found within an HAO1 gene (e.g., the human HAO1 gene; SEQ ID NO: 3). Cleavage at such recognition sequences can allow for NHEJ at the cleavage site or insertion of an exogenous sequence via homologous recombination, thereby disrupting expression of the peroxisomal targeting signal and consequently interfering with localization of the HAO1 protein to the peroxisome. In some embodiments the modified HAO1 polypeptide is not localized to the peroxisome of the genetically-modified eukaryotic cell. Localization the modified HAO1 protein to the peroxisome can be detected using standard methods in the art, e.g., microscopy, e g, immunofluorescence microscopy. See, for instance, Example 5. In specific embodiments, localization of the modified HAO1 polypeptide to the peroxisome is reduced by at least 1%, at least 5%, at least 10%, at least about 20%, at least about 30%, at least about 40%, at least about 50%, at least about 60%, at least about 70%, at least about 80%, at least about 90%, or up to 100% relative to a control.

In some embodiments, disruption of the peroxisomal targeting signal of the HAO1 gene can reduce the conversion of glycolate to glyoxylate. The conversion of glycolate to glyoxylate can be determined by measurements of glycolate and/or glyoxylate levels in the genetically-modified eukaryotic cell relative to a control (e.g., a control cell). For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In specific embodiments, the conversion of glycolate to glyoxylate can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the conversion of glycolate to glyoxylate can be reduced by 1-5%, 5%-10%, 10%-20%, 20%-30%, 30%-40%, 40%-50%, 50%-60%, 70%-80%, 90%-95%, 95%-98%, or up 100% relative to the control.

Oxalate levels can be reduced in a genetically-modified eukaryotic cell relative to a control (e.g., a control cell) by disrupting the peroxisomal targeting signal. For example, the control may be a eukaryotic cell treated with a nuclease that does not target exon 8 of a HAO1 gene, a eukaryotic cell not treated with a nuclease (e.g., treated with PBS or untreated), or a eukaryotic cell prior to treatment with a nuclease of the invention. In some embodiments, the production of oxalate, or oxalate level, can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the production of oxalate can be reduced by 1%-5%, 5%-10%, 10%-20%, 20%-30%, 30%-40%, 40%-50%, 50%-60%, 70%-80%, 90%-95%, 95%-98%, or up to 100% relative to the control. Oxalate levels can be measured in a cell, tissue, organ, blood, or urine, as described elsewhere herein.

In some embodiments, the methods disclosed herein are effective to increase a glycolate/creatinine ratio relative to a reference level. For example, methods disclosed herein can increase the glycolate/creatinine ratio in a urine sample from the subject and/or decrease an oxalate/creatinine ratio in a urine sample from the subject relative to a reference level. In specific embodiments of the method, the reference level is the oxalate/creatinine ratio and/or glycolate/creatinine ratio in a urine sample in a control subject having PH1. The control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the oxalate/creatinine ratio can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the reference level. In some embodiments, the oxalate/creatinine ratio can be reduced by 1%-5%, 5%-10%, 10%-20%, 20%-30%, 30%-40%, 40%-50%, 50%-60%, 70%-80%, 90%-95%, 95%-98%, or up to 100% relative to the reference level.

In some embodiments, the glycolate/creatinine ratio can be increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 100% relative to the reference level. In some embodiments, the glycolate/creatinine ratio can be increased by at least about 2λ-fold, at least about 3λ-fold, at least about 4λ-fold, at least about 5λ-fold, at least about 6λ-fold, at least about 7λ-fold, at least about 8λ-fold, at least about 9λ-fold, or at least about 10λ-fold relative to the reference level.

The methods disclosed herein can be used to decrease the level of calcium precipitates in a kidney of the subject relative to a reference level. The reference level can be the level of calcium precipitates in the kidney of a control subject having PH1. For example, the control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In particular embodiments, the level of calcium precipitates can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the reference level. In some embodiments, the level of calcium precipitates can be reduced by 1%-5%, 5%-10%, 10%-20%, 20%-30%, 30%-40%, 40%-50%, 50%-60%, 70%-80%, 90%-95%, 95%-98%, or up to 100% relative to the reference level.

The methods disclosed herein can be effective to decrease the risk of renal failure in the subject relative to a control subject having PH1. The control subject may be a subject having PH1 treated with a nuclease that does not target exon 8 of a HAO1 gene, a subject having PH1 not treated with a nuclease (e.g., treated with PBS or untreated), or a subject having PH1 prior to treatment with a nuclease of the invention.

In some embodiments, the risk of renal failure can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the reference level. In some embodiments, the risk of renal failure can be reduced by 1%-5%, 5%-10%, 10%-20%, 20%-30%, 30%-40%, 40%-50%, 50%-60%, 70%-80%, 90%-95%, 95%-98%, or up to 100% relative to the reference level.

The invention further provides methods for treating primary hyperoxaluria type I in a subject by administering a pharmaceutical composition comprising a pharmaceutically acceptable carrier and an engineered nuclease of the invention (or a nucleic acid encoding the engineered nuclease).

In each case, the invention includes that an engineered nuclease of the invention can be delivered to and/or expressed from DNA/RNA in cells in vivo that would typically express HAO1 (e.g., cells in the liver (i.e., hepatocytes) or cells in the pancreas).

Engineered nucleases of the invention can be delivered into a cell in the form of protein or, preferably, as a nucleic acid encoding the engineered nuclease. Such nucleic acid can be DNA (e.g., circular or linearized plasmid DNA or PCR products) or RNA (e.g., mRNA).

For embodiments in which the engineered nuclease coding sequence is delivered in DNA form, it should be operably linked to a promoter to facilitate transcription of the nuclease gene. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). An engineered nuclease of the invention can also be operably linked to a synthetic promoter. Synthetic promoters can include, without limitation, the JeT promoter (WO 2002/012514). In specific embodiments, a nucleic acid sequence encoding an engineered meganuclease as disclosed herein can be operably linked to a liver-specific promoter. Examples of liver-specific promoters include, without limitation, human alpha-1 antitrypsin promoter, hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter.

In specific embodiments, a nucleic acid sequence encoding at least one engineered meganuclease is delivered on a recombinant DNA construct or expression cassette. For example, the recombinant DNA construct can comprise an expression cassette (i.e., “cassette”) comprising a promoter and a nucleic acid sequence encoding an engineered meganuclease described herein.

In some embodiments, mRNA encoding the engineered nuclease is delivered to a cell because this reduces the likelihood that the gene encoding the engineered nuclease will integrate into the genome of the cell.

Such mRNA encoding an engineered nuclease can be produced using methods known in the art such as in vitro transcription. In some embodiments, the mRNA is 5′ capped using 7-methyl-guanosine, anti-reverse cap analogs (ARCA) (U.S. Pat. No. 7,074,596), CleanCap® analogs such as Cap 1 analogs (Trilink, San Diego, Calif.), or enzymatically capped using vaccinia capping enzyme or similar. In some embodiments, the mRNA may be polyadenylated. The mRNA may contain various 5′ and 3′ untranslated sequence elements to enhance expression the encoded engineered meganuclease and/or stability of the mRNA itself. Such elements can include, for example, posttranslational regulatory elements such as a woodchuck hepatitis virus posttranslational regulatory element. The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

Purified nuclease proteins can be delivered into cells to cleave genomic DNA, which allows for homologous recombination or non-homologous end-joining at the cleavage site with a sequence of interest, by a variety of different mechanisms known in the art, including those further detailed herein.

In another particular embodiment, a nucleic acid encoding an endonuclease of the invention can be introduced into the cell using a single-stranded DNA template. The single-stranded DNA can further comprise a 5′ and/or a 3′ AAV inverted terminal repeat (ITR) upstream and/or downstream of the sequence encoding the engineered meganuclease. In other embodiments, the single-stranded DNA can further comprise a 5′ and/or a 3′ homology arm upstream and/or downstream of the sequence encoding the engineered meganuclease.

In another particular embodiment, genes encoding an endonuclease of the invention can be introduced into a cell using a linearized DNA template. In some examples, a plasmid DNA encoding an endonuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below. In some embodiments, about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding the engineered nuclease is administered to the subject. In some embodiments, at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a nucleic acid encoding the engineered nuclease is administered to the subject. In some embodiments, about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a nucleic acid encoding the engineered nuclease is administered to the subject. In certain embodiments, about 1×10¹² gc/kg to about 9×10¹³ gc/kg (e.g., about 1×10¹² gc/kg, about 2×10¹² gc/kg, about 3×10¹² gc/kg, about about 6×10¹² gc/kg, about 7×10¹² gc/kg, about 4×10¹² gc/kg, 5×10¹² gc/kg, 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a nucleic acid encoding the engineered nuclease is administered to the subject.

The target tissue(s) for delivery of engineered nucleases of the invention include, without limitation, cells of the liver, such as a hepatocyte cell or preferably a primary hepatocyte, more preferably a human hepatocyte or a human primary hepatocyte, a HepG2.2.15 or a HepG2-hNTCP cell. As discussed, nucleases of the invention can be delivered as purified protein or as RNA or DNA encoding the nuclease. In one embodiment, nuclease proteins, or mRNA, or DNA vectors encoding nucleases, are supplied to target cells (e.g., cells in the liver) via injection directly to the target tissue. Alternatively, nuclease protein, mRNA, DNA, or cells expressing nucleases can be delivered systemically via the circulatory system.

In some embodiments, nuclease proteins, DNA/mRNA encoding nucleases, or cells expressing nuclease proteins are formulated for systemic administration, or administration to target tissues, in a pharmaceutically acceptable carrier in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, proteins/RNA/mRNA/cells are typically admixed with a pharmaceutically acceptable carrier. The carrier must, of course, be acceptable in the sense of being compatible with any other ingredients in the formulation and must not be deleterious to the patient. The carrier can be a solid or a liquid, or both, and can be formulated with the compound as a unit-dose formulation.

In some embodiments, the subject is administered a lipid nanoparticle formulation with about 0.1 mg/kg to about 3 mg/kg of mRNA encoding an engineered nuclease. In some embodiments, the subject is administered a lipid nanoparticle formulation with at least about 0.1 mg/kg, at least about 0.25 mg/kg, at least about 0.5 mg/kg, at least about 0.75 mg/kg, at least about 1.0 mg/kg, at least about 1.5 mg/kg, at least about 2.0 mg/kg, at least about 2.5 mg/kg, or at least about 3.0 mg/kg of mRNA encoding an engineered nuclease. In some embodiments, the subject is administered a lipid nanoparticle formulation within about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of mRNA encoding and engineered nuclease.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins or DNA/mRNA encoding the nucleases are encapsulated within liposomes or complexed using cationic lipids (see, e.g., LIPOFECTAMINE transfection reagent, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, genes encoding a nuclease are introduced into a cell using a viral vector. Such vectors are known in the art and include retroviral vectors, lentiviral vectors, adenoviral vectors, and adeno-associated virus (AAV) vectors (reviewed in Vannucci, et al. (2013 New Microbiol. 36:1-22). Recombinant AAV vectors useful in the invention can have any serotype that allows for transduction of the virus into the cell and insertion of the nuclease gene into the cell genome. For example, in some embodiments, recombinant AAV vectors have a serotype of AAV2, AAV6, AAV8, or AAV9. In some embodiments, the viral vectors are injected directly into target tissues. In alternative embodiments, the viral vectors are delivered systemically via the circulatory system. It is known in the art that different AAV vectors tend to localize to different tissues. In liver target tissues, effective transduction of hepatocytes has been shown, for example, with AAV serotypes 2, 8, and 9 (Sands (2011) Methods Mol. Biol. 807:141-157). AAV vectors can also be self-complementary such that they do not require second-strand DNA synthesis in the host cell (McCarty, et al. (2001) Gene Ther. 8:1248-54).

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a particular embodiment, nuclease genes are operably linked to a promoter that drives gene expression preferentially in the target cells. Examples of liver-specific promoters include, without limitation, human alpha-1 antitrypsin promoter, hybrid liver-specific promoter (hepatic locus control region from ApoE gene (ApoE-HCR) and a liver-specific alpha 1-antitrypsin promoter), human thyroxine binding globulin (TBG) promoter, and apolipoprotein A-II promoter.

Methods and compositions are provided for delivering a nuclease disclosed herein to the liver of a subject. In one embodiment, native hepatocytes, which have been removed from the mammal, can be transduced with a vector encoding the engineered nuclease. Alternatively, native hepatocytes of the subject can be transduced ex vivo with an adenoviral vector, which encodes the engineered nuclease and/or a molecule that stimulates liver regeneration, such as a hepatotoxin. Preferably the hepatotoxin is uPA, and has been modified to inhibit its secretion from the hepatocyte once expressed by the viral vector. In another embodiment, the vector encodes tPA, which can stimulate hepatocyte regeneration de novo. The transduced hepatocytes, which have been removed from the mammal, can then be returned to the mammal, where conditions are provided, which are conducive to expression of the engineered meganuclease. Typically the transduced hepatocytes can be returned to the patient by infusion through the spleen or portal vasculature and administration may be single or multiple over a period of 1 to 5 or more days.

In an in vivo aspect of the methods of the invention, a retroviral, pseudotype, or adenoviral associated vector is constructed, which encodes the engineered nuclease and is administered to the subject. Administration of a vector encoding the engineered nuclease can occur with administration of an adenoviral vector that encodes a secretion-impaired hepatotoxin, or encodes tPA, which stimulates hepatocyte regeneration without acting as a hepatotoxin.

In various embodiments of the methods described herein, the one or more engineered nucleases, polynucleotides encoding such engineered nucleases, or vectors comprising one or more polynucleotides encoding such engineered nucleases, as described herein, can be administered via any suitable route of administration known in the art. Accordingly, the one or more engineered nucleases, polynucleotides encoding such engineered nucleases, or vectors comprising one or more polynucleotides encoding such engineered nucleases, as described herein may be administered by an administration route comprising intravenous, intramuscular, intraperitoneal, subcutaneous, intrahepatic, transmucosal, transdermal, intraarterial, and sublingual. Other suitable routes of administration of the engineered nucleases, polynucleotides encoding such engineered nucleases, or vectors comprising one or more polynucleotides encoding such engineered nucleases may be readily determined by the treating physician as necessary.

In some embodiments, a therapeutically effective amount of an engineered nuclease described herein is administered to a subject in need thereof. As appropriate, the dosage or dosing frequency of the engineered nuclease may be adjusted over the course of the treatment, based on the judgment of the administering physician. Appropriate doses will depend, among other factors, on the specifics of any AAV vector chosen (e.g., serotype, etc.), on the route of administration, on the subject being treated (i.e., age, weight, sex, and general condition of the subject), and the mode of administration. Thus, the appropriate dosage may vary from patient to patient. An appropriate effective amount can be readily determined by one of skill in the art. Dosage treatment may be a single dose schedule or a multiple dose schedule. Moreover, the subject may be administered as many doses as appropriate. One of skill in the art can readily determine an appropriate number of doses. The dosage may need to be adjusted to take into consideration an alternative route of administration or balance the therapeutic benefit against any side effects.

The invention further provides for the introduction of an exogenous nucleic acid into the cell, such that the exogenous nucleic acid sequence is inserted into the HAO1 gene at a nuclease cleavage site. In some embodiments, the exogenous nucleic acid comprises a 5′ homology arm and a 3′ homology arm to promote recombination of the nucleic acid sequence into the cell genome at the nuclease cleavage site.

Exogenous nucleic acids of the invention may be introduced into the cell by any of the means previously discussed. In a particular embodiment, exogenous nucleic acids are introduced by way of a viral vector, such as a lentivirus, retrovirus, adenovirus, or preferably a recombinant AAV vector. Recombinant AAV vectors useful for introducing an exogenous nucleic acid can have any serotype that allows for transduction of the virus into the cell and insertion of the exogenous nucleic acid sequence into the cell genome. In some embodiments, recombinant AAV vectors have a serotype of AAV2, AAV6, AAV8, or AAV9. The recombinant AAV vectors can also be self-complementary such that they do not require second-strand DNA synthesis in the host cell.

In another particular embodiment, an exogenous nucleic acid can be introduced into the cell using a single-stranded DNA template. The single-stranded DNA can comprise the exogenous nucleic acid and, in particular embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, genes encoding a nuclease of the invention and/or an exogenous nucleic acid sequence of the invention can be introduced into the cell by transfection with a linearized DNA template. In some examples, a plasmid DNA encoding an engineered nuclease and/or an exogenous nucleic acid sequence can be digested by one or more restriction enzymes such that the circular plasmid DNA is linearized prior to transfection into the cell.

When delivered to a cell, an exogenous nucleic acid of the invention can be operably linked to any promoter suitable for expression of the encoded polypeptide in the cell, including those mammalian promoters and inducible promoters previously discussed. An exogenous nucleic acid of the invention can also be operably linked to a synthetic promoter. Synthetic promoters can include, without limitation, the JeT promoter (WO 2002/012514).

### 2.4 Pharmaceutical Compositions

In some embodiments, the invention provides a pharmaceutical composition comprising a pharmaceutically acceptable carrier and engineered nuclease of the invention, or a pharmaceutically acceptable carrier and an isolated polynucleotide comprising a nucleic acid encoding an engineered nuclease of the invention. In other embodiments, the invention provides a pharmaceutical composition comprising a pharmaceutically acceptable carrier and a genetically-modified cell of the invention which can be delivered to a target tissue where the cell can then differentiate into a cell which expresses modified HAO1. In particular, pharmaceutical compositions are provided that comprise a pharmaceutically acceptable carrier and a therapeutically effective amount of a nucleic acid encoding an engineered meganuclease or an engineered meganuclease, wherein the engineered meganuclease has specificity for a recognition sequence within a HAO1 gene (e.g., HAO 1-2; SEQ ID NO: 5).

Pharmaceutical compositions of the invention can be useful for treating a subject having primary hyperoxaluria type I. In some instances, the subject undergoing treatment in accordance with the methods and compositions provided herein can be characterized by a mutation in an AGXT gene. Other indications for treatment include, but are not limited to, the presence of one or more risk factors, including those discussed previously and in the following sections. A subject having PH1 or a subject who may be particularly receptive to treatment with the engineered nucleases herein may be identified by ascertaining the presence or absence of one or more such risk factors, diagnostic, or prognostic indicators. The determination may be based on clinical and sonographic findings, including urine oxalate assessments, enzymology analyses, and/or DNA analyses known in the art (see, e.g., Example 3).

For example, the subject undergoing treatment can be characterized by urinary oxalate levels, e.g., urinary oxalate levels of at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mg of oxalate per 24 hour period, or more. In certain embodiments, the oxalate level is associated with one or more symptoms or pathologies. Oxalate levels may be measured in a biological sample, such as a body fluid including blood, serum, plasma, or urine. Optionally, oxalate is normalized to a standard protein or substance, such as creatinine in urine. In some embodiments, the claimed methods include administration of any of the engineered nucleases described herein to reduce serum or urinary oxalate levels in a subject to undetectable levels, or to less than 1% 2%, 5%, 10%, 20%, 30%, 40%, 50%, 60%, 70%, or 80% of the subject's oxalate levels prior to treatment, within 1, 3, 5, 7, 9, 12, or 15 days.

For example, hyperoxaluria in humans can be characterized by urinary oxalate excretion, e.g., excretion greater than about 40 mg (approximately 440 mol) or greater than about 30 mg per day. Exemplary clinical cutoff levels for urinary oxalate are 43 mg/day (approximately 475 mol) for men and 32 mg/day (approximately 350 μmop for women, for example.

Hyperoxaluria can also be defined as urinary oxalate excretion greater than 30 mg per day per gram of urinary creatinine. Persons with mild hyperoxaluria may excrete at least 30-60 (342-684 mol) or 40-60 (456-684 μmop mg of oxalate per day. Persons with enteric hyperoxaluria may excrete at least 80 mg of urinary oxalate per day (912 mol), and persons with primary hyperoxaluria may excrete at least 200 mg per day (2280 mol).

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, nuclease polypeptides (or DNA/RNA encoding the same or cells expressing the same) are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

Pharmaceutical compositions are provided that comprise a pharmaceutically acceptable carrier and a therapeutically effective amount of: (a) a nucleic acid encoding an engineered nuclease having specificity for a recognition sequence within an HAO1 gene, wherein the engineered nuclease is expressed in a eukaryotic cell in vivo; or (b) an engineered nuclease having specificity for a recognition sequence within an HAO1 gene; wherein the engineered nuclease produces a cleavage site within the recognition sequence and generates a modified HAO1 gene which encodes a modified HAO1 polypeptide, wherein the modified HAO1 polypeptide comprises the amino acids encoded by exons 1-7 of the HAO1 gene but lacks a peroxisomal targeting signal.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the polypeptide, nucleic acid, or vector to elicit a desired response in the individual. As used herein a therapeutically result can refer to a reduction of serum oxalate level, a reduction in urinary oxalate level, an increase in the glycolate/creatinine ratio, a decrease in the oxalate/creatinine ratio, a decrease in calcium precipitates in the kidney, and/or a decrease in the risk of renal failure.

The pharmaceutical compositions described herein can include an effective amount of any engineered nuclease, or a nucleic acid encoding an engineered nuclease of the invention. In some embodiments, the pharmaceutical composition comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding an engineered nuclease. In some embodiments, the pharmaceutical composition comprises at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a nucleic acid encoding an engineered nuclease. In some embodiments, the pharmaceutical composition comprises about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a nucleic acid encoding an engineered nuclease. In certain embodiments, the pharmaceutical composition comprises about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a nucleic acid encoding an engineered nuclease.

In particular embodiments of the invention, the pharmaceutical composition can comprise one or more mRNAs described herein encapsulated within lipid nanoparticles, which are described elsewhere herein.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)[1,3]-dioxolane (DLin-K-C2-DMA; “XTC2”), 2,2-dilinoleyl-4-(3-dimethylaminopropyl)[1,3]-dioxolane (DLin-K-C3-DMA), 2,2-dilinoleyl-4-(4-dimethylaminobutyl)[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-T-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid may comprise from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid may comprise from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-T-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-w-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition may comprise amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Choi 3-β[N-(N′,N′-dimethylmethane) carbamoyl] cholesterol, TC-Chol 3-β-[N-(N′,N′,N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes may contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition which specifically enhances delivery and uptake in the liver, or specifically within hepatocytes.

### 2.5 Methods for Producing Recombinant Viral Vectors

In some embodiments, the invention provides viral vectors (e.g., recombinant AAV vectors) for use in the methods of the invention. Recombinant AAV vectors are typically produced in mammalian cell lines such as HEK-293. Because the viral cap and rep genes are removed from the vector to prevent its self-replication to make room for the therapeutic gene(s) to be delivered (e.g. the meganuclease gene), it is necessary to provide these in trans in the packaging cell line. In addition, it is necessary to provide the “helper” (e.g. adenoviral) components necessary to support replication (Cots et al. (2013), Curr. Gene Ther. 13(5): 370-81). Frequently, recombinant AAV vectors are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAV particles are typically produced (manufactured) in cells, precautions must be taken in practicing the current invention to ensure that the engineered nuclease is not expressed in the packaging cells. Because the viral genomes of the invention may comprise a recognition sequence for the nuclease, any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The nuclease can be placed under the control of a tissue-specific promoter that is not active in the packaging cells. For example, if a viral vector is developed for delivery of (a) meganuclease gene(s) to muscle tissue, a muscle-specific promoter can be used. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). Examples of CNS (neuron)-specific promoters include the NSE, Synapsin, and MeCP2 promoters (Lentz, et al. (2012) Neurobiol Dis. 48:179-88). Examples of liver-specific promoters include albumin promoters (such as Palb), human al-antitrypsin (such as PalAT), and hemopexin (such as Phpx) (Kramer et al., (2003) Mol. Therapy 7:375-85), hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter. Examples of eye-specific promoters include opsin, and corneal epithelium-specific K12 promoters (Martin et al. (2002) Methods (28): 267-75) (Tong et al., (2007) J Gene Med, 9:956-66). These promoters, or other tissue-specific promoters known in the art, are not highly-active in HEK-293 cells and, thus, will not be expected to yield significant levels of meganuclease gene expression in packaging cells when incorporated into viral vectors of the present invention. Similarly, the viral vectors of the present invention contemplate the use of other cell lines with the use of incompatible tissue specific promoters (i.e., the well-known HeLa cell line (human epithelial cell) and using the liver-specific hemopexin promoter). Other examples of tissue specific promoters include: synovial sarcomas PDZD4 (cerebellum), C6 (liver), ASB5 (muscle), PPP1R12B (heart), SLC5A12 (kidney), cholesterol regulation APOM (liver), ADPRHL1 (heart), and monogenic malformation syndromes TP73L (muscle). (Jacox et al., (2010), PLoS One v.5(8):e12274).

Alternatively, the vector can be packaged in cells from a different species in which the nuclease is not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007), J. Biotechnol. 131(2):138-43). A meganuclease under the control of a mammalian promoter is unlikely to be expressed in these cells (Airenne et al. (2013), Mol. Ther. 21(4):739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a meganuclease. Because these introns are not spliced efficiently from pre-mRNA transcripts in insect cells, insect cells will not express a functional meganuclease and will package the full-length genome. In contrast, mammalian cells to which the resulting recombinant AAV particles are delivered will properly splice the pre-mRNA and will express functional meganuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1(11): e57).

The nuclease gene can be operably linked to an inducible promoter such that a small-molecule inducer is required for meganuclease expression. Examples of inducible promoters include the Tet-On system (Clontech; Chen et al. (2015), BMC Biotechnol. 15(1):4)) and the RheoSwitch system (Intrexon; Sowa et al. (2011), Spine, 36(10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the nuclease gene under the control of a promoter that responds to the corresponding transcription factor, the nuclease gene having (a) binding site(s) for the transcription factor; and 2) including the gene encoding the transcription factor in the packaged viral genome. The latter step is necessary because the nuclease will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAV particles are produced in a mammalian cell line that expresses a transcription repressor that prevents expression of the meganuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current invention, packaging cells are transfected/transduced with a vector encoding a transcription repressor and the meganuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene will be repressed only in the packaging cells expressing the repressor and not in target cells or tissues transduced with the resulting recombinant AAV vector.

### 2.6 Engineered Nuclease Variants

Embodiments of the invention encompass the engineered nucleases described herein, and variants thereof. Further embodiments of the invention encompass isolated polynucleotides comprising a nucleic acid sequence encoding the nucleases described herein, and variants of such polynucleotides.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; i.e., the ability to bind and cleave recognition sequences found in an HAO1 gene (e.g., the human HAO1 gene; SEQ ID NO: 3). Such variants may result, for example, from human manipulation. In some embodiments, biologically active variants of a native polypeptide of the embodiments (e.g., SEQ ID NOs: 7, 8, 9, or 10), or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, native subunit, native HVR1, or native HVR2 as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein, such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases of the invention comprise an HVR1 that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of any one of SEQ ID NOs: 7, 8, 9, or 10.

In certain embodiments, engineered meganucleases of the invention comprise an HVR2 that has 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 215-270 of any one of SEQ ID NOs: 7, 8, 9, or 10.

A substantial number of amino acid modifications to the DNA recognition domain of the wild-type I-CreI meganuclease have previously been identified (e.g., U.S. Pat. No. 8,021,867) which, singly or in combination, result in engineered meganucleases with specificities altered at individual bases within the DNA recognition sequence half-site, such that the resulting rationally-designed meganucleases have half-site specificities different from the wild-type enzyme. Table 2 provides potential substitutions that can be made in a engineered meganuclease monomer or subunit to enhance specificity based on the base present at each half-site position (−1 through −9) of a recognition half-site.

TABLE 2

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein.
An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CreI or any one of SEQ ID NOs: 7, 8, 9, or 10 (WO 2009001159), a Y, R, K, or D at a residue corresponding to position 66 of I-CreI or any one of SEQ ID NOs: 7, 8, 9, or 10, and/or an E, Q, or K at a residue corresponding to position 80 of I-CreI or any one of SEQ ID NOs: 7, 8, 9, or 10 (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode an engineered nuclease of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially bind and cleave recognition sequences found within a HAO1 gene (e.g., the human HAO1 gene; SEQ ID NO: 3).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Characterization of Meganucleases Having Specificity for the HAO 1-2 Recognition Sequence

1. Meganucleases that Bind and Cleave the HAO 1-2 Recognition Sequence

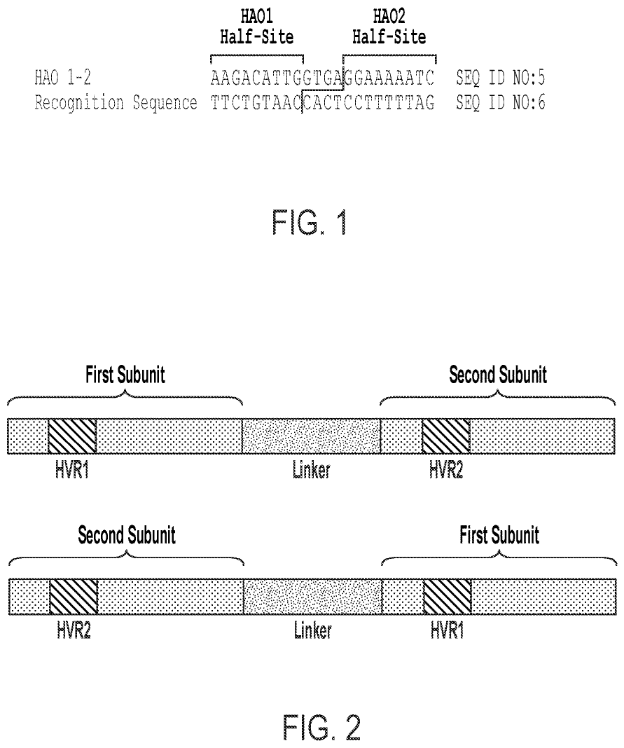

The HAO 1-2 meganucleases described herein (SEQ ID NOs: 7, 8, 9, or 10) were engineered to bind and cleave the HAO 1-2 recognition sequence (SEQ ID NO: 5) which is present within exon 8 of the human, mouse, and rhesus HAO1 genes. Each of these meganucleases comprises an N-terminal nuclease-localization signal derived from SV40, a first meganuclease subunit, a linker sequence, and a second meganuclease subunit. A first subunit in each HAO 1-2 meganuclease binds to the HAO1 recognition half-site of SEQ ID NO: 5, while a second subunit binds to the HAO2 recognition half-site (see, FIG. 1). HAO1-binding subunits and HAO2-binding subunits each comprise a 56 base pair hypervariable region, referred to as HVR1 and HVR2, respectively (see, FIG. 2).

The HVR1 region of each HAO1-binding subunit consists of residues 24-79 of SEQ ID NOs: 7, 8, 9, or 10. HAO1-binding subunits of each nuclease are identical to one another outside of the HVR1 region. The HVR2 region of each HAO2-binding subunit consists of residues 215-270 of SEQ ID NOs: 7, 8, 9, or 10. HAO2-binding subunits of each nuclease are identical to one another outside of the HVR2 region, except at position 271 which can be E, K, or Q, and at position 330, which can be R in SEQ ID NOs: 9 and 10.

### 2. Evaluation of HAO 1-2 Recognition Sequence Cleavage

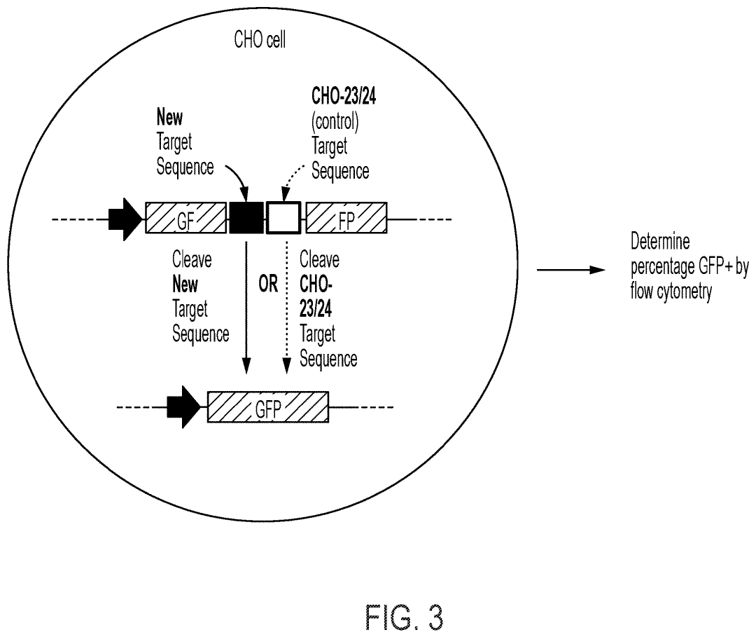

To determine whether HAO 1-2 meganucleases could bind and cleave the HAO 1-2 recognition sequence (SEQ ID NO: 5), the HAO 1-2L.30 (SEQ ID NO: 7) and HAO 1-2L.5 (SEQ ID NO: 8) meganucleases were evaluated using the CHO cell reporter assay previously described (see WO/2012/167192, FIG. 3). To perform the assay, a pair of CHO cell reporter lines were produced which carried a non-functional Green Fluorescent Protein (GFP) gene expression cassette integrated into the genome of the cell. The GFP gene in each cell line was interrupted by a pair of recognition sequences such that intracellular cleavage of either recognition sequence by a meganuclease would stimulate a homologous recombination event resulting in a functional GFP gene. In both cell lines, one of the recognition sequences was derived from the HAO 1-2 gene and the second recognition sequence was specifically recognized and bound by a control meganuclease called “CHO 23/24”. CHO reporter cells comprising the HAO 1-2 recognition sequence (SEQ ID NO: 5) and the CHO 23/24 recognition sequence are referred to herein as “HAO 1-2 cells.”

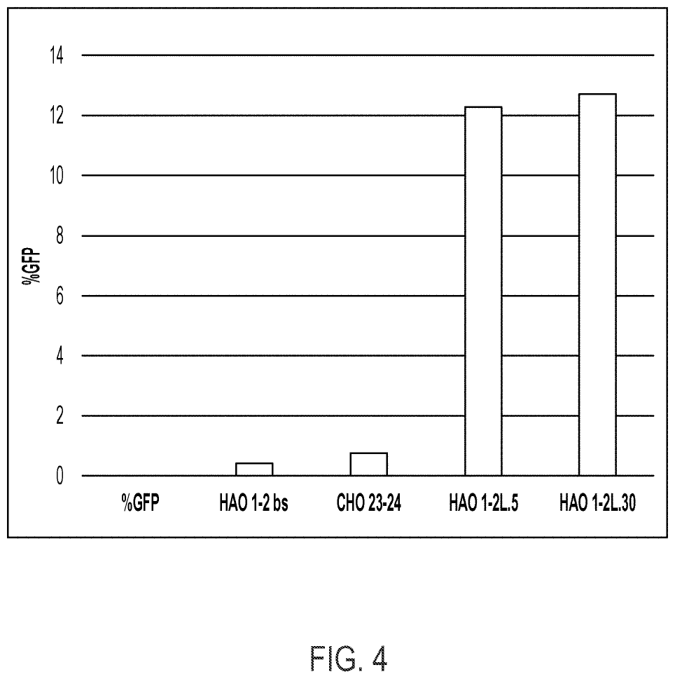

HAO 1-2 cells were transfected with plasmid DNA encoding one of the HAO 1-2 meganucleases (e.g., HAO 1-2L.5 or HAO 1-2L.30) or encoding the CHO 23/34 meganuclease. 4e⁵ CHO cells were transfected with 50 ng of plasmid DNA in a 96-well plate using Lipofectamine 2000 (ThermoFisher) according to the manufacturer's instructions. At 48 hours post-transfection, cells were evaluated by flow cytometry to determine the percentage of GFP-positive cells compared to an untransfected negative control (1-2 bs). Each HAO 1-2 meganuclease was found to produce GFP-positive cells in cell lines comprising the HAO 1-2 recognition sequence at frequencies significantly exceeding the negative control and comparable to or exceeding the CHO 23/24 positive control, indicating that each HAO 1-2 meganuclease was able to efficiently bind and cleave the intended HAO 1-2 recognition sequence in a cell (FIG. 4).

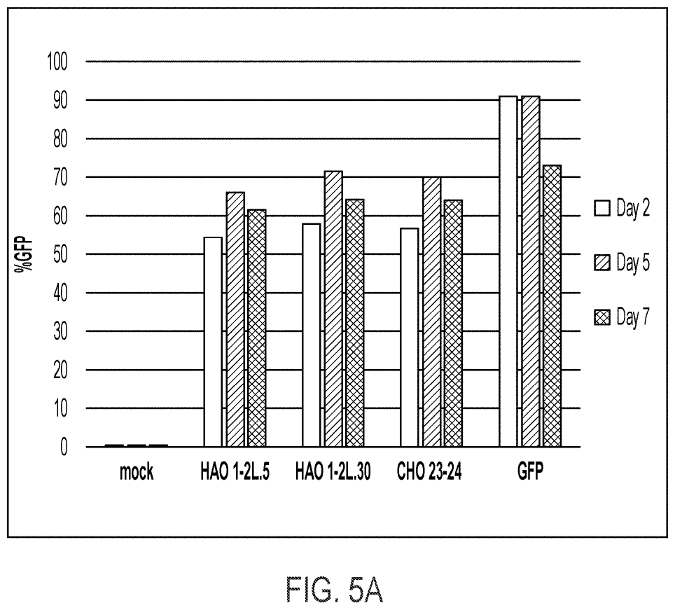

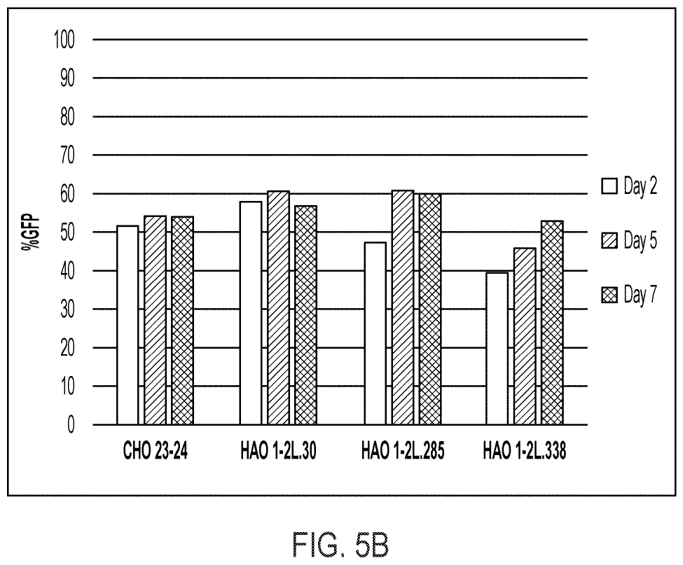

The efficacy of the HAO 1-2L.5 (SEQ ID NO: 8), HAO 1-2L.30 (SEQ ID NO: 7), HAO 1-2L.285 (SEQ ID NO: 9), and HAO 1-2L.338 (SEQ ID NO: 10) meganucleases was also determined in a time-dependent manner 2, 5, and 7 days after introduction of the meganuclease mRNA into HAO 1-2 cells. In this study, HAO 1-2 cells (1.0×10⁶) were electroporated with 1×10⁶ copies of meganuclease mRNA per cell using a BioRad Gene Pulser Xcell according to the manufacturer's instructions. At 48 hours post-transfection, cells were evaluated by flow cytometry to determine the percentage of GFP-positive cells. A CHO 23/24 meganuclease was also included at each time point as a positive control. Each of the meganucleases showed a comparable GFP-positive percentage relative to CHO 23-24 that was stable or increasing over time (FIGS. 5A and 5B). These results demonstrate the ability of these HAO 1-2 meganucleases to bind and cleave the HAO 1-2 recognition sequence in the genome of a cell.

### Example 2

### Digital PCR to Detect Indels Generated by HAO 1-2 Meganucleases

### 1. Methods

These experiments were conducted in in vitro cell based systems to evaluate editing efficiencies of different second-generation HAO 1-2 meganucleases by digital PCR using an indel detection assay. The tested meganucleases included HAO1-2L.30, HAO1-2L.285, HAO1-2L.288, HAO1-2L.298, HAO1-2L.324, HAO1-2L.338, HAO1-2L.360, and HAO1-2L.361. An additional variant meganuclease from the HAO 1-2L.30 meganuclease was generated, which harbored a glycine to serine substitution at residue 19 (HAO 1-2L.30S19).

Cell Culture and Transfection

HepG2 and FL83b cells were cultured and transfected using ThermoFisher's Neon® Transfection system for these experiments. 1×10⁶ HepG2 and 0.5×10⁶ FL83b cells were electroporated with 3 mg of meganuclease RNA using condition 16 and condition 4, respectively. Cells were harvested and genomic DNA isolated at time points indicated in the data.

Digital PCR

Genomic DNA isolation was carried out using the Macherey Nagel Nucleospin Blood QuickPure kit #740569.250 by following manufacturer's instructions. This genomic DNA was used for indel quantification using Bio-Rad's QX200 Droplet Digital PCR system. Two taqman assays were multiplexed in the same reaction, one to detect indels at the HAO 1-2 target site and a reference assay to act as a housekeeping control. The primer and probe sequences for these assays are shown below:

TABLE 3
Primers

| | |
|---|---|
| Target forward | ggtgccagaatgtgaaagt |
| primer | (SEQ ID NO: 116) |
| Target reverse | tggtcaccctctgcaca |
| primer | (SEQ ID NO: 117) |
| Target probe | gacattggtgaggaaaaatcctttgg |
| | (SEQ ID NO: 118) |
| Reference forward | gtgatgatgccagggag |
| primer( Human) | (SEQ ID NO: 119) |
| Reference reverse | ccatcgagttgtcgagc |
| primer (Human) | (SEQ ID NO: 120) |
| Reference probe | gaatgggatcttggtgtcgaatca |
| (Human) | (SEQ ID NO: 121) |
| Reference forward | gtgatgatgccaaggaagc |
| primer (Mouse) | (SEQ ID NO: 122) |
| Reference reverse | gtagctggcaccccatc |
| primer (Mouse) | (SEQ ID NO: 123) |
| Reference probe | tgggatcttggtgtcgaatc |
| (Mouse) | (SEQ ID NO: 124) |

The digital PCR reaction was set up using ddPCR Supermix for Probes (no dUTP) (Catalog #1863024 from Bio-Rad), the target taqman assay (in FAM), the reference taqman assay (in HEX) and HindIII-HF enzyme (NEB Catalog #R3104S) to fragment the genomic DNA. 5000 genome copies of the mock and treated samples were loaded as template in the PCR reaction.

### 2. Results

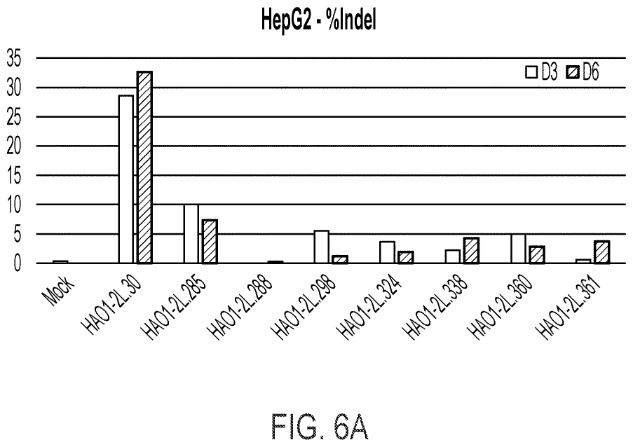

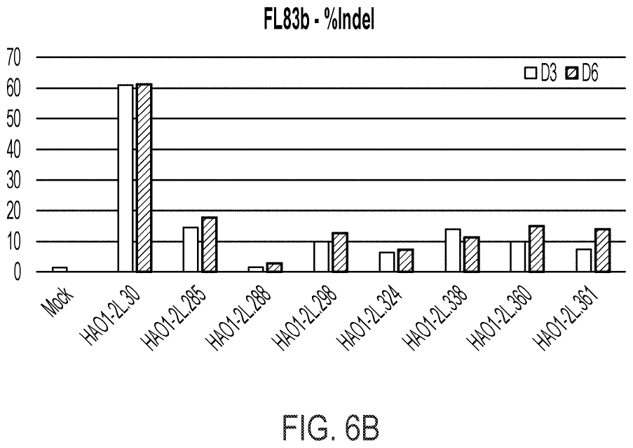

Multiple HAO 1-2 meganucleases were evaluated against the HAO 1-2 target site. These meganucleases included HAO1-2L.30, HAO1-2L.285, HAO1-2L.288, HAO1-2L.298, HAO1-2L.324, HAO1-2L.338, HAO1-2L.360, and HAO1-2L.361. The HAO 1-2L.30 meganuclease was identified to generate three to four fold higher indels in both HepG2 and FL-83b cells using droplet digital PCR (FIGS. 6A and 6B).

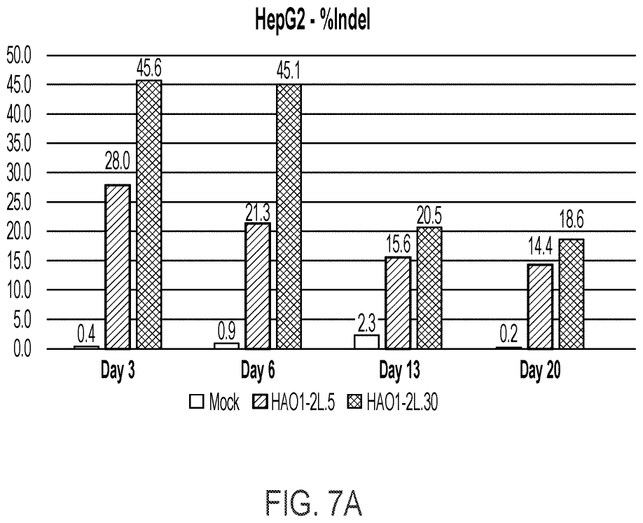

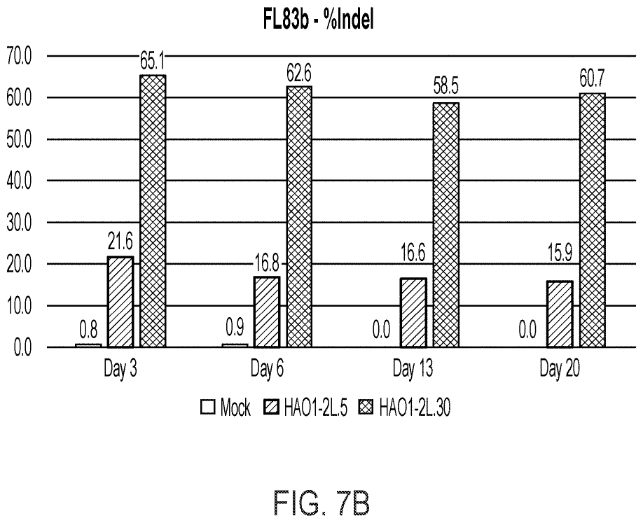

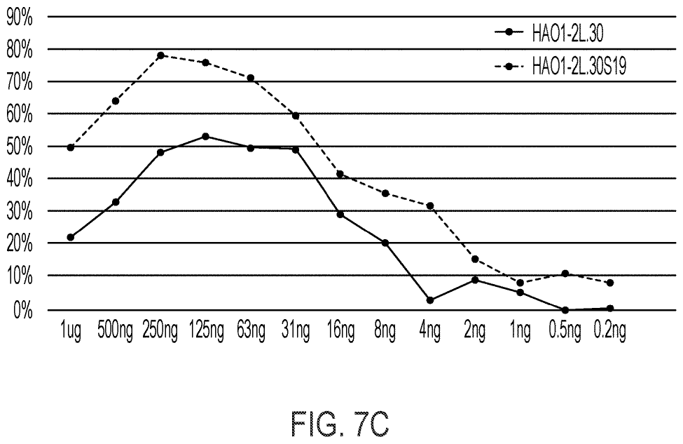

Further, evaluation of HAO 1-2L.30 at different time points showed a decrease in HAO 1-2L.30 activity in human HepG2 cells over time, whereas in mouse liver cells, FL83b, a steady level of indels was observed after single nuclease treatment (FIGS. 7A and 7B). As shown in FIG. 7C the HAO 1-2L.30S19 meganuclease generated significantly higher levels of indel % at every dose tested.

### 3. Conclusions

HAO 1-2L.30 was observed to demonstrate higher HAO1 gene editing in the human and mouse liver lines tested in comparison to other nucleases to the same site. Editing level stayed consistent around 60% in the mouse cell line. Substituting the G19 residue to S19 resulted in even higher levels of editing.

### Example 3

### Mouse Pilot Study: Quantitation of Glycolate in Mouse Serum

### 1. Methods

The HAO 1-2L.30 meganuclease (SEQ ID NO: 7) was tested in C57 mice with the goal of characterizing the effect of nuclease activity against HAO 1-2 on glycolate levels present in mouse serum. 15 C57 mice were injected via tail vein with 5e11VG (viral genomes) of AAV expressing the HAO 1-2L.30 nuclease (pDI TBG HAO 1-2L.30 WPRE). The AAV was manufactured by a commercial vendor using the AAV8 Capsid. Additionally, a control group of 3 C57 mice received a control injection of PBS as a baseline comparator control. Serum was collected for all mice prior to AAV injection. All serum was stored at −80° C. until analysis by LCMS. At weeks 1, 2, 3, and 4 animals from the experimental group were sacrificed with serum collected by terminal bleeds and the livers removed. The final time point, week 5, was extended for 3 additional weeks, with serum collected at week 5, then terminal bleeds at week 8.

Serum glycolate was analyzed and quantified by an external vendor ChemoGenics BioPharma, LLC using LC/MS as described below.

Glycolate Quantification & Method Development

The signal was optimized for each compound by ESI positive or negative ionization mode. A MS2 SIM scan was used to optimize the precursor ion and a product ion analysis was used to identify the best fragment for analysis and to optimize the collision energy.

Calibration and sample details A working dilution of test agent in AcCN at 50 times the final concentration was prepared and serially diluted. Calibration curve ranged from 1.31 μM to 133.3 μM. Samples that fell below 1.31 uM were BLQ. Protein precipitation of serum was done with 3× acetonitrile with deuterated glycolic acid. Analyst 1.62 was used to get the unknown conc from the calibration curve

Analysis

Samples were analyzed by LC/MS/MS using a Sciex API4000 QTRAP mass spectrometer coupled with an Agilent 1200 HPLC and a CTC PAL chilled autosampler, all controlled by Analyst software (ABI). After separation on a C18 reverse phase HPLC column (Agilent, Waters, or equivalent). Mobile phase A was 10 mM ammonium acetate in water. Mobile phase B was 10% AcCN with 10 mM ammonium acetate. The flow rate was 1 mL/min The gradient program included a 0.5 min hold at 2% B (the starting conditions), followed by a gradient to 99% B over 1.5 min and a 1 min hold at 99% B. The column was then returned to starting conditions and equilibrated over 1.0 min

### 2. Results

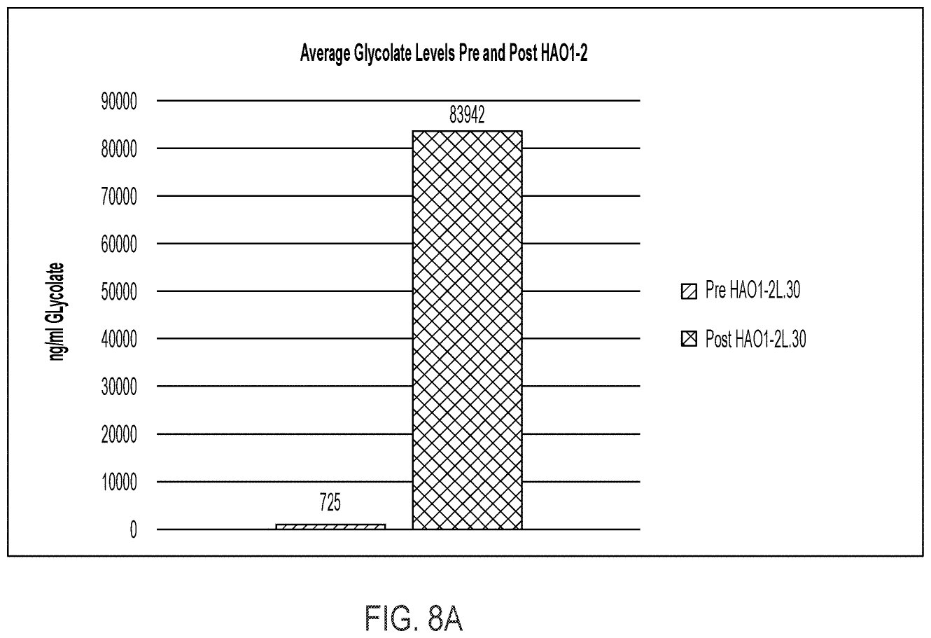

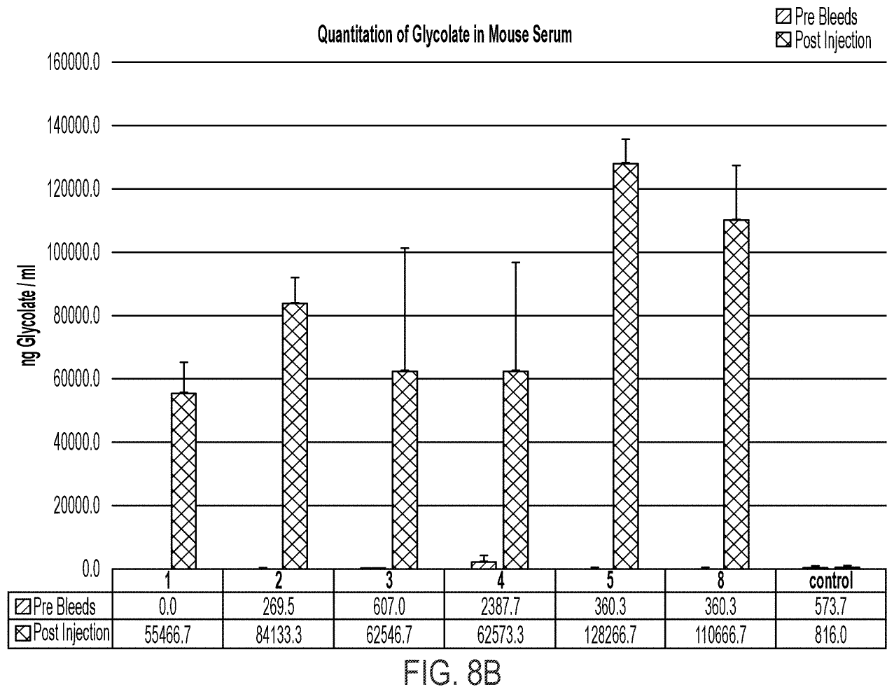

Mice were treated with 5e11VG pDI TBG HAO 1-2L.30 WPRE. As shown in FIG. 8A, the average pre-bleed level of glycolate in all mice in the treated cohort was 725 ng/ml compared to 83,942 ng/ml in AAV-treated mice. Glycolate levels increased 115-fold after injection with AAV encoding the HAO 1-2L.30 meganuclease. As shown in FIG. 8B, elevated levels of glycolate were measured in serum starting at week 1 post injection (>50,000 ng/ml) and continued thru week 8 (>100,000 ng/ml) compared to control mice where no difference was detected in glycolate levels.

### 3. Conclusions

This experiment demonstrated that expression of the HAO 1-2L.30 nuclease in mice had a significant effect on the pathway where HAO1 converts glycolate to glyoxylate. Glycolate levels increased greater than 2 orders of magnitude in mice that were injected pDI TBG HAO 1-2L.30 WPRE. These differences were not noted in PBS control mice. The HAO 1-2L.30 nuclease was also shown to be effective 7 days post injection with significant potency established at this time point when compared to glycolate levels in mice 8 weeks post injection which is slightly increased, less than an order of magnitude.

### Example 4

### Mouse Pilot Study: Quantitation of Indels in Mouse Liver

### 1. Methods

gDNA Isolation from Mouse Livers

gDNA as isolated from mouse livers of Example 3 using the NucleoSpin Tissue kit from Machery-Nagel (ref #740952.250). The protocol was followed per kit manufacturer product manual. Briefly, a small section of liver was placed in a 1.5 ml tube. Lysis was achieved by incubation of the samples in a solution containing SDS and Proteinase K at 65° C. Appropriate conditions for binding of DNA to the silica membrane of the NucleoSpin® Tissue Columns were created by addition of large amounts of chaotropic ions and ethanol to the lysate. The binding process is reversible and specific to nucleic acids. Contaminations are removed by efficient washing with buffer. Pure genomic DNA is finally eluted under low ionic strength conditions in water.

INDEL Analysis by ddPCR

Genomic DNA was used for indel quantification using Bio-Rad's QX200 Droplet Digital PCR system. Two taqman assays were multiplexed in the same reaction, one to detect indels at the HAO 1-2 target site and a reference assay to act as a housekeeping control. The primer and probe sequences for these assays are shown below:

TABLE 4
Primers

| | |
|---|---|
| Target forward primer | ggtgccagaatgtgaaagt |
| | (SEQ ID NO: 116) |
| Target reverse primer | tggtcaccctctgcaca |
| | (SEQ ID NO: 117) |
| Target probe | gacattggtgaggaaaaatcctttgg |
| | (SEQ ID NO: 118) |
| Reference forward | gtgatgatgccaaggaagc |
| primer (Mouse) | (SEQ ID NO: 122) |
| Reference reverse | gtagctggcaccccatc |
| primer (Mouse) | (SEQ ID NO: 123) |
| Reference probe | tgggatcttggtgtcgaatc |
| (Mouse) | (SEQ ID NO: 124) |

Digital PCR reaction was set up using ddPCR Supermix for Probes (no dUTP) (Catalog #1863024 from Bio-Rad), the target taqman assay (in FAM), the reference taqman assay (in HEX) and HindIII-HF enzyme (NEB Catalog #R3104S) to fragment the genomic DNA. 5000 genome copies of the mock and treated samples were loaded as template in the PCR reaction.

PCR Products for Deep Sequencing

Q5 High-Fidelity DNA Polymerase (NEB #M0491) was used with the extracted gDNA from each mouse to PCR amplify a 241 bp amplicon. Gene specific primers were utilized that sat 100 bp upstream and 119 bp downstream of the HAO 1-2 target site (3963_mHAO 1-2F.100, CCTTGGGAAAACGATTACCTGC, SEQ ID NO: 125 and 3965_mHAO 1-2R.119, GAGTTACAGTCTGTGGTCACCC, SEQ ID NO: 126). The PCR products were ran on a 1% agarose gel and the 241 bp band was extracted from the gel using NucleoSpin® Gel and PCR Clean-up from Macherey-Nagel (#740609.10) as directed by the kit manual.

Deep Sequencing

Illumina compatible sequencing libraries were generated using NEBNext Ultra DNA Library Prep Kit for Illumina (NEB, Ipswitch, Mass., USA). Paired-end sequencing data was generated for each library using a MiSeq (Illumina, San Diego, Calif., USA). FastQ reads were joined using Flash and aligned with the reference sequence using BWA-MEM. SAM files were analyzed for insertions or deletions occurring within the specified range using a custom script.

INDEL Analysis by Sanger Sequencing A portion of the 241 bp PCR product obtained was ligated into cloning shuttle vector and transformed into E. coli. Transformants were plated on agar plates and incubated overnight. 41 colonies were picked and used as template for colony PCR using M13 F and M13 reverse primers. Unpurified PCR products were sent to a commercial vendor for sequencing with M13 F and M13 R primers. SnapGene Software was used to analyze the DNA sequence of these PCR products.

### 2. Results

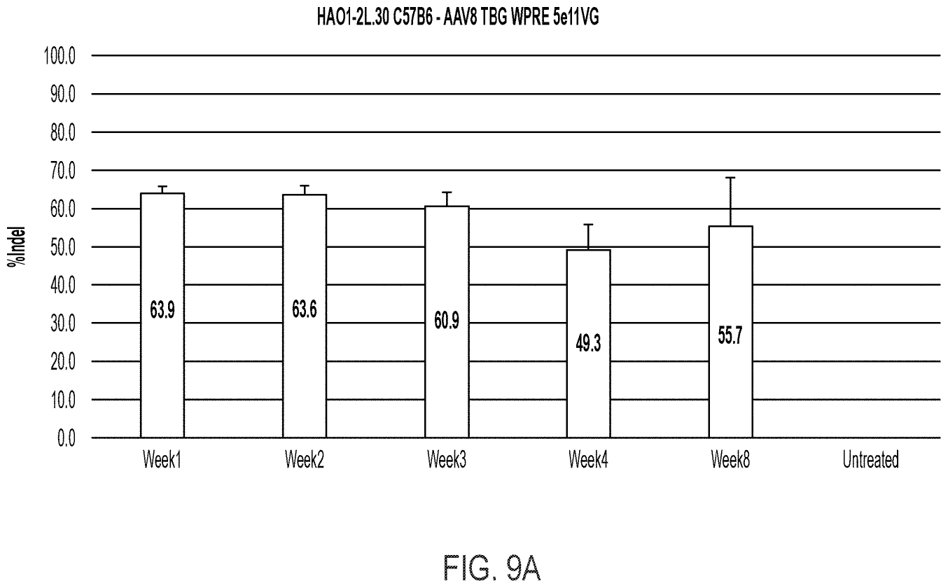

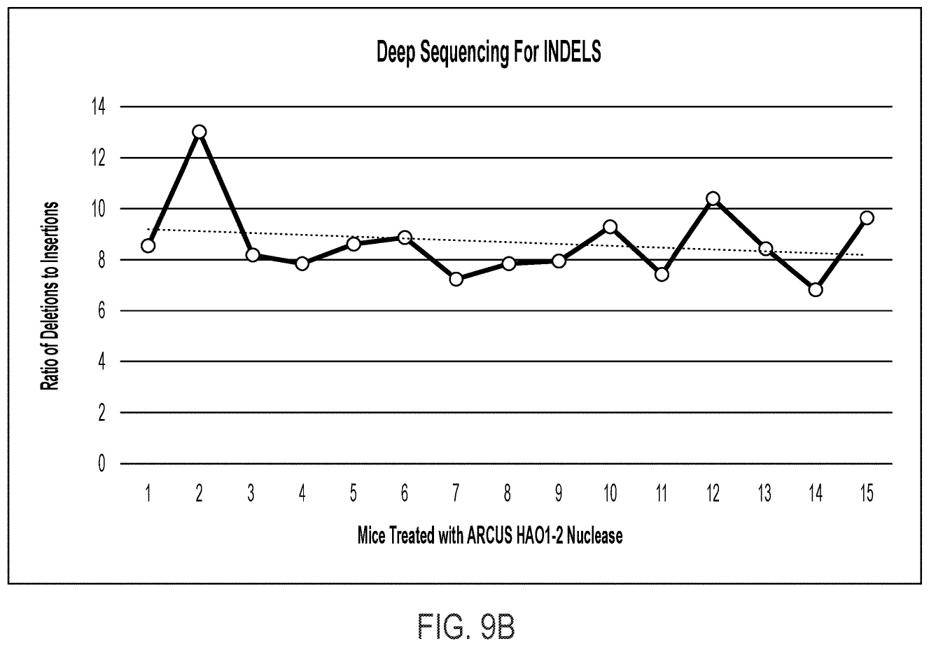

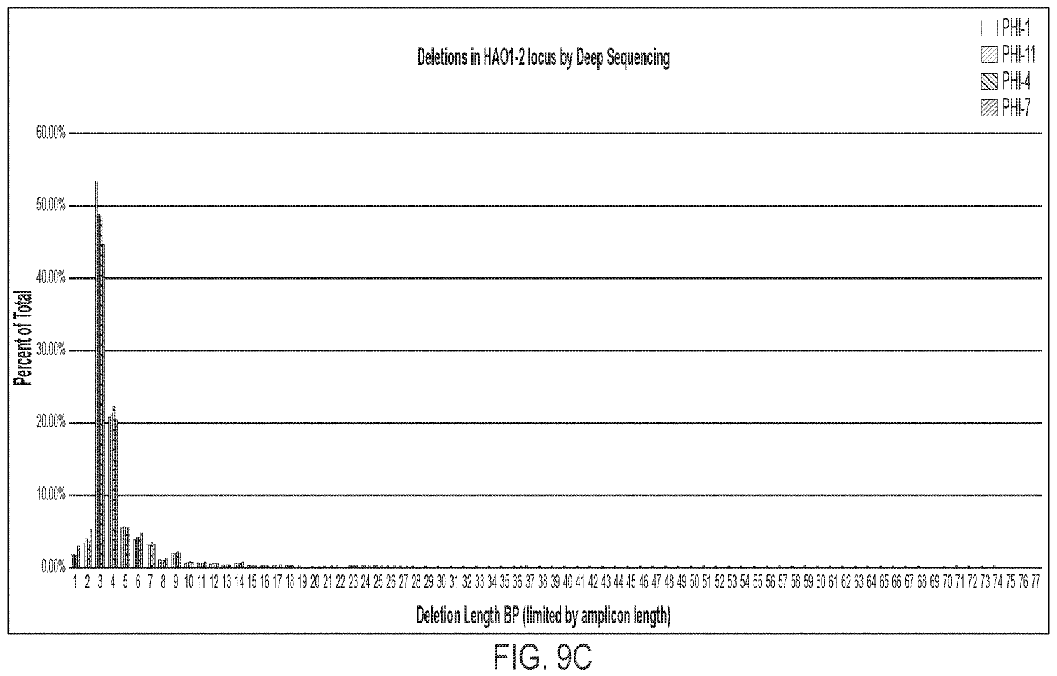

gDNA isolated from mouse livers were used as template in a digital droplet PCR drop off assay (FIG. 9A). A mouse reference probe was used to calculate % of edited HAO1. Indels were detected across all weeks and were greater than 49%. Treatment with HAO 1-2L.30 in mice showed consistent indel rates >60% at week 1 and are consistent out to 8 weeks. No editing was detected in mice that received PBS mock injections

The ratio of deletions to insertions was calculated by deep sequencing. Values were plotted and the slope of the line indicates that this ratio is constant across groups/weeks indicating that editing is not being selected out over time (FIG. 9B).

Deep sequence data was analyzed to determine the frequency of deletion, characterizing the most frequent size of deletions generated in HAO 1-2L.30 treated mice (FIG. 9C). Three bp deletions were found to be the most frequent with 50% of the sequence amplicons followed by 4 bp deletions at 20%.

Indels were analyzed by cloning and Sanger Sequencing to sample the frequency of deletions as well as determining the actual nucleotides deleted within the sample set. 41 sequences were analyzed of which 18 were the wildtype HAO1 sequence (44%), and 23 had deletions (56%). Of the deletions 10 (43%) had 3 bp deletions with Valine and Leucine deletions most prevalent. 1 sample had a 6 bp deletion and the remaining samples had 2, 3, 11, 13, and 26 bp deletions.

### 3. Conclusion

These results indicate that HAO 1-2L.30 is active in vivo and was successful in cutting the HAO1 gene at a high level. Treatment with HAO 1-2L.30 in mice resulted in editing of the HAO1 gene greater than 58% across all groups tested in vivo reaching the maximum editing at the earliest timepoint, week 1.

Deep sequencing analysis of each mouse showed no change in deletion to insertion ratio, which stayed constant across the different weeks indicating that there was no selection taking place.

Amplification and Sanger Sequencing of cloned PCR products around this binding site supports both the ddPCR and deep sequencing results with 56% of the sampled clones having indels at the HAO1 binding site.

### Example 5

### Mouse Pilot Study: Immunofluorescence of Mouse Liver

### 1. Methods

Tissue Prep and Staining

Mice were cleared of blood and organs were fixed by cardiac perfusion. Briefly, mice were deeply anesthetized using isoflurane and immobilized to a necropsy board. The thoracic cavity was opened to expose the heart. An incision was made in the right atrium and a butterfly needle attached to a 30 mL syringe filled with ice cold PBS was inserted into the left atrium. Slow steady pressure was used to perfuse the animal with 30 mL of PBS followed by 30 mL of Trumps Fixative. Liver was gently removed, placed in 5 mL Trumps fixative, and keep at 4° C. over night.

The liver was trimmed into individual lobes, quartered in a sagittal orientation, and placed in 30% sucrose/0.001% sodium azide over night to dehydrate. Trimmed and dehydrated sections of liver were imbedded in OCT in sagittal orientation for cryo-sectioning. Sagittal 5 μM sections were immobilized on glass microscope slides and were immediately cleared using graded ethanol, Xylenes, and Acetone. Marks were made around tissue with boundary pen and allowed to fully dry.

For staining, liver section were permeabilized using PBS 0.01% Triton then blocked with 2.5% normal goat serum, 0.001% sodium azide, and finally incubated with one of the following primary antibodies at 4° C. in a humidity chamber over night.

1. Abcam HAO1 Cat. No. 194790-1:100 in NGS

2. Abcam HAO1 Cat. No. 93137 1:100 in NGS

3. LS bio HAO1 Cat. No. C115788-100 1:100

4. Antibodies on line ABIN Cat. No. 2966702 1:100

5. Gentex—Cat. No. 84391 (human HAO1) used at 1:100 (negative control)

To tag the primary antibody, anti-rabbit Alexa-647 secondary Ab was used at a dilution of 1:1000, DAPI was used to label the nucleus at a dilution of 1:10,000, phalloidin-488 was used to label the actin cytoskeleton at a dilution of 1:50. Coverslips were mounted using prolong diamond and images were captured using a Zeiss Axio observer microscope.

### 2. Results

This in vivo study was designed to show the efficacy of the HAO 1-2 nuclease. Mice were IV injected with AAV expressing the HAO 1-2 nuclease which is designed to create a targeted indel to delete the peroxisomal targeting signal from the HAO1 protein. In the liver, HAO1 normally localizes to the peroxisome. Based on preliminary in vitro studies, is was expected that targeted deletion of the HAO1 peroxisomal targeting signal would prevent HAO1 from localizing to the peroxisome.

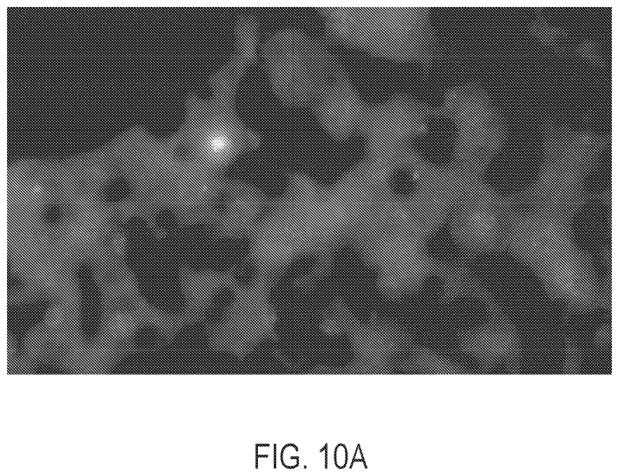

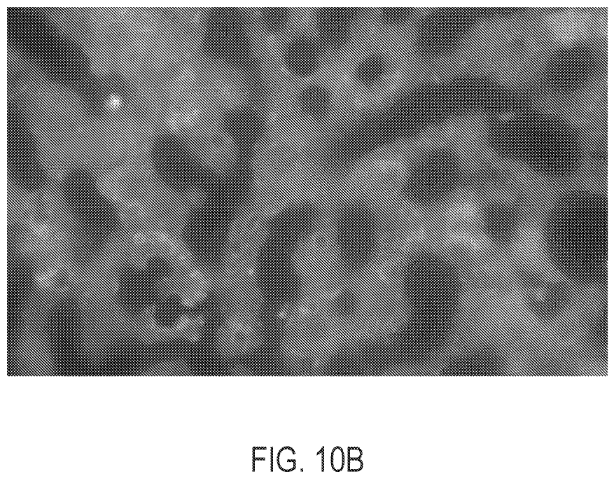

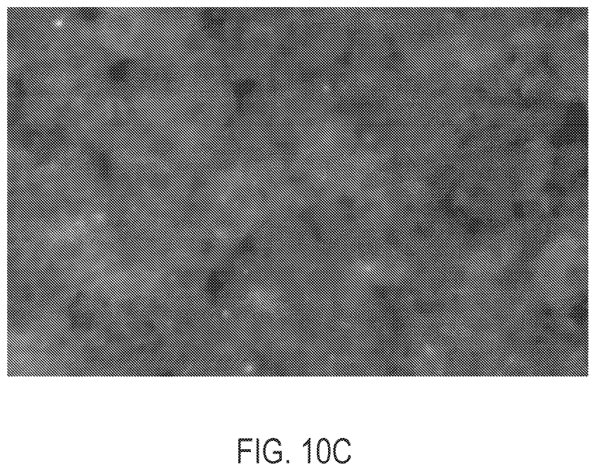

The data in FIGS. 10A-10C show liver sections stained by immunofluorescence for: nuclei in blue using DAPI; HAO1 in red using a primary+florescent secondary antibody (Alexa-647); and actin cytoskeleton in green using phalloidin-488.

FIG. 10A shows that the florescent secondary (Alexa-647) antibody does not stain control liver tissue in the absence of a HAO1-specific primary antibody. Staining of the untreated control liver (FIG. 10B) with an HAO1 specific primary antibody (Abcam HAO1 Cat. No. 194790) along with a florescent Alexa-647 secondary antibody results in the labeling of HAO1 (red) in discrete peroxisomal organelles. This untreated control animal in FIG. 10B demonstrates the normal wild-type localization of HAO1 in mouse hepatocytes.

FIG. 10C shows HAO1 staining in HAO 1-2 treated mouse liver with a HAO1 specific primary antibody (Abcam HAO1 Cat. No. 194790) along with a florescent Alexa-647 secondary antibody results in the labeling of HAO1 (red) in a diffuse pattern in a majority of cells. Relative to what is shown in FIG. 10B, this diffuse staining pattern is inconsistent with HAO1 localizing to discrete peroxisomal organelles. The diffuse staining in the HAO 1-2 treated mouse suggests that the HAO1 protein is mis-localized to the cytoplasm, which is consistent with the HAO1 protein not having a peroxisomal targeting signal.

### 3. Conclusions

The results of this study demonstrate the efficacy of the HAO 1-2 nuclease in targeting deletion of the HAO1 peroxisomal targeting signal and preventing HAO1 from localizing to the peroxisome in vivo.

### Example 6

### Mouse Pilot Study: Quantitation of Indels, Glycolate Levels, and Oxalate Levels in an AGXT Deficient Mouse Model

### 1. Methods

These experiments were initiated to determine if an engineered meganuclease could effectively target and generate indels at the HAO 1-2 recognition sequence in an AGXT deficient mouse model. In addition, this experiment was designed to determine if administration of an engineered HAO 1-2 meganuclease could affect AGXT deficient mouse urine glycolate and oxalate levels. Because the mouse model used in this study is deficient in the AGXT gene, these mice have basally higher levels of oxalate than wild type mice. Thus, this mouse model may more closely mimic the PH1 disease state in humans

Experimental Design

Cohorts of 3 AGXT-deficient mice received escalating doses of an AAV8 encoding the HAO 1-2L.30 meganuclease with a 3′ WPRE and driven by a TBG promoter administered by intravenous injection. Doses of the HAO 1-2L.30 AAV were 3e11, 3e12, or 3e13 GC/kg with a cohort receiving PBS as a control. The experimental and control groups are summarized in the table below.

TABLE 5

| Group | Vector Day 0 | Dose GC/kg | No. |
|---|---|---|---|
| 1 | PBS | N/A | 3 |
| 2 | AAV8.TBG.PI.HAO1- | 3e11 | 3 |
| | 2L.30.WPRE.bGH | | |
| 3 | AAV8.TBG.PI.HAO1- | 3e12 | 3 |
| | 2L.30.WPRE.bGH | | |
| 4 | AAV8.TBG.PI.HAO1- | 3e13 | 3 |
| | 2L.30.WPRE.bGH | | |

Murine Serum Levels of Urine Oxalate

Beginning on d0 before the first AAV8 injection, urine was collected at days 14, 28, 49, and 63 and levels of glycolate and oxalate were determined by LC/MS analysis.

In Vivo Indel % On-Target Analysis

Next generation sequencing (NGS) was used to determine on-target editing of HAO 1-2L.30 at the endogenous mouse HAO 1-2 target site. Using site specific primers, amplicons surrounding either the mouse HAO 1-2 target site were prepared and subjected to indel analysis by NGS.

### 2. Results

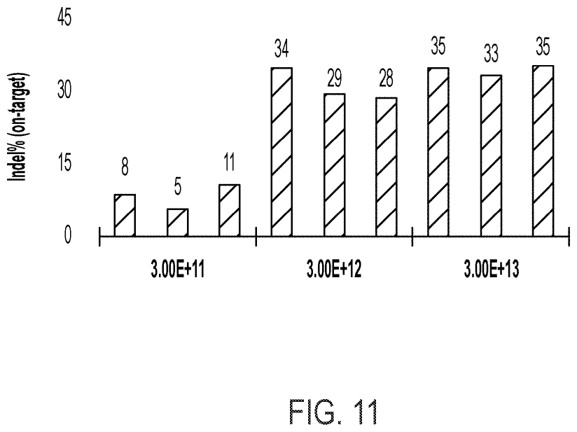

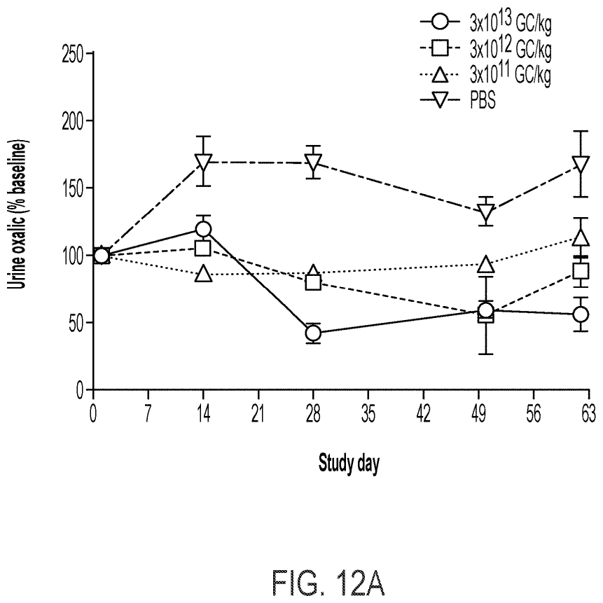

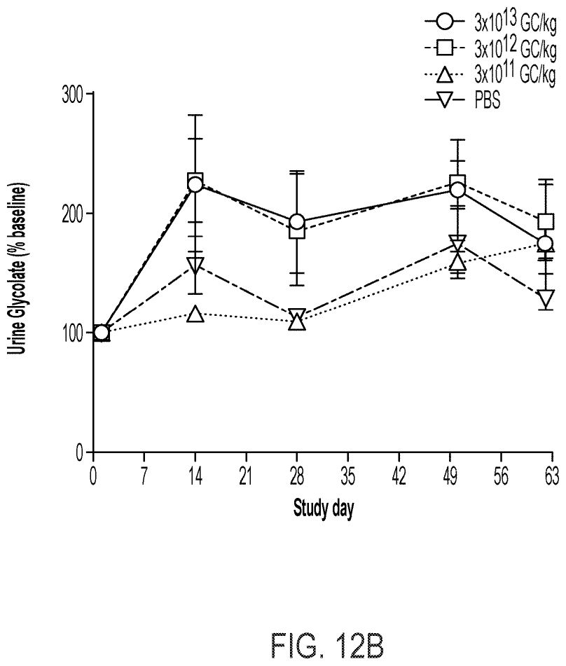

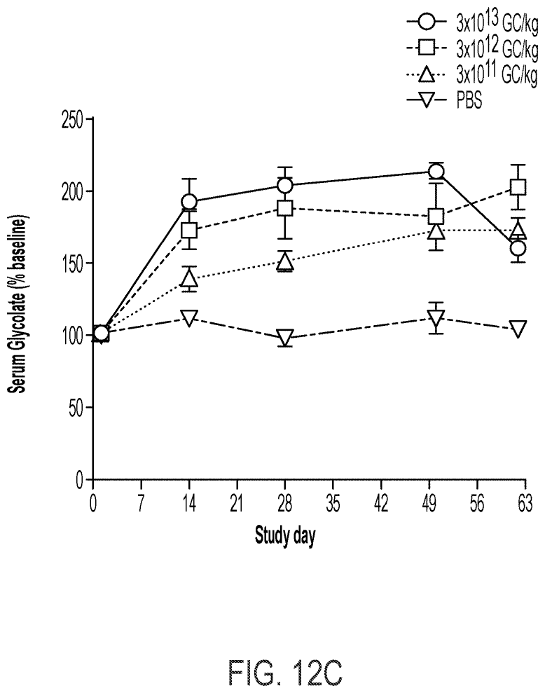

As shown in FIG. 11, the indel frequency in the mouse HAO1 gene showed a dose dependent indel frequency of 5% to 11% at 3e11 GC/kg, 28% to 34% at 3e12 GC/kg, and 33% to 35% at 3×13 GC/kg. Administration of the HAO 1-2L.30 meganuclease primarily resulted in deletions in the murine HAO1 gene. As provided in FIGS. 12A and 12B, mouse urine oxalate levels were decreased and glycolate levels were increased by administration of the HAO 1-2L.30 meganuclease. In addition, the mice showed an increase in serum glycolate levels (FIG. 12C). The reduction in oxalate levels and increase in glycolate levels occurred in a dose dependent fashion. Mice treated with 3e13 of the HAO 1-2L.30 meganuclease had the highest reduction in urine oxalate and increase in both urine and serum glycolate levels (FIGS. 12A, 12B, and 12C).

### 3. Conclusions

Data provided in FIGS. 11 and 12A-C demonstrate that an engineered meganuclease targeting the HAO 1-2 recognition site can successfully target and introduce high levels of indels within the endogenous murine HAO1 gene in an AGXT deficient mouse model. The editing was shown to occur in a dose dependent manner. In addition, administration of an engineered meganuclease targeting the HAO 1-2 site led to a decrease in urine oxalate levels and increase in glycolate levels in a dose dependent fashion. Thus, this experiment demonstrated that expression of an engineered meganuclease targeting the HAO 1-2 site, which is conserved between humans and mice, also had a significant effect on the biochemical pathway where HAO1 converts glycolate to glyoxylate in an AGXT deficient mouse model.

### Example 7

### Mouse Pilot Study: Quantitation of Indels and Glycolate Levels in Rag-1 Deficient Mouse Model

### 1. Methods

These experiments were initiated to determine if an engineered meganuclease could effectively target and generate indels in the human HAO 1-2 recognition sequence exogenously expressed in vivo in mice. In addition, this experiment was designed to determine if administration of an engineered HAO 1-2 meganuclease could affect mouse urine and serum glycolate levels.

Experimental Design

Rag1-deficient mice were administered 3e12 GC/kg of an AAV8 vector encoding the human HAO1 gene driven by a liver-specific TBG promoter on Day 0. Two weeks later (d14), cohorts of 5 mice received escalating doses of an AAV8 encoding the HAO 1-2L.30 meganuclease with a 3′ WPRE and driven by a TBG promoter. Doses of the HAO 1-2L.30 AAV were 3e10, 3e11, or 3e12 GC/kg with a cohort receiving PBS as a control. One additional cohort of 5 mice received PBS rather than the AAV8 hHAO1 vector, followed by 3e12 GC/kg of AAV8 HAO 1-2L.30 on d14. The Experimental and control groups are summarized in the table below.

TABLE 6

| | | Dose | | Dose | |
|---|---|---|---|---|---|
| Group | Vector Day 0 | GC/kg | Vector Day 14 | GC/kg | No. |
| 1 | AAV8.TBG.PI.hHAO1 | 3e12 | PBS | N/A | 5 |
| | native.bGH | | | | |
| 2 | AAV8.TBG.PI.hHAO1 | 3e12 | AAV8.TBG.PI.HAO1- | 3e12 | 5 |
| | native.bGH | | 2L.30.WPRE.bGH | | |
| 3 | AAV8.TBG.PI.hHAO1 | 3e12 | AAV8.TBG.PI.HAO1- | 3e11 | 5 |
| | native.bGH | | 2L.30.WPRE.bGH | | |
| 4 | AAV8.TBG.PI.hHAO1 | 3e12 | AAV8.TBG.PI.HAO1- | 3 × 10 | 5 |
| | native.bGH | | 2L.30.WPRE.bGH | | |
| 5 | PBS | N/A | AAV8.TBG.PI.HAO1- | 3 × 12 | 5 |
| | | | 2L.30.WPRE.bGH | | |

Murine Blood and Urine Levels of Glycolate

Beginning on d0 before the first AAV8 injection, blood and urine was collected and at every 14 days for the course of 8 weeks (d56). Serum was isolated from whole blood and both the serum and urine were analyzed for levels of glycolate by LC/MS.

In Vivo Indel % On-Target Analysis

Next generation sequencing (NGS) was used to determine on-target editing of the HAO 1-2L.30 meganuclease on the episomal AAV vector containing the human HAO 1-2 target site as well as the endogenous mouse HAO 1-2 target site. Using site specific primers, amplicons surrounding the human HAO 1-2 target site were prepared and subjected to indel analysis by NGS.

### 2. Results

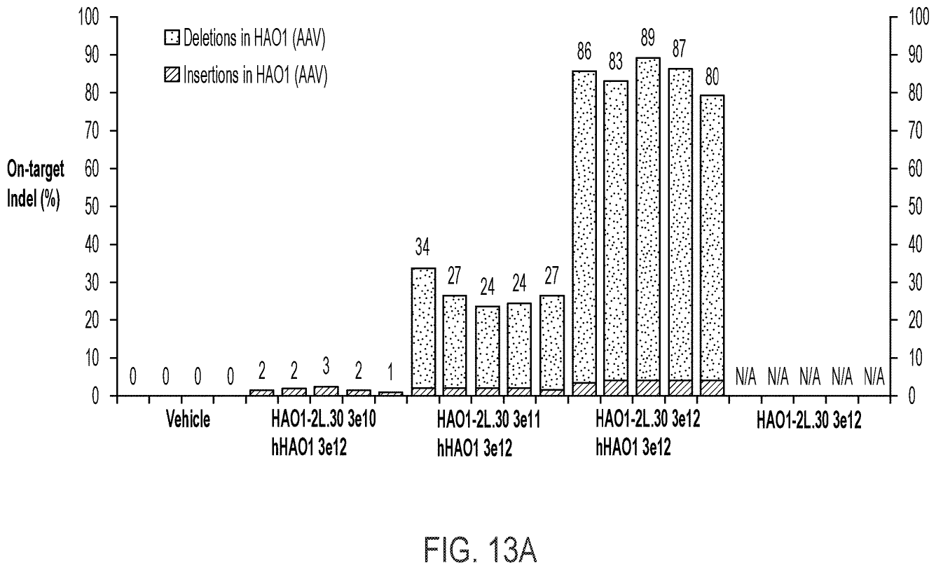

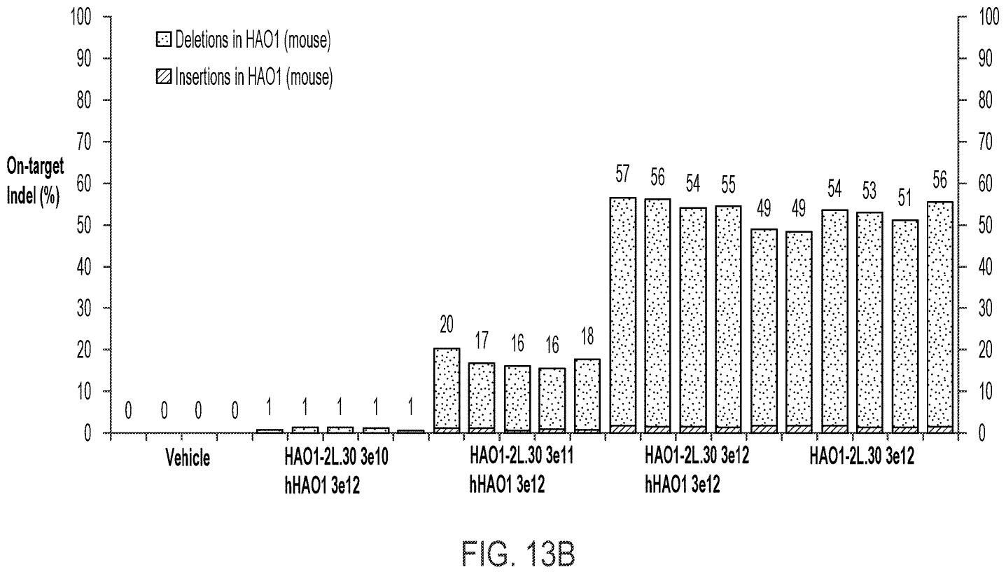

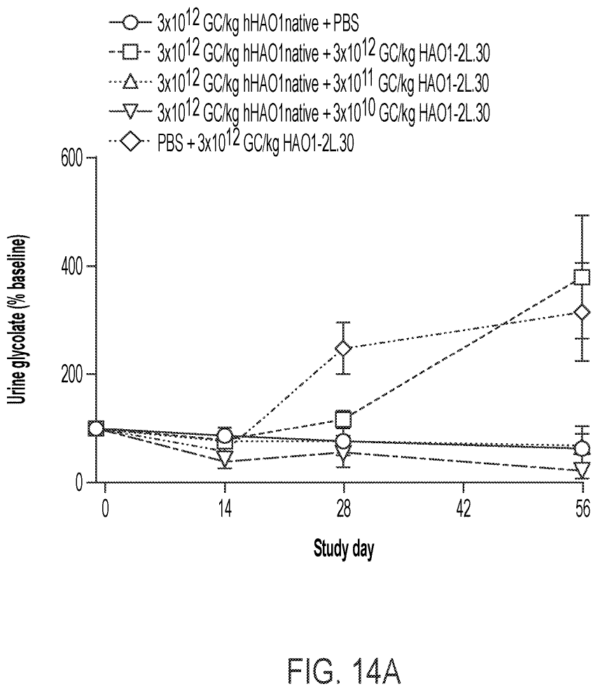

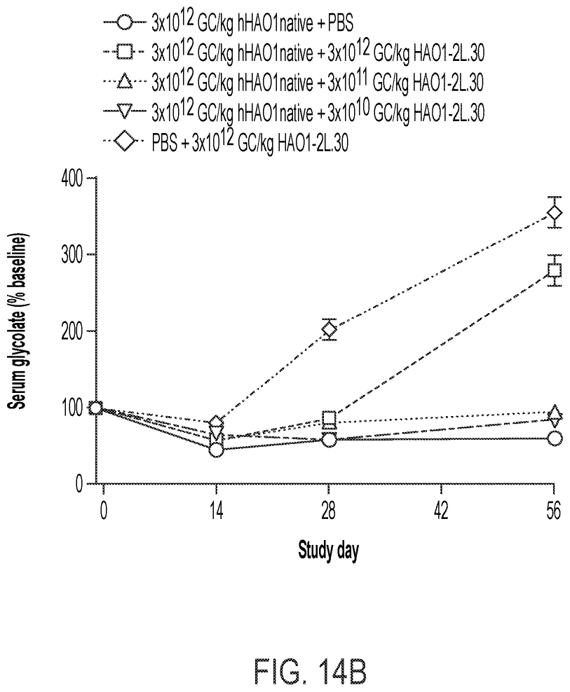

As shown in FIG. 13A, the indel frequency in the exogenously expressed human HAO1 gene showed a dose dependent indel frequency of 1% to 3% at 3e10 GC/kg, 24% to 34% at 3e11 GC/kg, and 80% to 89% at 3e12 GC/kg. Similarly, the indel frequency in the endogenous HAO1 gene in the mouse showed a dose dependent indel frequency of 1% at 3e10 GC/kg, 49% to 57% at 3e11 GC/kg, and 49% to 56% at 3e12 GC/kg (FIG. 13B). Administration of the HAO 1-2L.30 meganuclease primarily resulted in deletions with a small amount of insertions in the both the exogenously expressed human HAO1 and endogenous mouse HAO1 gene. In addition, both urine and serum glycolate levels were increased in mice treated with 3e12 GC/kg of the HAO 1-2L.30 meganuclease (FIGS. 14A and 14B).

### 3. Conclusions

Data provided in FIGS. 13A and 13B demonstrates that an engineered meganuclease targeting the HAO 1-2 recognition site can successfully target and introduce high levels indels within an exogenously expressed human HAO1 gene and the endogenous mouse HAO1 gene in vivo. The editing was shown to occur in a dose dependent manner. In addition, the data provided in FIGS. 14A and 14B show that the administration of an engineered meganuclease targeting the HAO 1-2 site led to an increase in serum glycolate levels in the mouse, which is consistent with the data of Examples 3 and 6. The reason for this observed effect of increased mouse glycolate levels is because the mouse HAO1 gene was also targeted by the HAO 1-2L.30 meganuclease despite the presence of additional human HAO1 gene, and the expression of murine HAO1 was likely reduced. This reduction in HAO1 gene expression levels would result in a concomitant increase in glycolate. Thus, consistent with data in Examples 3 and 6, this experiment demonstrated that expression of an engineered meganuclease targeting the HAO 1-2 site, which is conserved between humans and mice, had a significant effect on the biochemical pathway where HAO1 converts glycolate to glyoxylate.

### Example 8

Non-Human Primate Pilot Study: Quantitation of Indels in a Non-Human Primate Model

### 1. Methods

Next it was tested whether administration of an engineered meganuclease targeting the HAO 1-2 recognition site could generate indels in the endogenous HAO1 gene in non-human primates (NHP).

Experimental Design

Rhesus monkeys were administered either 6e12 GC/kg or 3e13 GC/kg of an AAV8 vector encoding the HAO 1-2L.30 meganuclease with a 3′ WPRE and driven by a TBG promoter. A liver ultrasound was performed on the animals prior to vector administration and at every 6 months throughout the study. From day of vector administration through weeks 8-12, all NHPs received prednisolone at a dose of 1 mg/kg/day orally. After 8-12 weeks following vector administration, animals were tapered off prednisolone by gradual reduction of daily dose. Liver biopsies were performed on day 18 and on day 128. From each liver biopsy, next generation sequencing was performed to determine in vivo indel %. In addition, RNA was collected for qRT-PCR analysis of meganuclease expression levels, HAO1 expression levels, and vector genome copies. Protein lysate was kept for further western blotting analysis of meganuclease expression. Histological analysis was conducted to stain for meganuclease expression and for inflammation using hematoxylin and eosin.

Blood was collected weekly through day 28 and biweekly for measurement of CBC levels in the serum, blood chemistry, and coagulation panels. In addition, monthly measurements were taken for immune responses in serum for antibodies to the AAV8 capsid and PBMCs. In addition, weekly measurements of oxalate and glycolate in the serum and urine was performed. Necropsy is planned to be performed at one year from initiation of the study for histopathological analysis.

In Vivo Indel % On-Target Analysis

At days 18 and 128 post-vector administration, a liver biopsy was taken according to the above described experimental protocol. The indel % at the target cut site within the HAO 1-2 recognition sequence was determined by amplicon sequencing analysis (AMP seq). In addition, the level of insertion of AAV inverted terminal repeats (ITR) was determined by AMP seq.

### 2. Results

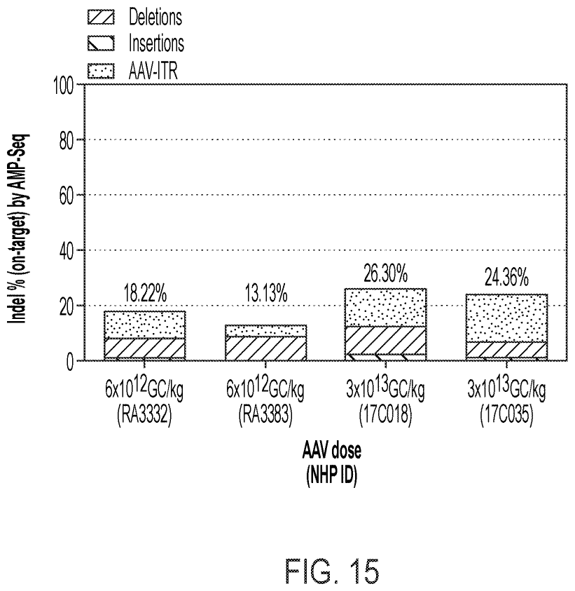

As shown in FIG. 15, administration of AAV encoding the HAO 1-2L.30 meganuclease resulted in a dose dependent increase in indel % in NHPs. At 6e12 GC/kg and 3e13 GC/kg of the HAO 1-2L.30 meganuclease an on target indel % of 13.13 and 18.22 and 24.36% and 26.30% was achieved, respectively. The indel % obtained at day 18 was maintained through 128 days post-administration (data not shown).

### 3. Conclusions

This study demonstrates that an engineered meganuclease targeting a site within the HAO 1-2 recognition sequence results in editing of the endogenous HAO1 gene within NHPs. The gene editing occurs in a dose dependent manner, which is consistent with the data observed in the mouse studies of Examples 4, 6, and 7.

1. An engineered meganuclease that binds and cleaves a recognition sequence comprising SEQ ID NO: 5 within an HAO1 gene, wherein said engineered meganuclease comprises a first subunit and a second subunit, wherein said first subunit binds to a first recognition half-site of said recognition sequence and comprises a first hypervariable (HVR1) region, and wherein said second subunit binds to a second recognition half-site of said recognition sequence and comprises a second hypervariable (HVR2) region.

2. The engineered meganuclease of claim 1, wherein said HVR1 region comprises an amino acid sequence having at least 80% sequence identity to an amino acid sequence corresponding to residues 24-79 of any one of SEQ ID NOs: 7-10.

3. The engineered meganuclease of claim 1 or claim 2, wherein said HVR1 region comprises one or more residues corresponding to residues 24, 26, 28, 30, 32, 33, 38, 40, 42, 44, 46, 68, 70, 75, and 77 of any one of SEQ ID NOs: 7-10.

4. The engineered meganuclease of any one of claims 1-3, wherein said HVR1 region comprises Y, R, K, or D at a residue corresponding to residue 66 of any one of SEQ ID NOs: 7-10.

5. The engineered meganuclease of any one of claims 1-4, wherein said HVR1 region comprises residues 24-79 of any one of SEQ ID NOs: 7-10.

6. The engineered meganuclease of any one of claims 1-5, wherein said HVR2 region comprises an amino acid sequence having at least 80% sequence identity to an amino acid sequence corresponding to residues 215-270 of any one of SEQ ID NOs: 7-10.

7. The engineered meganuclease of any one of claims 1-6 wherein said HVR2 region comprises one or more residues corresponding to residues 215, 217, 219, 221, 223, 224, 229, 231, 233, 235, 237, 259, 261, 266, and 268 of any one of SEQ ID NOs: 7-10.

8. The engineered meganuclease of any one of claims 1-7, wherein said HVR2 region comprises Y, R, K, or D at a residue corresponding to residue 257 of any one of SEQ ID NOs: 7-10.

9. The engineered meganuclease of any one of claims 1-8, wherein said HVR2 region comprises residues corresponding to residues 239 and 241 of SEQ ID NO: 9.

10. The engineered meganuclease of any one of claims 1-9, wherein said HVR2 region comprises residues corresponding to residues 239, 241, 262, 263, 264, and 265 of SEQ ID NO: 10.

11. The engineered meganuclease of any one of claims 1-10, wherein said HVR2 region comprises residues 215-270 of any one of SEQ ID NOs: 7-10.

12. The engineered meganuclease of any one of claims 1-11, wherein said first subunit comprises an amino acid sequence having at least 80% sequence identity to residues 7-153 of any one of SEQ ID NOs: 7-10, and wherein said second subunit comprises an amino acid sequence having at least 80% sequence identity to residues 198-344 of any one of SEQ ID NOs:
7-10.

13. The engineered meganuclease of any one of claims 1-12, wherein said first subunit comprises G, S, or A at a residue corresponding to residue 19 of any one of SEQ ID NOs: 7-10.

14. The engineered meganuclease of any one of claims 1-13, wherein said first subunit comprises E, Q, or K at a residue corresponding to residue 80 of any one of SEQ ID NOs: 7-10.

15. The engineered meganuclease of any one of claims 1-14, wherein said second subunit comprises G, S, or A at a residue corresponding to residue 210 of any one of SEQ ID NOs: 7-10.

16. The engineered meganuclease of any one of claims 1-15, wherein said second subunit comprises E, Q, or K at a residue corresponding to residue 271 of any one of SEQ ID NOs: 7-10.

17. The engineered meganuclease of any one of claims 1-16, wherein said first subunit comprises a residue corresponding to residue 80 of any one of SEQ ID NOs: 7-10.

18. The engineered meganuclease of any one of claims 1-17, wherein said second subunit comprises a residue corresponding to residue 271 of any one of SEQ ID NOs: 7-10.

19. The engineered meganuclease of any one of claims 1-18, wherein said second subunit comprises a residue corresponding to residue 330 of any one of SEQ ID NOs: 9 or 10.

20. The engineered meganuclease of any one of claims 1-19, wherein said engineered meganuclease is a single-chain meganuclease comprising a linker, wherein said linker covalently joins said first subunit and said second subunit.

21. The engineered meganuclease of any one of claims 1-20, wherein said engineered meganuclease comprises the amino acid sequence of any one of SEQ ID NOs: 7-10.

22. A polynucleotide comprising a nucleic acid sequence encoding said engineered meganuclease of any one of claims 1-21.

23. The polynucleotide of claim 22, wherein said polynucleotide is an mRNA.

24. A recombinant DNA construct comprising a nucleic acid sequence encoding said engineered meganuclease of any one of claims 1-21.

25. The recombinant DNA construct of claim 24, wherein said recombinant DNA construct encodes a viral vector comprising said nucleic acid sequence encoding said engineered meganuclease.

26. The recombinant DNA construct of claim 25, wherein said viral vector is an adenoviral vector, a lentiviral vector, a retroviral vector, or an adeno-associated viral (AAV) vector.

27. The recombinant DNA construct of claim 24 or claim 25, wherein said viral vector is a recombinant AAV vector.

28. A viral vector comprising a nucleic acid sequence encoding said engineered meganuclease of any one of claims 1-21.

29. The viral vector of claim 28, wherein said viral vector is an adenoviral vector, a lentiviral vector, a retroviral vector, or an AAV vector.

30. The viral vector of claim 28, wherein said viral vector is a recombinant AAV vector.

31. A method for producing a genetically-modified eukaryotic cell comprising an exogenous sequence of interest inserted into a chromosome of said eukaryotic cell, said method comprising introducing into a eukaryotic cell one or more nucleic acids including:
(a) a first nucleic acid encoding said engineered meganuclease of any one of claims 1-21, wherein said engineered meganuclease is expressed in said eukaryotic cell; and
(b) a second nucleic acid including said sequence of interest;
wherein said engineered meganuclease produces a cleavage site in said chromosome at a recognition sequence comprising SEQ ID NO: 5;
and wherein said sequence of interest is inserted into said chromosome at said cleavage site.

32. The method of claim 31, wherein said second nucleic acid further comprises sequences homologous to sequences flanking said cleavage site and said sequence of interest is inserted at said cleavage site by homologous recombination.

33. The method of claim 31 or claim 32, wherein said eukaryotic cell is a mammalian cell.

34. The method of claim 33, wherein said mammalian cell is a human cell.

35. The method of any one of claims 31-34, wherein said first nucleic acid is introduced into said eukaryotic cell by an mRNA or a viral vector.

36. The method of any one of claims 31-35, wherein said second nucleic acid is introduced into said eukaryotic cell by a viral vector.

37. A method for producing a genetically-modified eukaryotic cell comprising an exogenous sequence of interest inserted into a chromosome of said eukaryotic cell, said method comprising:
(a) introducing said engineered meganuclease of any one of claims 1-21 into a eukaryotic cell; and
(b) introducing a nucleic acid including said sequence of interest into said eukaryotic cell;
wherein said engineered meganuclease produces a cleavage site in said chromosome at a recognition sequence comprising SEQ ID NO: 5;
and wherein said sequence of interest is inserted into said chromosome at said cleavage site.

38. The method of claim 37, wherein said nucleic acid further comprises sequences homologous to sequences flanking said cleavage site and said sequence of interest is inserted at said cleavage site by homologous recombination.

39. The method of claim 37 or claim 38, wherein said eukaryotic cell is a mammalian cell.

40. The method of claim 39, wherein said mammalian cell is a human cell.

41. The method of any one of claims 37-40, wherein said nucleic acid is introduced into said eukaryotic cell by a viral vector.

42. A method for producing a genetically-modified eukaryotic cell by disrupting a target sequence in a chromosome of said eukaryotic cell, said method comprising:
introducing into a eukaryotic cell a nucleic acid encoding said engineered meganuclease of any one of claims 1-21, wherein said engineered meganuclease is expressed in said eukaryotic cell;
wherein said engineered meganuclease produces a cleavage site in said chromosome at a recognition sequence comprising SEQ ID NO: 5, and wherein said target sequence is disrupted by non-homologous end-joining at said cleavage site.

43. The method of claim 42, wherein said disruption produces a modified HAO1 gene which encodes a modified HAO1 polypeptide, wherein said modified HAO1 polypeptide comprises the amino acids encoded by exons 1-7 of the HAO1 gene but lacks a peroxisomal targeting signal.

44. The method of claim 42 or claim 43, wherein said disruption produces a modified HAO1 gene which encodes a modified HAO1 polypeptide having at least 80%, 90%, 95%, 98%, or 100% sequence identity to the nucleotide sequence of SEQ ID NO: 22.

45. The method of any one of claims 42-44, wherein said eukaryotic cell is a mammalian cell.

46. The method of claim 45, wherein said mammalian cell is a human cell.

47. The method of any one of claims 42-46, wherein said nucleic acid is introduced into said eukaryotic cell by an mRNA or a viral vector.

48. A method for producing a genetically-modified eukaryotic cell by disrupting a target sequence in a chromosome of said eukaryotic cell, said method comprising:
introducing into a eukaryotic cell said engineered meganuclease of any one of claims 1-21;
wherein said engineered meganuclease produces a cleavage site in said chromosome at a recognition sequence comprising SEQ ID NO: 5, and wherein said target sequence is disrupted by non-homologous end-joining at said cleavage site.

49. The method of claim 48, wherein said disruption produces a modified HAO1 gene which encodes a modified HAO1 polypeptide, wherein said modified HAO1 polypeptide comprises the amino acids encoded by exons 1-7 of the HAO1 gene but lacks a peroxisomal targeting signal.

50. The method of claim 48 or claim 49, wherein said disruption produces a modified HAO1 gene which encodes a modified HAO1 polypeptide having at least 80%, 90%, 95%, 98%, or 100% sequence identity to the nucleotide sequence of SEQ ID NO: 22.

51. The method of any one of claims 48-50, wherein said eukaryotic cell is a mammalian cell.

52. The method of claim 51, wherein said mammalian cell is a human cell.

53. The method of any one of claims 48-52, wherein said nucleic acid is introduced into said eukaryotic cell by an mRNA or a viral vector.

54. A genetically-modified eukaryotic cell prepared by the method of any one of claims 31-53.

55. A genetically-modified eukaryotic cell comprising a modified HAO1 gene, wherein said modified HAO1 gene encodes a modified HAO1 polypeptide which comprises the amino acids encoded by exons 1-7 of the HAO1 gene but lacks a peroxisomal targeting signal.

56. The genetically-modified eukaryotic cell of claim 54 or 55, wherein said modified HAO1 gene encodes a modified HAO1 polypeptide having at least 80%, 90%, 95%, 98%, or 100% sequence identity to the nucleotide sequence of SEQ ID NO: 22.

57. The genetically-modified eukaryotic cell of claim 55 or 56, wherein said modified HAO1 gene comprises a nucleic acid insertion or deletion within exon 8 which disrupts coding of said peroxisomal targeting signal.

58. The genetically-modified eukaryotic cell of claim 57, wherein said insertion or deletion is positioned only within exon 8, spans the junction of exon 8 and the 5′ upstream intron, or spans the junction of exon 8 and the 3′ downstream intron.

59. The genetically-modified eukaryotic cell of any one of claims 55-58, wherein said modified HAO1 polypeptide is not localized to the peroxisome.

60. The genetically-modified eukaryotic cell of any one of claims 57-59, wherein said insertion or deletion is positioned at an engineered nuclease cleavage site.

61. The genetically-modified eukaryotic cell of claim 60, wherein said engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

62. The genetically-modified eukaryotic cell of claim 60 or claim 61, wherein said engineered nuclease cleavage site is within an engineered meganuclease recognition sequence, a TALEN recognition sequence, a zinc finger nuclease recognition sequence, a CRISPR system nuclease recognition sequence, a compact TALEN recognition sequence, or a megaTAL recognition sequence.

63. The genetically-modified eukaryotic cell of any one of claims 60-62, wherein said engineered nuclease cleavage site is within an engineered meganuclease recognition sequence comprising any one of SEQ ID NOs: 5, 23, or 24.

64. The genetically-modified eukaryotic cell of claim 63, wherein said engineered meganuclease recognition sequence comprises SEQ ID NO: 5.

65. The genetically-modified eukaryotic cell of any one of claims 62-64, wherein said engineered nuclease cleavage site is a TALEN cleavage site within a TALEN spacer sequence comprising any one of SEQ ID NOs: 53-96.

66. The genetically-modified eukaryotic cell of any one of claims 62-64, wherein said engineered nuclease cleavage site is a zinc finger nuclease cleavage site within a zinc finger nuclease spacer sequence comprising any one of SEQ ID NOs: 25-52.

67. The genetically-modified eukaryotic cell of any one of claims 62-64, wherein said engineered nuclease cleavage site is within a CRISPR system nuclease recognition sequence comprising any one of SEQ ID NOs: 97-115.

68. The genetically-modified eukaryotic cell of any one of claims 54-67, wherein said eukaryotic cell is a mammalian cell.

69. The genetically-modified eukaryotic cell of claim 68, wherein said mammalian cell is a human cell.

70. A method for producing a genetically-modified eukaryotic cell comprising a modified HAO1 gene, said method comprising introducing into a eukaryotic cell:
(a) a nucleic acid encoding an engineered nuclease having specificity for a recognition sequence within an HAO1 gene, wherein said engineered nuclease is expressed in said eukaryotic cell; or
(b) said engineered nuclease having specificity for a recognition sequence within an HAO1 gene;
wherein said engineered nuclease produces a cleavage site within said recognition sequence and generates a modified HAO1 gene which encodes a modified HAO1 polypeptide, wherein said modified HAO1 polypeptide comprises the amino acids encoded by exons 1-7 of the HAO1 gene but lacks a peroxisomal targeting signal.

71. The method of claim 70, wherein said modified HAO1 gene encodes a modified HAO1 polypeptide having at least 80%, 90%, 95%, 98%, or 100% sequence identity to the nucleotide sequence of SEQ ID NO: 22.

72. The method of claim 70 or claim 71, wherein said engineered nuclease has specificity for a recognition sequence positioned within or adjacent to exon 8 of said HAO1 gene.

73. The method of any one of claims 70-72, wherein said modified HAO1 gene comprises an insertion or deletion within exon 8 which disrupts coding of said peroxisomal targeting signal.

74. The method of claim 73 wherein said insertion or deletion is positioned only within exon 8, spans the junction of exon 8 and the 5′ upstream intron, or spans the junction of exon 8 and the 3′ downstream intron.

75. The method of claim 73 or claim 74, wherein said insertion or deletion is introduced at said engineered nuclease cleavage site.

76. The method of claim 75, wherein said engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

77. The method of claim 75 or claim 76, wherein said engineered nuclease is an engineered meganuclease, a TALEN, a zinc finger nuclease, a CRISPR system nuclease, a compact TALEN, or a megaTAL.

78. The method of any one of claims 75-77, wherein said engineered nuclease is an engineered meganuclease having specificity for a recognition sequence comprising any one of SEQ ID NOs: 5, 23, or 24.

79. The method of claim 78, wherein said engineered meganuclease has specificity for a recognition sequence comprising SEQ ID NO: 5.

80. The method of claim 78, wherein said engineered meganuclease is said engineered meganuclease of any one of claims 1-21.

81. The method of any one of claims 75-77, wherein said engineered nuclease is a TALEN which generates said cleavage site within a TALEN spacer sequence comprising any one of SEQ ID NOs: 53-96.

82. The method of any one of claims 75-77, wherein said engineered nuclease is a zinc finger nuclease which generates said cleavage site within a zinc finger nuclease spacer sequence comprising any one of SEQ ID NOs: 25-52.

83. The method of any one of claims 75-77, wherein said engineered nuclease is a CRISPR system nuclease which generates said cleavage site within a CRISPR system nuclease recognition sequence comprising any one of SEQ ID NOs: 97-115.

84. The method of any one of claims 70-83, wherein said eukaryotic cell is a mammalian cell.

85. The method of claim 84, wherein said mammalian cell is a human cell.

86. The method of any one of claim 70 or 73-85, wherein said nucleic acid is introduced into said eukaryotic cell by an mRNA or a viral vector.

87. A pharmaceutical composition comprising a pharmaceutically-acceptable carrier and said engineered nuclease, or a nucleic acid encoding said engineered nuclease, of any one of claims 1-21.

88. A pharmaceutical composition comprising a pharmaceutically acceptable carrier and:
(a) a nucleic acid encoding an engineered nuclease having specificity for a recognition sequence within an HAO1 gene, wherein said engineered nuclease is expressed in a eukaryotic cell in vivo; or
(b) said engineered nuclease having specificity for a recognition sequence within an HAO1 gene;
wherein said engineered nuclease produces a cleavage site within said recognition sequence and generates a modified HAO1 gene which encodes a modified HAO1 polypeptide, wherein said modified HAO1 polypeptide comprises the amino acids encoded by exons 1-7 of the HAO1 gene but lacks a peroxisomal targeting signal.

89. The pharmaceutical composition of claim 88, wherein said modified HAO1 gene encodes a modified HAO1 polypeptide having at least 80%, 90%, 95%, 98%, or 100% sequence identity to the nucleotide sequence of SEQ ID NO: 22.

90. The pharmaceutical composition of claim 88 or claim 89, wherein said modified HAO1 gene comprises an insertion or deletion within exon 8 which disrupts coding of said peroxisomal targeting signal.

91. The pharmaceutical composition of claim 90, wherein said insertion or deletion is positioned only within exon 8, spans the junction of exon 8 and the 5′ upstream intron, or spans the junction of exon 8 and the 3′ downstream intron.

92. The pharmaceutical composition of claim 90 or claim 91, wherein said insertion or deletion is positioned at said engineered nuclease cleavage site.

93. The pharmaceutical composition of claim 92, wherein said engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

94. The pharmaceutical composition of claim 92 or claim 93, wherein said engineered nuclease is an engineered meganuclease, a TALEN, a zinc finger nuclease (ZFN), or CRISPR system nuclease, a compact TALEN, or a megaTAL.

95. The pharmaceutical composition of any one of claims 92-94, wherein said engineered nuclease is an engineered meganuclease having specificity for a recognition sequence comprising any one of SEQ ID NOs: 5, 23, or 24.

96. The pharmaceutical composition of claim 95, wherein said engineered meganuclease has specificity for a recognition sequence comprising SEQ ID NO: 5.

97. The pharmaceutical composition of claim 96, wherein said engineered meganuclease is said engineered meganuclease of any one of claims 1-21.

98. The pharmaceutical composition of any one of claims 92-94, wherein said engineered nuclease is a TALEN which generates said cleavage site within a TALEN spacer sequence comprising any one of SEQ ID NOs: 53-96.

99. The pharmaceutical composition of any one of claims 92-94, wherein said engineered nuclease is a zinc finger nuclease which generates said cleavage site within a zinc finger nuclease spacer sequence comprising any one of SEQ ID NOs: 25-52.

100. The pharmaceutical composition of any one of claims 92-94, wherein said engineered nuclease is a CRISPR system nuclease having specificity for a recognition sequence of any one of SEQ ID NOs: 97-115.

101. The pharmaceutical composition of any one of claims 88-100, wherein said eukaryotic cell is a mammalian cell.

102. The pharmaceutical composition of claim 101, wherein said mammalian cell is a human cell.

103. The pharmaceutical composition of any one of claims 87-102, wherein said nucleic acid is an mRNA.

104. The pharmaceutical composition of claim 103, wherein said mRNA is encapsulated in a lipid nanoparticle.

105. The pharmaceutical composition of any one of claims 87-102, wherein said pharmaceutical composition comprises a recombinant DNA construct comprising said nucleic acid.

106. The pharmaceutical composition of any one of claims 87-102, wherein said pharmaceutical composition comprises a viral vector comprising said nucleic acid.

107. The pharmaceutical composition of claim 106, wherein said viral vector is a recombinant AAV vector.

108. A method for treating primary hyperoxaluria type I (PH1) in a subject in need thereof, said method comprising delivering to a target cell in said subject a nucleic acid encoding an engineered nuclease having specificity for a recognition sequence within an HAO1 gene, wherein said engineered nuclease is expressed in said target cell, wherein said engineered nuclease produces a cleavage site within said recognition sequence and generates a modified HAO1 gene which encodes a modified HAO1 polypeptide, wherein said modified HAO1 polypeptide comprises the amino acids encoded by exons 1-7 of the HAO1 gene but lacks a peroxisomal targeting signal.

109. The method of claim 108, wherein said method comprises administering to said subject a therapeutically-effective amount of said pharmaceutical composition of any one of claims 87-107.

110. The method of claim 109, wherein said modified HAO1 gene encodes a modified HAO1 polypeptide having at least 80%, 90%, 95%, 98%, or 100% sequence identity to the nucleotide sequence of SEQ ID NO: 22.

111. The method of claim 108 or claim 109, wherein said engineered nuclease has specificity for a recognition sequence positioned within or adjacent to exon 8 of said HAO1 gene.

112. The method of any one of claims 108-111, wherein said modified HAO1 gene comprises an insertion or deletion within exon 8 which disrupts coding of said peroxisomal targeting signal.

113. The method of claim 112, wherein said insertion or deletion is positioned only within exon 8, spans the junction of exon 8 and the 5′ upstream intron, or spans the junction of exon 8 and the 3′ downstream intron.

114. The method of any one of claims 108-113, wherein said modified HAO1 polypeptide is not localized to the peroxisome.

115. The method of any one of claims 112-114, wherein said insertion or deletion is introduced at said engineered nuclease cleavage site.

116. The method of claim 115, wherein said engineered nuclease cleavage site is within exon 8, within the 5′ upstream intron adjacent to exon 8, within the 3′ downstream intron adjacent to exon 8, at the junction between exon 8 and the 5′ upstream intron, or at the junction between exon 8 and the 3′ downstream intron.

117. The method of claim 115 or claim 116, wherein said engineered nuclease is an engineered meganuclease, a TALEN, a zinc finger nuclease (ZFN), a CRISPR system nuclease, a compact TALEN, or a megaTAL.

118. The method of any one of claims 115-117, wherein said engineered nuclease is an engineered meganuclease having specificity for a recognition sequence comprising any one of SEQ ID NOs: 5, 23, or 24.

119. The method of claim 118, wherein said engineered meganuclease has specificity for a recognition sequence comprising SEQ ID NO: 5.

120. The method of claim 119, wherein said engineered meganuclease is said engineered meganuclease of any one of claims 1-21.

121. The method of any one of claims 115-117, wherein said engineered nuclease is a TALEN which generates said cleavage site within a TALEN spacer sequence comprising any one of SEQ ID NOs: 53-96.

122. The method of any one of claims 115-117, wherein said engineered nuclease is a zinc finger nuclease which generates said cleavage site within a zinc finger nuclease spacer sequence comprising any one of SEQ ID NOs: 25-52.

123. The method of any one of claims 115-117, wherein said engineered nuclease is a CRISPR system nuclease having specificity for a recognition sequence comprising any one of SEQ ID NOs: 97-115.

124. The method of any one of claims 108-123, wherein said nucleic acid is an mRNA.

125. The method of claim 124, wherein said mRNA is encapsulated within lipid nanoparticles.

126. The method of any one of claims 108-123, wherein said nucleic acid is delivered to said target cell using a viral vector comprising said nucleic acid.

127. The method of claim 126, wherein said viral vector is a recombinant AAV vector.

128. The method of any one of claims 108-127, wherein said subject is a human.

129. A recombinant HAO1 polypeptide comprising the amino acids encoded by exons 1-7 of said HAO1 gene but lacking a functional peroxisomal targeting signal.

130. The recombinant HAO1 polypeptide of claim 129, wherein said polypeptide is encoded by exons 1-7 and at least 3 bp of exon 8 (SEQ ID NO: 4) but lacks a functional peroxisomal targeting signal.

131. The recombinant HAO1 polypeptide of claim 130, wherein said polypeptide is encoded by exons 1-7 and 3 bp-62 bp of exon 8 (SEQ ID NO: 4) but lacks a functional peroxisomal targeting signal.

132. The engineered meganuclease of any one of claims 1-21, for use as a medicament.

133. The engineered meganuclease for use according to claim 132, wherein said medicament is useful for treating a disease in a subject in need thereof, such as a subject having PH1.

134. The engineered meganuclease of any one of claims 1-21, for use in manufacturing a medicament for reducing serum oxalate levels, reducing urinary oxalate levels, increasing the glycolate/creatinine ratio, decreasing the oxalate/creatinine ratio decreasing the level of calcium precipitates in a kidney of the subject, and/or decreasing the risk of renal failure in a subject, such as a subject with PH1, or a subject with increased serum oxalate levels.


SEQUENCE LISTING (128 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 57463 bp, Homo sapiens
ctgggatagcaataacctgtgaaaatgctcccccggctaatttgtatcaatgattatgaacaacatgctaaatcagtacttccaaagtctatatatgactattacaggtctggggcaaatgatgaagaaactttggctgataatattgcagcattttccaggtaagaaaatttattttttaaaatcatgttttaaaattacacaaagaccgtaccaaaataagatctcctagttttacgttggtggtgtgtaattatttgttcagatttgtgcttagtagagagggaaaagttcttggggctgtaagaaatcttgggcctttaaattgttaaaaaatattccaagcctgtgaatcttgaggaactgactgcaaaagccaaacctatgttacttcacttggaaatatgacaacaattaatttaactacatgtaaaaatagcgataaattcggatgacttttctttttcttagtatgacagtaaatgcttatgttcatggtgtaggaaacagcattaaatgccagataaccatcttatccggatgaaccagactggattgttggctcaaatgttttcttcctgctggcttttcgtgttatcattcattttgattactgttgtctaaactttcactttagatttcaatttgtctatgcagcattaatctttcaactttgctgtttcatctctccttcaaagcacttcatctctcttcccaaattagttttcctttgactttcatatttcaaagcacaagatggtgggtgacatggtttatgttttctgtttgtaataaaaacaagaaataaaatcatttcaaagggtttttttttatagcagttacaaaaatggtttatttgctggagcaagagaggagtgccttcactacactacactcagtctcatccatctaacattatggctgttagtaaaggcaatcggtattgtgggtactcattgatggtgataaagacaaaaaggcagaaaatatgcagggggagagaattagccttcctccctgatttcttctttagtctacaacaaaatcactcaaaatcagttttccatatttaaattaggagaaataaaattatcctggccaaggtggtctctggtaggcagcactgattcacccacaaatccatgtagaagactgaaaatggcaatggggtgaaggatacggcctctccccaaccctttcaagccttgactttgtctcaggttttgcctggaacccaaatgagctcaacaaatgccagggaagtcatgggaagggaagttgactgagagtagaggggcttaaaattctgcatcattatttactattttggactcatttaaaagtttctgctcttggaagatgccccttcttgggccgatattaactttgtccaccaaaatttgcctatgagtggtctcttgaaaacactttaacccaaataggttattacaaccaaggaaatttcagacccttgacagatttatagagttagtgtctcagcattgctagacctccaatgctcaagtgattatttatttcatttgtatacagctttccttacttcttaattccctttgtcgcatgctagctaattaactagagctaattaggagtctccatgagctacactgtgtactacatgctgaggacaaagcagtgagccagacaaagttcctgtccctaggaacttacattcccctggatgcatatcagcctccataatgctgttgggttgaattgatgcaaaatgggcccaaaatagttggccaagtggaggtctcagagaggatgcaaaggggcgccccaaagcagatggatcacctatgcaaccctttaaaatgtagaaactttgggagacatagaaggcttggtgacttctaagttatgaactggaaaagtgcctcatgccttatgtgaattacatggtattcaagtgagtattcccatcctatgtgtgtaccgagtaacttagggataggacacagataatgaaaatgaatttgcagtgtcaccttttccatgaaccttgatcattctcttttgttcagctttaaattaaaaaaaaaaatcaatcaactttctttggaggacagctgatgctattttattatcaactagttgagtttttattgcaatacattttgcaatgtgtcctcttttgctgtatgactcgctaggtgaaccttgattcctcacactgcatcatgtagctggtcacgtgaaactaagaatagaaattctgccagggttgtggagactttgggttgatggcatgaaggaaatcaacctgaaatttcacattctgattctaatgaaaagtgcaaaacaatcaaacctcagataacccattgtgatacaaagccagagtatttcaaacacatttatgaaatttatacacctccccatctcgcaagtacaacaaaaggtcattcaccgtgacagcttttatttctctgtactcagctctgataatcacattttggagttctggggacatggaccactcatgtgacccagcagttgcttggagatatttttgggtaagacttcagactaatattactgtggcagtagaaaaaaatgtttaaaaggacaagtaaatggaaccacccagaacaaaatttcttacggtggttataacaaaacagggtaaatgtcaacttgctacattttgcatggctggaattgattgggattaattcaacgaagaacagtaatttgtttctcttacacatttattcaaagtagccttctcaactatggtcttcacgttgttgtagcttttttttctgaaattatcaatgatggaagatgattaaacaatttcgacacttagaagccctcatgatttcagaaaaggaaactcttttctgctgcgttacctattgagactgaagatggcatcattttcttttaaataacagatgggtaaaagtgatgtcattctttcactttaatatttgagaagtgatatgaagttaccagtgacattgtgttctcataggcataaatgtcacaaaataatttatctagtatccacaataggtgaataaggtgtttttgctttatatattttaactgtttagagtaaaaaattaatgtggagaaaattggaatgcagtattataggattacacaacttacaaaacatgaatccactatgtccagttagtgtgattcagaaacagcatgcagttataaagctgggtgaggcatgggtgtcttccttcaacagggcagctactttgtgaggagtgtatatatcatttgatttttttataagttaaatttgaggcccctgttagatgtgagggtgggccaaaattcctgtgaacagattctccccgttaccccgcttcctttactctggcatctcattttctatcctttgaaaacggtttattattcaattggttcaactgtttgccagttgaaccaattctttttccaaagtggaggcccaggaaagcacagtccgagaatatagtgaggtgctattttatgtatgattgtgggaaatttacttaaatttggagtggggttgggcaaggcttggaaagctagtgagctatctgacatagttgttactactatttgaaaaatatcaaaacatggaggactctttagataacatgcctgttcccattccattgattttatctaattttacgtagcaattacgttttgtgcattggttgacaagcctctgtattatcctcagaacagaaaatactgtttaagggaaattaagagcccgcagttactaaagtgactgcgccaccaagtggacaagtgtaaagccactgtctggagatggaaggattcagctttgctttataaatgggaatttgacctttaaaaatgtcccttttggcacgcacgcgcgcgcgcgcgcgcgaacacacacacacacacacacacacacacacacacacacacacacggctgctgccctgcagatttgcttgttcttgtcataaagctttcattgtttctctagctctaagtaaatattaatgccttccaaggctggcatgccaatggctgctattaagatcgttttctctcattctaataacacacttagagatgattggtaataaaaactctcttcaaggcttctgcttctcccccttcaaaatggagatcaaagaatcatgctgtgagggtccgtcaagaagaaaagactttcagcaacagagcatgtggtgtggcataaaataatgacaattataatgttcaaaggaatagcatagaaatcacacagtaaaacttctttattatgcttttcagggactggatgtttttactttattatgtgaggaagggttagattacagacccttagctattccacaaagcaatagaaggcagaatttcttcttccgctacaggaagcacgcttcgattaagggctttttctttttcttctttttttttctttaagttactgcattactatatcatacttcactatatttactaaaaagtcatgctgtttctggaagtagagttacatctaggaaatactaggtgaatgctggttagatatgcatgtgtgcctaaacaacacgtttattatactcatgcatactagaaatagggctgtattttcttcaattttaatcagtactaatgagaataataaatcaaaacaaataggagagatatattttgccaggaggaaagagaactagttcttctgtaaattttactggtgaatttttggttgctggtttattggtaattttcattccaacacagaagaatcacagaaacattcatttaaaataattttccggagtcaaaaactttttaacacccaaatttcagtttttgtcaaataacatttttgagaaaagtgttaaattaaactaataaaaaaccttccctcatcattagactttaatgaatatggcatataactaaataattttgaagaaaccaaattataattttaaaagtaattgcctgaagctgctgtttatcacataaaaagaagacaaactagacatagcatatcttcttaaactctaatctaaactctatgcatttgtataccatcttgattttcaagattggggaagtgaaacgaaaactatgttcacacaagaacctgtacgtgaatgtttgtagtggctttatttagaatttccccccaaactgtaagtattcaaaatgtcttttagcttgggaatgactggacaaatgatagtacccctgtatgatggaatattattcatcaaccaaaaggaacaaactattgacacgtacaacaacatgagaaaatctctaatgcgttatgttaagtgaaagaagccaaactcaaaaggctacatactgaatgattttgtttacatgatattcttgcaaagcaaaattatcaggacaaagaaaaaatgcatcagtggttgtcaggggattgaactggggagagtttctctgcaaaagaaaatggggacttttttgggaatgattgaactttttctagatcttgattgtcatggcagttacaccactgtatgcatttgtcaaaattcacaaaactgcagactaaaatgagtgaatactattatgtattagttatactttaataaataattgcttgggaaattcattatcctctaattgttaactttctaaccaaacaaacagtaaaattgcctcttttccattagctttatgaagtcatttgcttgtttggaaaaaatccaattatattttttcttttaactaaaatgtaatgtcaaagttttggttatgattctgaaactctaaagccttttattttattttattttttaattctagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagaggtaggaggaagattgtcaccacagggacagaaggaggctaacgtttatcgacctccttctctgaatgcaccaagcaaatatgttccttgatgtttttacactcagaaacattaagctcatggactctatcatcaaaatacttgttcttgcatgtcctgctcctcttctttccagctgtgtgactgggcaagatatcctctctctgcattggtttccttggctgtaaaatagggacaaaaattgtacctgcctcattgggttatggtgagaattgaatgagttcaggtatacaaagttcatggcagagagtaggggctcagtaactgttggttatattatgggtattaatagtactgtctcaggaaatggatctctgacaggtagacttgcccaaagtcacagctaggtagttacagaattggaattcagccctgtggctaccttatctcaaaaccctcctgcttcccccaaaccaaagtggttctcacagccaaattgcaaatggagcaacgtggttggttgtgttttcttccgtggttttgggtcatgattcttttttatggatgagttatattcccaatagagcagttccagctgtcttaggagggagtgatgagaaaatcaaatatgatgtaaagaaatctcttattagggctaatttattaactttccagttctctagcaactgtgaacatttgaaaggctgtgcagagtaaaaaatctccccaaattgtgctccagaaactaatataaaagttggaaatgaattattttgatgctaagcagagcagaaaaagaacacgactatataatattttaaaacattttagttttaagaattaaggatcttgtgaattcacttcccttcttgaaatgtctgacataaaattctgtcagggatatcagaatggcacaatgaggttttgctggacagacttagcagcttccttaattctaggaccacatacaaataagtggctttggggcctcagccttttgtctatggtaatcctgaaacataagtagagagaagaaaaaaaaagggaaatactaaatgggtaaatatctatacaaaatcaagataataaaggccctttcaggcttgaaactataggcaacaaccttagaacaaaagaaaacaaatgaacatcaaaaaactaaaactttagtgctcttaaatctcaatgaaaataaaaagtaaatggtaaactgaaagaaatggaaaaaaaatatgagactgtgaagggttaatgtcctttccacgtaaaaagcccttatatttgaagaagaaaataatatattgctcaaagggaaaaagagaaataagtgaacaaaagatataaataggaaatttacaaatggagacataaaagtgaccaataaacatatgaaaaatattcaatttcattaataagcaaagacatggaaattatgaccatctattttattttccgtatatcgaatttttattttaagatcaggcagtatgattaggttagggagaaaatgtgcatttcaaacagtgttgagaaaagtataaagtggaataatcttcctagaaaataatctggcactgtatatcaaagctctaaaaatgtaaattccatgtgatgttaaaaattctcttctaggaattccaaggaaataattatgatttttgaggaaaaaaatcatttctgcaaggattttcatgcttcttatttttagcaggaaaataatttgaaaaaaatacccaaacatcttataattggagatagtttgcaaaaaatatgatgcataaaaatgacatcaaatttaaaaattatactataggaagagtgcaataatgtagaatgatattttaatttaaaattgtgagaaatcagttgcaaacaatagtcaggtcctaaaatacatttagtttcaaagatcacaatttacaaatgtttatttataagtgatgagattactcctgactttatactcttctgatttttggctcaaccttataaactcttctttgaattattttgtaaggaggaaatgataacaattagatttaaaagagtagagataaagggacaagggaccatgaagagaatggaaataaagaaaggaagcagagaaagcaaaaagcagagctcacttggtaaggcaccctggagccagcaaattatttttaccacatgtattagttccttctcacactgatttaaagatactcttcgagactgggtaatttattaaggaaagaggtttaactgactcacagttctacatggctggggaggcctcaggaaacttacaatcatggtggaaggcaaaggggaaggaacgaccttcttcccatggtggcaggagagagaagtgcaagcagggaaatgccagatacttataaaaccatctgatctcatgagaactcacccactatcatgagaacagcatgggggaaaccacccccatgatccaatcacctcccactaggtctttccctcaacacctgggattataattcaagatgagatttggatggagaaacaaagcctaaccataccaacacatattgctttatttgatatttgacaggtgtttctgtccctgttttgtgggcaagtagctaaagttccagagaaaacagtttttcatagctcgtcaatgacagacttattctccaagtcacatttgatggttccaagaccagtctttattcttggtggagttgggctgagaagaaagaggagaagaaagaagaaaagaaagcttccttagaaactatgatttgacagtgtaagtaggactatttcctccagaagtaaccataagaagatattaaatgcctattacagtcttatccccttagatttatttaacacttataaagcaattatcatgttccagacactattttaagtatattacgagtattatagcattgaaggctcagagcggcccaaataaatcgatcatattattaaacctattttacacaggagaaactgaggtacacgccaggtgaataaccttgcctagggatgcacaattcataagtgatagagatgggattcagacagaggtattctgtctccagaatctgggctcctcaccactttgcaagagctttaatttcagaaactcctatgaagtgtcatgaggagaagcccattatgatcccctagaagtaattatagttttaggagcatgcaaagcagacccctcaggaagataagttacacaatagacatttggataaggtggatccagcagaacaaagagagggtggtgacatcgagattgcagaggaattggagaaggcaatggaagtgtacacatgttgccctcaaaaacatagggtcctccattgggttcctatcagggcagcaacatcagagtttctattctgtatttatactagaaacctctctccagggtttctaagttttcacctatgttttaaagactatctataggttattagtctatttaatatttaggtgtatccagaaagctgatggtcatcagctcatagcaggtgttctttggctggtgtgtttatgttgtgggacagtgggttacttgcaaggaaaggatgaatggctggagtagatggtgcttgtgctctgcatgtattcccttcttacttcccatttccatcagacctaccactttttgcctgacattatctgttgcaacatgagcccatggataggtgtgtttgaagtaggggaatgggagagagggttccctagctaatgatgtacagcagtaggtggataaatacctcagctctctttgctcaggtaactgaagcattttctaatatggtcacccagtgttccttggaaggattgagtcccagttgccccctgaggttgcctgcccatgaacacaccctcttttattggcttccttcccattcttttctcacttccccattccttcaattcattgagattgtttccaaataagatgacttgctctcacatctctgtgtcatttttggcttcttgaagtatgcaaaccaggataatagctaactgaaggctatagatagccacaggcaaatttaagtaacagtgtaagaatattcatacttggcagagatttatttataaaaactcagaaaattcacatggaattatgaagttattattgtatttattccatcattcccagaaagaatatggaaatcctctcaagcaagccagtccttgggaatattgggaaatctatgcaatttgttgtggagtatttttttttttgttaccctcctaaatatctggccgctaagcattcctgtctccagggacttagaccctagcaaggaagagaagttggggccaggttcagaaaacgggttagttatcaatctccctggagaagtgtccccctcagcagggtcagtgagagtaagtgaaacccattggtgcccacaggcaatggtctggcctgagtaattagaatgggcctccagaaagttctgggaattgctatggtgccatagtctcattttccccgttgactctccagatttattcagagtccaacttcaagggcctttctgcccttcctctcacaactgtggaataataataatccaccttattaactgggaccgagaactgagctcgactcttatttttttgagacagagtcttgctctgtcaccagactggagtgcagtggcactatctcagctcactgcaacctctgcctcccaggttcaagcgattcccctgcctcagcctcctgggtagctaggactataggcacgcaccgcgacggctggctaattttttgtattttagtatagacagggtttcaccatgttggccaggatggtcttgatctcctgacctcatgatctgcctgccttggcctcccaaagtgctgggattacatgcgtgagccaccgcgccctgtctgaactctacttttttacactgctgcatgtttgtagagtgaccaatgaagctatacttttttcattttcaaaatgatgatgaatacaaggttatcaaataaaacacagagggcccattatgtttgaatttcagataaacaacaaatcataggtgtcctgtatgtttgctcaatctggcaaccctggatgaataagagctctcacctgaggatttcttgtgaggattcatgaaataaatgctagaaatgcttacacactatctttatttgcccctcagagcccaaagtctctgaaatctttatctttcacacacaaaaactcactttcagaaaagtatattccatttacatctagtggaaataaaaattgttctttttctttgtgaaaaatatttttattttaagctttatgcagaaacctcagggaaaaaaaggtacttttaggagccaggcttgtaatgtaaatgtccaaaaaagatgaaattgaaacaaacaaacaaacaaacaaacaaacaaacaaacaaaaaacagtgcaagctcctgtgtggagactgcagtgagtctgagattgcatgttccatcagaagggggcagccacatcttagctcttgatgacccaagggagcagggatgtggggttgccaaatcttccaaaattttaagaagccagaaatcttgatttctatgtacaatctcctggtttttaaatgtgggcaaataaatcaaaattccctaaaacactgtttggggcaacaatgtgtgggccaaagtaaatacttttgtgggctacaagtgtcccctaggctgtacatctgggacatctgatttatgtggaaatttaccgagaactagttttatttctgtggcaggtcattttcactttctaggattatgtttcttcattgataaagtgagctacttgagcaagaccagtggattgaatgccacgtcccaaggaggctggggttgtttccagggatcttacagaacttaggtgtgatactgagcatgagctacttgtgttgcattttggtgttcaaaagaaaagttctttaaatagttctgctggaaagacaaaaaaaaaaaaaagaaaaaactttcacaacaaaaatctccaaaaacaaaaacccagaaaactggcatagaagtggatgatctttgcaatttttttcagtatataaataaatgattttgatcccatttaaaattttatcaaatgcaaaaagaaacaattcaaagtatagagctaccttttcttactctactgaaatctacactttatgtcagccctggagggtttagacgcactttatgtcagcccacttctttcgactgcactatgtcagccttggagggtttagatgaggcagtgagcatttgaatgcttttaatttccatttttcaaagtacattcttggtctataggagaggaacaagatatgtaactatctctgactattgctaaaaacacaaacgtctttaataaatgttgcataaactcagaaagtgatacttcaaagtcttgtgaaaaatgatgatcaccagcatttatacagcaattagtatgtgccacgcaatttgactttattatttattcatctatctttaccaccatcttaaaatatgtgagtgcaaaaccctgagaaactttctccaactcctgtgggtgtggaaatcgaggcttagagaggttaatgctttgctcagattattaatcacttaggcagtgctacctataatatcctgctctgttactggtatttccaaacgtcattaactgtagcaagaatcctaaggcaagcactatgctatcatcttaaaatatttattgcaaacatcctatgttttattgttttatctttttaactttgagaagataaaataagccacagaagtgaaattaattgggaaatcattcgctttttgcaaaatttgggagcataaacaatgggtcatgaattacaatcaaacaaaagataaaattctaagaagtcttttaaagtggaaaaaaataactgaaaaatactgaatggagggcagtttttcatgcactgtgttacgaataaaaaatttgattcaatggattacttaatcaacattttaatagttgtaaatcttataatatttaagctgttttataagtgcctctacttataatggcacatccgtttgaaactctagcagatcatttttatttatttttttgaatttttttctttatattctttaaagaaggatacaaaattatttctatgaatatttaacatatggaaggaaatagcaataataaacataaatgctaacacatataaaataggtggtatcattaggctaaattttagtcttccaggataagtagaacatctctgacttctcaaatatccaattaataaaatgcttactataccatttggtgctttaagaacattgccatggaaacctctcaggttttatgcacagtagctataataaaattttccttcatctttcatggagctacttgagattttttttctccctttaaacatgagaaatcaaaaagaaagagaaaagaaggattaaatattcatttatccttttgcttctgacttgttatgtgggcaagtgccacatgagggagtgctgggacctcatatcaagaaaaattaaaacctacctaatgcgttccaggaatgttcagcatattagcaaattcttattaaactgtcaaaaaaaaaaaaagttttaaaagaaattccagcccctggatgcaattagaggctaccacactggatttgatgggccataaaaccattaaatctaaacactttctttttgagcctaaaaggccagaacattccaaagtgaagttttgggactcagctatgacttgaccacctattaagatgcaggtggaacagattgcagagtaacacaaagagccacacagaccccagatgactgcattagggtgtaggtgagagttttagctgttgaattttctggattttccaagattaagtgatcaaccttaacaatgagtgaaagaccattcaacaggaagaattgtcatttcctttgctctaaacccaaacgatgtattttttgaaagctttattgatttatatatttatgtgttgtgctaggcgaccgactagatatatgtttcagcatacctactaggaaaatatccccattattctcaattttacctaatccaggcaaagcactggacttgctttaaggaacatttttactctttctgaagtggagtgcctgtcatgtatcaggtgcaatgcttggactttacgttcttgtgattaatccttacaataggcctgtgaagtaattctcattctgtttgacagtagagaagaaggaagcccttgaccaaggtctagtgccagtaatggtggtgatggggtttgaacctaagtctgtttcactctaaagtgtaaccaaattttatgttttagacttgcttttctaacaataaaaagtcagtgacatgctctttctgtgtgtaagcactcacacacacacacacaaacacacatccgtattacatatgcttatatatgtattaaaagattatggacatttgatatatatacatatactaaaatgtataattcattgctaaagtattttcatataaatagtggcttcagtgttaaaatcactttgcaatgaaacaagattgttgattaaaacacctattaaaaaattagaatctagccatattaaagacagtcatcgaatggagtgatttctacgattttgcaccaaaatttaagctattgggtggctttcttgagagcatgagattgcttcttctcagaattattaatgtgcctgatgacattaaaatgtgacagtgaaaaaagtcagaggctcacatgtgtatcccaacactgaagttgttaaacactgggaggttggttgaagttgttgtgtgcaaactcaatactccttaaaaccattatttaaaggcctatcactgtgttatggtctccatatgatctgccatttatgccaggacttgacaattcagtaaaatgacagaataataacacaggaatcactgcagtagagctaatgttttagtctgttgcagagttctgccctagaaatacagtgaaaacaaggaagggagagctaagatgtccctgagactaattgttccttgaaaatattttcataagtaaaaaagaggtctagaggtgtagtggcagtgtgatcactcaagattatatagctccggattcgttcaatgggccatgatgaaagcacggcaacgattaaatctggtttcttggtctttcttggcagtgtttaaattggttcagttccataaattgtaaattaagatctgtttgacaacttttaagtatttcaagcataattgtagttgaaggtttgttcttttagatcactgacttcagaactttatttttctggttaatctcaattgtaattttagacattcataaaacaatgttgactgcgtctatgtgatggtagatcctctgtgaagacctttatgatggtagttcccctgtgaagataggatgacacactcaatggacattatggtgcacagttatacaaacacttcactatgacaggccctgagtttagaaccacacaactgcttggtacttggtcatcgcatattttccccattacgtaatgacttcctgtgcagatgacaaaatgcgttttctcaacaaaattattttcagtgcagctgttttgatgactaagttttgtaggagctttttaatcaaatgcacctaagaaaaccccaacactttaggccctttgaacatattacacttttttgcttcctctttcctctttttccttaaaaccataatttggaaatttgattctgccttcccataaaagagaattattttcaaagaaattatttgggtctaaattaacatgttacttaattgttctgcttgaatctaggtatatgattagtcccatatgaattgatgttccaaataatttactctcattgataactaatattttctatttccctctattgttttgtggtggtggtggtggctgtggatgaacatcattctcaaatatattataattcccttcctcatcaagcccagcatgataaacttcagttttgcctgatggttcatcatcttatttctgtgtgtaagattgttggatttgacattaaacatttggaaactattttataattgataacttgtgctttctcagctttgagtaagcgctctcttcttcatcttataccattttatttttatttattattcacttctgcttctgatctgagatctaggaagctggacaaatcccagataagcaagctaaacaaacaaacaacaacaacaacaacaacaacaacaacaacacaacccaaactaaaccaaaccaaaatcatgggataatggttaagtgtactgaggggccattatgcgaacacagtttaattccttggctttaaaactaataagagaagaatacataaacaaatgtggcaaatgtacctgtgaccctctccagagggtgccaggctagaagaaaggcagatttatcagcaaggcatggcgggccattggcaaaccatgggacaaccactaccaacttcactgccattgctccatatttccctcccgttttcaatgagccccaactttgctcaggacatcacacatattctactaatttggatgagtccttttgaaagaaaatatctacctcatggttctcaaagtatggtccttggaacatcagcgtcagcaggactctggagcttgttagaaatgcagatcttaggtcgcactacagacctactgagtcagaatctgaattttgttaacatacccatgtgattcctcaaagattgagaagccctgatcagagcctgggatgaaagttcctgttggttccaagccaaaggcatagttcaggtcttcacacatgacactattagatgtagatggatattgttcccttctgaagaccctcaaggtcttctgagagcctattaagttcagaatgactgcctgaaatgagtgagaagtcacaaggagactctagataattaagagatgtgttcacagtagtctttgataaaaacctgggacaggcaggcttagtatgcaggcccctaaaatttatgtacacaatggatttcctatttttgcttcttcacatccagattacctggatcagaaataaatgttttcattaagacttgatgtgacaaacaaacaaaacaaaactctgccaagctctagaagaacaattgcatttcccagccagagggagaacactgccagtttttgctgttttccaaagctgtttacctgtcctagctcatttaaatcactgtactttggagttccggattagcgtccccagaggtagctgcattcatacttgatgagttcttttaaatctcagccattgattgtaggttccatagtataggaaatttagccaaccctctattgaatggcagtttagaaaggtcgagctacacttaccttatgtcaggttattgcagacccttgtggcatttttccaccctaggacatgtgatttaactctaatagaaatctttattatgggtgggtctgagattaacttttattctataaaacagaaatcatgccactggccgtagcccattttttgagatggagtggggggaatggatgatagtaaacaaggatattaatctcatttatttttatatcattatatttatagttacattgcaaatggaagagtagagaaaccaaaaacttacactgggaactttacaatttttcttccaagtattactgattgatgtttggactatgcaagtgctgccagccccttagactcactctgcagctccccccatggaaatttgtgaacaggttagggtggggatagggaaaagcatgttcttgtttcacttcttggattatttgttccaggctctccaaagtaatgtgtaccttgggaatgcagaaattatctccttagatattctctccctatatatgtcctcacagggaattcttggaattggagaagattccactctcctttaggagctttctccataaaggtattgagcattggacactatatttgcaagggaaaagaggaatgggtctcttgagcatcaaaatcattgtagaagaatctccaaactgtttttcaaaatgtctgtactaacttacattcctgacatcaatgggttcccttttctccacaagggttcccttttctttgcatcttcaccaacacttgttatcattggtgtttttgataataaccattctaacagttggaggtgatacttcattatgattttaatttaaatttccctgataattagtgatactgagcttctttcatatatctattggccatttatatctcttcttttgagaaatgtctgttcagatcctttgccaattttttttcttttttcaacttttattttagaatcagggagccatgtgcaggtttgttacaaaggtatattgcatgatgctgaggtttggagtgcaaatgaatccatcacctaggtagtgaccacaattccaaacaggtagtttttttcagcccttttccccctcccaaccccactgttgtattccccagcatctattgttaccatttttttgaccatgtgtatccaatattcagcttccatttataagtgacaacatgtggtatttggtttttggttactacattaattcacttaggttattgatttccagctgcatccatgttggtgcaaaggacattattttgttattttttatggctacatagtattccatggtggatatgtaccacattttaaaaattcaatccaccattggtgggcacctggattgattccatgtctttgctattgtgaatagtgctgtgatgaacatgcaggtgcacgtgtctctttggtagaatgacttattgtcctttgggaatatacccagttagtgggattgctggatcgaatggtagaaaaactctcaggtctttgagaaatctccaaactgctctctatagtggcttatttaatttacattccctacagcagtgtatcagccttctcttttctccacagactcaccaacatagtattttttgactttttaacaaaagtaattctgactggtatgagatggtatatcattgtggttttgatttgcatttctttgatgattagagatgatgagcatcattttcatatatttatcagcctcttttatgcctttgtttgagaagtatctgcaaatgtcctttgcccactttttaatggggttatctgttttgtcatgttgatttgtttaagtttcttaaagattctggatattagacctttgttggatgcatagtatgcaaatattttctccaattttgtaggttgcctgtttactcctttgattgtttctcttgctgtcctttgcctattttttaattgggttatttgttttctggctattgagttgtttgagttccttatttttttttttggatattagcactcattagatatacactttacaaatattttctcccaatacctgtgttgtctcttgattctgttaattgttttctttgctgtgcagaaacattttagtttcacacaattccttaaaaaactaaaaatagaattgccatatgatccagaaattctacttctggatatttattgagaggaattgaaatcagcatgttgaagagatatctgcacttctatgttcgttatagcattattcataatagtcatgatatgccatcaacctaagtatccattgacagatgaatggataaagaatgaggtgtatgtacacaaaggaatactattcagcctttaaaaagtgggaaattctgtaacaacatggatagacagatactatatgatcttacttatatgtggaatctaaaaaggtaggtctcacagaaacagatcataaaaaggtggctaccagaggctgggagaggaaggaaaagaatgaggaaagtgacatattgatcaaagttgtacaaagtttcagtgcgactggagtaataggttttagtgatctattgtactgcatggtgtccacagttaatagtaatgtattgtatatcttaaaattactaaacgattaggtatttaatgttctccctacaaaaaaatggtaagttggtgtattagtccactttcacactgctataaggaactgcccgagactaagtaatttataaagaaaagaggtttactggctcacagttctgtatggctggggaggcctcaggaagcttacaatcatggtgaaagggaaagcaggtatgtcttacatggtggcaggtaagagatcctgtgtgtgaagtgaagggggaagagtcccttataaaaccatcagatctcgtgtgagctcactcactagcatgaaaacagcatggaggaaatcacccctatgatccaatcacctctttccctcaacacatggggattacagttccctgccttgatgcgtgggattaaaatttgagataagatttgggtggggacacaaagccaaaccttatcagttggtgaggtgatgaatatggtcattagcttgtctaaatttgtctgcaatgtatacatagatcaaaacatcactttgtaccccataaacatgtgcaattactattttccaattaaaaataaatataaataaattaaaaataattgcaaaggaaagctggctgtggagaagattaacaaataatgacattaagaaattcaggtccttggcaaaattagaaatacatacaaagctatccagaacttatttttccaaatgcattaggcgtcctctcaccttaccctttacaattgcatggcttcagagattacacagaaaacgttcagaaacattgccccagtagatgatcttgcaatgctatgaagtaggcagaacagctgtggctatagcaattgtgcagatagaacgtacttcatggatggcaagactgggactctaggacaggctttcaatccattctaccctgttgttgttctgaaatgaaagttttatctcccagtttatataggtagccttatctttgatgcttcaatacctgagacctggccagtgtcccttttagtgattgtatgtgtgtgtgtgtgtgtcatatgcaatttccttatagcaatggcacagtgtatcactgtttaattaaagaagagaaagaaatgccaaacatacgaataaagtctgaatatatctgtaacattaaaagtgtaggtgtctatctttgaagatatgtcttaaggacaatgaaagagtcagtgagtaagagaagagagtcctgggatttcatacaagatcagtgttacttgatggtgtaggctcctaggtatttcatctttaggatataccgtctattacaaaagccaagatttttagatttggatcaacattagggaacttcattctaggcaagagccaggttttgcctttatgttaatatgacctcagctgtgagctccattttgccaggcatcttaaaactgcaacacatatcattggaatcttccgttacagtctaatacatagccacacattgggagcaagaatgaaatccaacccctgtcctttgcaaaatgcaatgagacagtgtctgctttgggagcagggagtcagaatttcattgtggacaatggataaggtgagtaaaagggcttaaaacatttgtgctttcaagccataggctaggataacgatagtcagaactttttgatgaagtctgaccatgctacgccatttataaaattttgaagcttgtaagtattaccccaaaatgagcagtgtgaactcaaagggtttatcattgtctctcaggcaaaggtaatatttgaattatttagcaaaggactttgagcaattggaagagatactcagctgctggtctctagcgctctaacagggtggatgccccccgctctgccggcactgatgtttaagttgctggattatgaggaagtctggggattccttggggagaaaaggaagtgatgacatattgaagcacaacgacatattgaagagactcgggggctggggtgataaacttcagagccgtggctatttaccaattggagtgtaagtattttaatattttaacaaacataattgccattctggtatgtaccaacttcatctcagatctgtccttaagaaataggcaaattctttattgcctctctgaatggttcatataaattcccaggctcccttagctcattctaacataaaactgtattaaaaataatgaatgtaattcatcaataattttcctttgtcatagcaaatagtcacaagtggattgagatcagagtgatcactcatatttgttctggggagaagggagcctgctgttttgctcctgttttctcctaggactagtattttagcttcaaatgataataccttagcacagactctgatattcctcctacatgcaggagcattctcttggaataattttggggatgccaattcaaaatttcagccatgtatgatttacttattggaaaataatcactgagcagcaataactccagcagttacttgtatcaaggtagaatcaagaaatagatggtatggaccaaacttgcttctctctaaatatgcatacccaagtgatttgggtaaaatgtttgtgaagggcttacatttcctgcaagtcagatggtttaagagaagtagaaattatgtgtgttttgcagcattttggtaatctgtgtggagtgtctgtagatatttctcatgagttcaagggaatccttttgtggattttgatgttcctattggcagagctgctgcttgactacatgatgtctttgtattaactacaaaaacatgccctatcatctgagtgattttctctgccagacccctttgtgcatccacactctgcacctccagtgtacggaggaccttcccactggattctaagattccatgccttcccaatgcatggcagtgtctctcatgcacatggcaaacctactctcttggatgtcactgccctgaaatattgagggagtacatttatctaggcatggtaccagggagtcatttagacatgtagggagtctagaaagatcattgccctgggagagtgctcagccatgctgagttctcctactttgttgctcatttctgtgtgaccttaggtaacatcctcttcaggactttttttttttttttttttttgacagggagtctcattctgtcatccaggctggagtacagtggtgtgatctcagctcactgcaatctccgcctcctgggttcaagcaatcctagtgcttcagtcgcctgagtagctgggattacaggcatgcgccactacgcccagctaatatttgtattttcagtagagatagggttttatcatgttgatcaggctggtcttgaactcctgacctcaagggatctgcctaccttggcctcccaaagtgctgggattacagatgagagccaccaaccctggccaggacataatttatttcaggtgaattgattgttggaggattttgatccaagcaatcaatgtcccttggtgttcctttcaaacagcagtaagtgacctgaatttattttccacatttccaaatcttaatgaaaatcagacaatggtctatatgttcatttgtgttcttacttaataaaatgtgggttttagacaatattttgccagtcatgaattcctatagaaggaactctttgggagaacagactagtgatctatagacatgatgacctccaactcagatcttctgtagctaaccactgaccgggagaacatgtatgaaaaacatcttcaaaggcattgaaaaattaacatttatcaaaaacaaaatacattttatttcatttgaacttagacctttactatctaatggctatggtactatttaaatgtcaaagtgtgatctagcatcagcctaatctggttagaaatgcaaactcttgggccacatctcagacttactggaccagaagctctgtgggtgggacccagaaatctgtgtttcattcacatgccctccaggggattgtcctgctaaagtttgagaatcatggaagctttttaacctctcattatagctttataagcagcaactcactggattcctatcaacatcctgtgagtgtcatttggacaagtatatttatacccatttgatgcatggtaggcacacagatgagtcaaatgacttgaaggaatagagttttacataatatacttttatatatttatacttctaatatatttatactttataacagatttgactgttttatatattgcatataaacattatatcagtttctcctccactaaggctgactccaattttactccaattttactaccaatttttggaagaaagcctacctatcactcatgttctctcaagtaccctctaaaactattagttagatgactctatttaattttccatttatttgcccgtttcttgctacctttccccccaaaatgtaactgctaccttgctcaaaaggatgtgtctacttgggatatctagcacacacattttatgagattttaaaagacaacataaatggtaaactatatatttaatacaattttgaaagacaaaattttaaaattaaaaaggaagaaaaaaattaaactaaccccataattctcccacccatcattagcatgtagtttgtttagattcatctaccaataagtagaattgtacaaatttgatatcatgtaatacatgtcattttgtaaacttttttctttccttaatatatctatatatcataaacatttttctatgtctatattattttaaaattgtaatacccagagttctccagagaaacagaattaataggatctcccccttggagatttctcatctttttctctctcgatagatacagatagatacatacataagtctatctctctatctctatctttatctctaaaacacctatccatagatagacatttttaggaattggctcatgtgtttgtggaagcttgcaagttcaaatgtgcagagtaggtgggcaagctaccagggaaatgttgatgttgcagttccagtctgaaggcaggctccttgcagaattcttctttttcttagcagtcttacttccccttcctcttccccttctccttcccctttcccttcttcttctccttccccttcttcttattctccttcacagacttatttttaaggccttgagctgattagataagacccactcacattatgagggataatctgctttacctgtagtctactaattaaaatgttaatctcatctaaaaaacaccttcatagcagcattcagacatgtttcaccaaatatctgggcaccatggtttagcatattgatgcagaaaattgattatcataataatattatttttttttgagatgtagtttcactcttgtcacccaggctagagcgcaatgctgcaatctcagctcacttcaacctcttcctcctaggttcaagcgattctcctgcctcagcctcctgagtagctgggactacaggcgcccatgaccacgcccggttaattttgtgtttttttagtagagatggggtttcaccacgttggtcaggctggtctcgaactcctgacctcaggtgatccacccgcctcagcctcccaaagtgctgggattacaggcgtgagccactgtgcccagcaataatattaattttaatgggtgtgttcatttcattttatatatgacctacaatttaaccaatcccctaaggctggatgttcaggttcttaatattttttgcccgtatttacagacacctttgactattggatttattttgttcttcaggaacaatatacaaagtgtggaaagaaatgtatatttctaatcattggaaaataaacactgagcagaaataactccagtagccatttgtatcagaggaggtagaatcaggaaatagatggtatgggccagactttcttctctctttaagagatttgacttcatattgccaaattgcccttctagatgtctttactcatccaactacaattcaaaggtttgggagggtaagcaatgccaggcccatcttgatcatcccctttctttctcagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggtaaccatgatcatgtgggccccgagctgaggcgaaagggatcttgactgggaatgttagggtctgggttctactgatagcaacgttgctaaacatctagttaatcttcagctaatcacatcccttttgtagacatcactttttttgagatacacaatagaaacagaaatggcctctataaaagtccaataaattttcagaccagagtgcattaagggctttggctttgggaagtatgaattgctatacagatggaagatactgaattttgcccaagcagcagtttattattatcatcctggtgccctatttctttgttaaagtcaaagagccacctttaccttttatttttaatggtacatgggacagctaaggctaagaagattgaagaaagaaaataatgaaggtttaaaaaagccacatctttgatccctcactgtctacttcttctttcagcaatattcctttcactgtggttcatccatgggtcaagattcattgattcattcactcaaatcattcatcttagcaaaaacaatatatcacataatctgatgttgaactataaaggtttcatcaggtcattcattcaccctgtccacaagctgtgaattattatctctttcctggttgtattttgggattacaatcatcttgagtcaaagctggaaactgagtggaagtctctgggaaagactcaaacctccttaagctatacacctcttttccccatcagattttccttccttcagtttccaccaaaatgtgctcttggatttttcatatgaatgtataatgtacctcaggcctataagtattttaaaagggatcaaaatcttagttttaatggaggacatttttatgatggactcctacagcatccatcagaatatgtaagatgatgaggaatgtcttcctgtgttcccagatctcatgccacagaggcccttgcttactctatgtttgaattgtatttggaaaaaaaaaaaaaaacaaaaaactagggctagcaaaattgaaaaaagataaaagacgaaagaagccacatgtaaacatactgtgtttactcttctaaaatattaaaaaatgaaaagatccaaaatcaaattaatattcccctggaatttcatatctatttcagtgactgtggagtgaatctcaccacgaaagttgctgcagtcttgtataagtttcacatagttttactgtgtttgtgcctatgtgagaataaactactgtgcataaaatcttgctgttgagccatgtgtgaattagctgtgtgatgttacctccctgttactaccaggctggtttaggatatcatttctgtatgtggcaccaggattagaccaatgacagaaaaagaaagtgctctccctgccaaactggccaataaaactgttccacatatcccagactcagggttacctaaacaacctgtgtttaaagagaacaaaaacaaaagcctctgacatagtcttactccttgccaaattcgtcagaaagctgatggattcaaattcccccaatatgaatcccgtatttacattatttctctattttgactacttttttttttttttaaagactttctaaatagtttcccactatcgaggcttcttagaggaaacatttctcattatttccccttggctatttgaaaaggaatttgttcttccttttcctccatctcttaacactactactactaacaatagtaacaacaatagtaagtacagtagggttttttgttttgtttttaacttaagacatactttcttgttctggataccaaaatatgtttcacagaggcatctacttagatggggtgcagatgacacagttgttaattctggcaggtacctcttgcttcttcactgctggggctactcagtgagtggcaggaaggttgatttgctttccccccttttcttttgctcctgggctccttcccagatgatgtgacgggccatgaaacaaagactcttttcagctgtcggtgtgcatagaactggctgcggcttcctagcttgtcacatctccggtctgaagatgatcaaataatgagcaacacatccaggttatagggaacacgggaaacaccccgcagctgggtgtaccccagcccctcagagtgcacattggtgttgtttgtcctagtggacttcggagtaggccagtgccttctggtcagttcctcagtggcccacattcagctcttaaaggcagagcatgctaacgggaggtccaggcttccgcctgaggccaaatacaccccaaaagctcatctgttatagcctgatatgaaatcggtttctttctgcaactgacctgactcatagaaagtgaagcctggcttttcataagtgaagtttggcaggcaagggaggcaggaaatccagaggagaatgagcctgtaaagcatggctccttccagcccttgttacttcctctgcccaagtgtgggggagggtcctgtctcttggcatctgggcccagcaagagttcagaggtttggtagtctctgcttggtccatatgcaaaacacatgtatgtgtatacattattaatggcaagggggttcctgaaactgagagggagtaaggagacttctcatctgctcttggaagaagcaaggaatgaagccagttcagtagactgattcctgaggctttggggcaggaactttttctttctccatatccccatggagatggttcatttaccctgaattaagatttggcccttcggtgcagtgccaaggcagtttaaagagaagaaaagtaatttctgatcattgactaagatcaaggtaaatcatgacacttatcctttctatgatttgccagtgacatgttttcttaagcccagaaatgatttattgatcgcagcagccagaatatatcacactaaaacagatcagcctgccactgtcttctcaggtctctcatgattaaagtggcctgctttaaagtagactcaatgtgaataggtctccatgacctctgcctcactgcgtagcactcacatcctcacccactcttgcactctggcttccctgcggttctttcaatatgccaggcatgctggaaccccggagcctttgcactggctgttccctctgtctgtaacagtcattcgcagaatcaacgcatgactaatagcctcacttccattgagtcttgacattaggaatggatatacatgtctatattgggaaaccacaataaaaattgatggtagagatgcaaatatgagcaagataacagggtgggggcaggggagagaggtcagtggaggacttggcacagtagcctcttaaatggcacaatagcctcttaaatttttggttaagaaatcattcacattgataagtatggcaggataaaggtgtccatgagtgaatcccgggaacctgttactttatgtggcaaaagggactttgcagatgtgattaacttaaggggcttgagatgggaagatttttcctgtttttatcagtaggcttgatataattaaaagggtccttataagagggaagcaagagtgtcagagtcagagaaagagatgaaatgacagatgtagaggttggaatgatgtggtcaggaaccagggaaagcagggggtatctagaagctggaaaagacaagggaatagggcttcccctagattctccagaaatacagccctattgatatcttgagtttagtccagtgagacttattttagacttctgacatttggaactgtaatataataccttcatgttatttttattgctgttgtaacaaataaccacaaacacagttctactaatttctttctcaaggtagcttctcaattttgccaacgctggttaccatacatacttaaagtttcattttgagtctctgaaaactcacatctctcttaatctgctctacttttttcttggctttgtatagtgcttatgttctgctacactttgtaatttattgattatgcttaccatgggcagggattcttaactgttttatttatttatatatcttaaacattgaaaacactggcatgtagtagatgcttaataagtaattgttgactcaatcgataaaatatactagaacatacaagattttcccaatgtaacataactagtaagaggctgaaccgggatttgaactcaaaattcattccctgaaccttcttctagcagccacattgaggaagaaattaccagggctgtgttctcaacacaagtgttttccgaaccacagaattaaaggctggtggcccatgtatcagtgtctgtatttatgagcccctctttcaatctctttcttttcatattgtgttgatgctgtagcttctactggtcatgttatttttttgtttcccaagacggaattatgtggctttatctttaatgttgcattatcaatacttataataaataatattatgtattactcaatattcatgattaatagtgttactattggttatttaataatgtttaacttacattagcagttgttactatttttatgatgctaaattactaacagctaaaacaacttctatattaaaaagtatatttgagtgccactcaagagataatgagtaccttacaaagaagaaatcttgtttctcacctttgcgtcattaaacagatcaggatttggagaattaagccctaagtaatagtgttattattttgatctcacccctttttttcttatgaaatggaatactttggttatcagaagccactttaagcatatatatatatatatatatatatacatatatatatatatatatgtcataatccgaataaaaatagcattcatggaggtttcttttggagcctttggtaaaacactccatcgtgggtctctgtcaagatatctgaaaactttttcttggcttctggctttgaacaaagtttcagagtaacaacaaggcttcattgtgcactgaaatttctgtaaggcaacattcattcaagtgttgattcgcatttcaccatccaagaataacaacagttatttatataattttatccacgtttctgttttttcctatccatttcaccctttcaccccacccctgctgaaacactggagcttgtttgggatgggggtggggtgccatgcagactacatacacatacagatgtttttctttttcttttcccggtcttgctatgggatagacagactggactttttcttattaacaatattatttaaaagcttggaatttattatcatttaatcatttgtatgtaatgaaataggtctccatggtaaagatgtgtttattgaccagcggttagctttattcaaattagggtgaccatagaagaccaaggactatgatataatgtacaatcctaagtggtttgatttaaataaaaagaaagaccaggcatttcagctaaaatccccaccaaagcccaatgactagatgggcatccatatgactcaatgaaattttctatgatcttaaatggccatctgagtccgtgaaactataggactaactattcaatccttattgagaaagccttgttaatagcttgaattgagttatatgggataggaatgttcatatctttatgacaatatatgccacctaagctacataaccagctgtgttagctaaaatactctaaagtgtaaaaaatcatagttttctattaaaggaagtcatgattgttaaaaataatttttaaatagtgtgcctagattcttctagtataatatataattttttttttttttttattttgagacagagtcttgctctgtcacccaggctggagtgcagtggctggagttgctcactgcaacctcgcctcccgggttcaagcgattctcgtgcctcagcctcccaagtagctgagattacacgtgcccaccactatgtccggctaatttttttgaatttttagtagagactgggtttcatcatgctggccagactggtcttgaactcctgacctcaggtgatctgcccacctcggcctcccaaagtgctgggattacaggcatgagccattgcgcccagccgatatataaatttttatatggctccatgatcttctctacatttaatgacagaactggtggaggggaagaaagagatgggactaagccagagatcaatatacatacaactatactttgaccaaaaaaagggagattgactggcaggggaattaatagtatgcagaagagcaaggtgagtccagtcactgtcattattcaaaaacagcctttcaggagaagtttgcaactgaatttgggactgtgggcagataagtcacaggaatgattctattgtgtatcctgaagtcatccatccagctaggagtcagaggtgcaggctgaaaagacattgcccctagagtggggaactgccaaaatctagccaggatattaggccaagagaaaagacctcaggcacaggggaagccagcttcagaatcttagaggtgagttagagaaatactgggctaagctgggtcaacaaaaatagtcatacgggaaggaagcaactcagtggtactttgtctcaggttgggttctcctagaagcaggctgtgagcctatgatttcagtgcaagtagttaatagggaaggtgaagggaatactgctagaggagtaggaaagtgaggcaaaggagggaaggcattgaacaaagggcataataccaagacagctacagtcggggcaactgaagcttaaccctgctggaaaattctgggaacttaggcagaatttatctcagagttagtctacctagcggtgagggagctggggtatttacacttcaacatccctaatgcctccagtctgctgtgtggattgtgactcaggcccaggagcaagaaaaaaccactggcagagaaatgaaggtattgataattgaaaagctcttgtcgttgtcaaacttgtctggaagattagagaagtttgagtcagcagaccgagaagttcagagagaacaactaaaagggggagggaaaggcaagaggtgacaactcatctgctgggaaacagttcccacttctcccatgcccaccaccaccatcactactgcaatagcaggcaagaaggagtctacaggggcaacagacagtcatgttaacacctgttactgcggttccaacctcttctagaattttctacaagggcattggaaggtttcctgaggtcaggtgcaataaccaggaggaaactgagatctgagcaaagagaatagtgttaggaatgaagtcaagggacccagtttggagctcaaatactgctattagctagttctatatttgaggcaattcattttgtacctctgtttttattttctatgggaatcatagagtatcaatgttactttctaagagtcttttaaagaaaaattgtgataattttggcatacaaaaccctttgtaaattataatgcaccataaaaatgctttttaaattgtgattaatttgcattgctggctgaaaatcgtgtcaactctattgtctatatccataaactggcttaaaaatgagatgtttgaatatgaacacatttttttttcttgaaacagatttgtttgtgtaaacacagcctaagatgtaaaataaaatttaggtcactgattaagaccactagagtatcacatttagtctagagtcctacatcaaaatatggaaaatatgtgtgcaattgacttatagataaatgagcagtgaacagccaattgatttgaaaagggtaacatttttcattgaatgaatcattggaaacgtatttctaatttggcaaatttctcattttatgcatttcttattttaggatgaaaaattttgaaaccagtactttatcattttctcctgaggaaaattttggagacgacagtggacttgctgcatatgtggctaaagcaatagacccatctatcagctgggaagatatcaaatggctgagaagactgacatcattgccaattgttgcaaagggcattttgagaggttcgtttatttctctacttgaattcatactgactttgtgatcctttgtggatacgttcataatattcttaaaggaaaataacaaggaaaaattaacatggaaattgagagagacattccaactctcaattctctgttttcatgttagtgagtaaatatttcttcattcttaggtaatattctgaagcagagctaaactctcatgaagcacaaagtgagctttttcaaagaattgggatgctattgccttatttcagagctgttactgaatcatgaagcctggcaagatcttggtagaacgtcatgagttcattgctttacctgaaagctgcaacagttgctctcagaccaaagagaagccccaaggagatattatgacagacagacttgttttaagagcttttactctttttcttgtgctccctccagtacaaatggctaggactagtttttgagtgtggtcaaacccacatcacctctctgaagagaatggctgactagtatggtcaaatgctcaattatcaagccatgggtaataagaaacttaactatttcctccagcatccccatgtattcagatcccacagggagataccaatgactataataattttggagtgtaaatgtgtttacccacaaaggcaagaaaacctttgtggtatataacctttggttatatagataactgccaaaaacttttttttaactgaaatgtttgagtaagtgactttaagatgagagtctgtccattaatgtatgtctattaaatgactttgaatcaaaactttttcatagattttacttgaacatgtctgagtcatgtggtccctatttataaaggtctacctactttgactatttaattttatgtgtgaagaaatgagcttataaaagtaaaagaaattaagcccagtgcagcatttaagtagtggagatatgtttaaatccagatgttcttattctgtgcttaaacaaattaaattagttttttaaatcgatatataatagttgtacatatgcatagagcatatagtgatgtttcaatacctacaatgtgtagtaatcatatgagggtaattagcatatccgtcatctcaaacattattctgtacttctacaaaaattgttcactgtttcctactaatcattgtcctagttcaaaatttttgtggattatacttcaatgaatatgtattggtttggtttacagacacctttaaataatggtttcacttcctttacttgcatccatgttgttatccatgattactagtgctacttactgtggctgcattcaactgtggggactaaataatattaacctgaaggttctgtgccattcatttatttattcattcatccattctcttattcactcactcattcattccataaatatcataactactgtgtgctaaaatatttttatgaaataaaataattttagatccttggataaaatgacagtctattcttgtaggagacagttatataaacaagcattttgggtgcaataaaatgttgcatagaaatctatgggcatggcttaaaaaagaatatgggcaataatgtaagagtgggttagaaaacacttttcagaatgaaagatgatgcttgagttttgccatgatatcaagccaatagactctacttcagactgaagtactggcagaataaagcaattatgcataacaatttataaaacacataagccatttagagaatttagactttaaagtggctgagaaccactgaagtgtcttgatcagagaagcaacctcatcatataagcattttacagaaatcatgctttgcacaacggacctaatggatggaggtggcaaaattgtggtcagggaagacatttaggaaatgttgttgcccagaaaagagataattgtagagatgacctagagtaatggcagggaggggactgagaaggtataattaaattagaaaattaagacagttaatcacagagacggcccaggggatctggctttaaaacactaaggaagaatagaattttagtcttgttccataattgtttcaaataaggaacaaagaaggaggtcattttattttttaaccaccttttcaatctaggatttccagtgtatatcattttctacaactttattccttaagatatgtgcaaatcaattgaattagatttactatcacatgttatgttgaaaaatattttggtttctagatcttcattctgttatcactttagctaggaccactgccaccaccaccaccaccaccaccaccaccaccaccaccaccaccgctaccacaatctccattaccaggacaaccaccagcactgctgccacccaccaccactgctatcaccatcaccactcccatcaccattatcaccactcccattaccaccatgaccaccaccaccaccacaactaccaccatagccacaacctccatcaacaccaccattaccaggaacaaccaccaccactgccaccaccaccactgctatcatcaccaccaccatcaccactcccatcaccactatgaccaccaccaccaccagcgccataaccacaaccaccatcaacaccaccattaccaagacaaccaccaccacctccaccaccaccaccagtatcaccactaccacctccaccactcctatcaccaccatgaccaccatgaccaccactaccaccatcatcaccacctctcttttcttctcatccatatggatttctggtttgtttctaaatatttttgtgacattttacacatagtagatagcccaaatatttatttactgaacttcttgatgaaaactataagtctatctaattagagaaagctcaaaaaaaaaaaaaaagataaggagagatgggacatttgacatttaaagggaaataacttatttatacatgtacagtttacatcgtctacttttctgtaactatttggttcttttatttaacaaacaagaatcatctctatgacaacaattgcaccaagtaacacatatgctcaatctggatcggaagggagcagtggcagggccagttctgggaaatccaattttctccatttcacaggtcctcatgcagaaatcacatggattgactgagagtcacccatcctctttcattttcctttttcagaaaagtgcagattatattttcaggaacatgaaatcatgagcaactgtgaagatctagaccaaacatctgactgtctcaccattttaataattgtattttaatgaatgcaaattagaatggaagatgtaatctcttactgtgtgtggttaaactgcccttagagaacttcaacgcaagctcctaacatgtacttaagacagaatttggtctttgtcagaactcacactagcaaacaaatggcttttttcagcaaaggcatggtgccttttagagttcttttctttttcttttaatttatttaaattaggctgtctcacttctactgtggctttgtgtttctgagaaacttttctctagtcagctcttcatatacaggcatacctaagagacattgtggattcagttctggaccaccacaataaagcaagtcacataatttttttgtcttcccagtgcatatgaaagttatgtttttgctatactgtagtctattttgtgcaacagcattatgcctaaaaaacaatgtatataccttaattaaaaaatacgttactcctaaaaaatgctaatgatcacctgagtcttcagcgagttgtagtctttttgctggtggagggtctttcctaggtgttgatggttgctgactgatcagggtagtggttgctgaaggtttaaggtggctgtggtaacttcttaaaataagacaatgaagtttgatgcatcgattaacttttcctttcaggaaagatttctctgtaccatgagaagctatttgatagcattttacccacagtacaactttttcaacattggagtcagtcctctcaaactgccactgctttatctgtgaagtttgtgtaatattctaaatcctttgttgttatttcagcaatgttcacagcatcttcaccaggagttgattccatcccaagaaaccactttctttgctcatcataagaagaaactcttcattcatttaagttttatctcatgattgcagcaatttagtcacagcttcaggctccacatctaattcggttatcttactgtttctgccacagcagcagtgactttctccaccatagtcttcaactcctcaaagtcatccatgagggttggaatcaacatcttccaaattcctgttaatgctgatattttgacctcctcctgtaaatcacaaatgttcttaatggcatctagatggtgaatcctttccagaaagttttcaatttattttgctcttattcatcagagaaatcattaactatagcagctataatgttattaaatatatttcttaactaataagacttgaaagtcaaaattactccttgatccatgggctatggagtggaggttgtgttaacaggcataaaaacattaatctttttgtgttcttccaccagagctcttgagtcactaggtgcactgtcaatgagcagtgatattttgaaagaaatctttttttttctgagcagcaggtctcgacagtgggcttaacatatttagtaaaccgtgttgtaaacagatgtgctgccattcggactgttgttttatttatagagcacaggcagagtagatttggtataattcctagggttatatttgcagccatgaatagacttctcctctctagctatgaaagtcctagatggcatcttcttccagtatgaggaaggcttgtttggaagaagatgccatacagcctgtttaatctacattgaaaatatgttgttcagtgtagccacctccatcaattttcttagctagatctcctggataacttgctgcagcttttccatcagccctcagctgcttcatcttgcacttttatgttatagagatggctccttcctttaaacctcaggaatcaacctctgctaacttcaaactttttttctgcagcttcctcacctctctcagcctttatagaattgaagagagttagggtgttgctctagattaggctttggtttaagggagaatggtggctggtttgatcatctatccagaccactcaaactttctccctatcagcaataaggctgctttgctctcttatcatttctgtgttcactagagtagtagctttaatttcctttaagaactttttgtttgcattcacaacttagctgtttggcacaaggagcctagcttttgtcctgtcttggcctttcacatgccttcctcactaagcttaataatttctggtttttaatttaaagtgagaggtatgtgactcttccttgagcgcttagaagccattgtagagttattagttggtctgatttcaatattgttgcttctcagggaatagggaggcctgaggagagggagagagatgggcgaatggccagtgagtggagcagtcagaatacacacacttattaagttcctcattttaaaggcgatggttcatggcaccccaaaacaattacaatagtaacatcaaagatcactaatcacagatcaccgtaacagatgtaacaatgaaaaagtttgaaatagagtgagaatgaccaaactgtgacacagggataccaattgagcacgtgctgttagaaaaatagtgctgatacacttgcttgatgcggggttgccacaatgccacttgagcattgtacataccttaattaaaaataccttgctgctaaaaaatgctaatgattatctgagtcttcagcgagttgtagtcttcttgctggtggagggtctttcctagatgttgatggctgctaacagatcaaggtggtgatttccaatttctaaaaaactcagtagctgcaaattgctataaagcaaagtgcaataaaacaaggtatgcctgtatacattgttcaagatactctaaaaaaatatgcgcttaatgttcctggattgtgttttaaaattgctgtcatgtgtccgggatttagttactactctcctctgagtcagaacagattcctaggccaaaagctccaagggcttgagctcaaatcttgagtacttacgttttgagtacttgagtacttatttttcacttttataccttgttttaaattaaatatggaaataaaatattaataagatagcaaacaatgccactgcccattgacgactgtgtctatcaatcaatgtacttaatggcaaattcacaaatatggaatgccacactcttaaactctaagaattaataaaaatatccaagattcatgaacattttgctaagtgggaatattttggccctttcattaaggcaaaattgcttttctctctgtgttaaaataaattgtaaaactgtggacgaggctgcataagcagctaataaaaggggcttatatctggttataaattatccttattgtctttgcttctctagaaattcacaatcctatcctctgagcaaatcattatgtcatttctccctatattcccaatactacctgaagaatataaaaaccacttgaaatgatggcagataacagactgttacttaagaaacatttaattcaaatgggaggcactttatcatagtaatcttgactaggataaaactgttataaacagaacccaaatagagttgccaaacgcatcctgcaaataaatctttctccacctactcatctttttagtttatattccctgcaccatcagaggaaaatggtaataatagttaatatttatctgagtgttcaacatgtatcaggcactttctaaacacacattcaactcatttaatccacaagctaatcatatgtggatctctatcctcattttatggcttaggacaatgatgcaccgagaagttaagaaacttgccacttacacagcaagcgagcagcagatgtgggatttaagtccgggtggactgatgacttaccttaagcagtgaatcacttcttcctcctgtacaggatctgtttattgataatatttaatttctcataatgcaggttgtagctacatggtggcagacacaggaatataaactggagagctatacgtatgctcaattacgaggtttgaaaaaataccattgtctatgtttaagtggttttttgtttttttttttttacatttgacttgaagttcaggatcctgtacataactagataaccaggcaaaccacaccaattcagaatattattttatactgtttaataaggttatttatgactacacctaggtttttaaaataaataccatgctatgtctattaggacaggtaagctcatggaaacattgaaggtgagctgtgcaatatgacagctatataagttaattaaaatgaaatagaggcaaaattccagtttcttagtcacactgccacatatcagttggtcagtggctacatgactagtggtactgtatgatggaagtgcacagatctagaacatttttattatcacagaaagttctatggtagtccattgaggccctatctttagagaaccatttatgaaatacgtttgagatgaaagtgtactgggcttctggtttaaaatgatggactgagtacatatgaattctcttgttaaagtcaagactccactgaaattaaagtacagaaatgcaaatatgtatacaccaatgacagcaaagcaaaggaggatagaggacagaaacagcagagaaattttaagaaacttttgaaagcctgttgacagcaggaaaacatacagaaaactgtaccttgagataggctttcaggaaatcctagaggaggtaggggtagattatgtaatagaatgcttgagaggctgtaggtatatagtctgcaaggatggcagagagggtagtgaagacgaaacgcaaggggactgactgaagatcaatataggaagctgttatgcctgccagcaatcctgctcccaccctacttcttatgcaacagtggacacttggcactgacttctcaataagaaagtagctgatcctttctaaagacattaatgaatggctgggatgaaatatgggtgggtggcatttccaatatgacatgatgctgtcctgattctgacacttgaggagctcacagtctaattgcctcttagttgacatttcctgcattttcttacatggagttccaagaaagaatttccactccagacaaggacttggcaagaatgaaactaatttacaaaacagggcagttcctcaatctttcttccttaaatataaatggaaaatttagcatcactaggcatatcagaaacaccaaaacataacaaatcaagatgaatctatagaaaagcttatcccaggaaaaagagataaaacaagaaaaagaattttaaaaaatccaaatgatactcaaaaaagttgaaaatatacatataaataggaagagtctgccatgaaaagaaaattagagcataaagaagagttcccaatgattagaaaaatgacttctgatttttttaaaaaaggggaagtcgaatataaaaaagagctctcaaagattaaaaacatgattgctgaaatgactgaatggctgagcagtaaagtcaataacatttcccaaagggtaaaacaacaatgagaacaataagataaatagaaagaaaaaattattacatgaatgtacatgaagacaactttaaaatgtccaatatccaactgataggagtttcaggtagaaaagtaaaagaaagaggcctggaggaaattattaaagaccaacaatttccgaatgctgataatgtaagtcattagagtgagaaaacaaatcacaacttatcacgttccttatgaaatttcagaaaagtaatgtgtataaagagataaaataaacataaaactgtttacttataaaataattcaaatcaaactggtgtcagtccttgcatcagcaatgggtactagaagacaatggactgatgtctttaaggttctgaggaaaatggtttttccacttagattattatacccaatccaattgtcattcacacacaagagaagctatttaggtggtctgtaagtttacctcccttggatcctttttaagaaagtaacaaaggaatctactacaaacaaatgagaatataaaccaaatgagggcatactcagactacaagatattcaactcagacagaagaagagcaaactcaaagctgaaggcagacattatgtcttgtgagtagccacaacagaatggaacaggaaggcagagggctccaggggttatcttcaggtggggaataaggtgacctaataggttagatcaaataattgaaaagtttgaagaactggataatattctgaagtttcacagtaaatggaaaaaagaaaagaaaagaaaggcagttagaaatctaatctaatcctaatcacctcccaaagaccccatctccaaatgccatcacattggggatcagagcttcaaaatacaaattttgagaggatataaatatttagtctatagcatataggtttaacagtttcctatacctataattttaggagaaaaatatcactatcatcatttgaaaaaaataagaatagttgaaaaatagccttgtataaaacaaagtttggaaaatgatagcatataatttataaaataattatcttgtcttttaaaattattcttagtcaagtctgcccattatctccattgcttaattttacagattaccaatgacctatacccatttgcttaaatttccaaggcctgatttttgctacgctcttgaaccagttggttttgcttagtattgcattttgaatgttaaacctgagaaccagtgttgttttcatgattcttttagcaaatggtttgttattaggacatgctaaaaataatagtttagaatataaaatatgtttttaaatgttgcaagtagatttaagagaacagtaatctatttttaaactgaactttcagaaattaatatattttaaaacatgattagagatgtagcatagaattttaatttttttttttttgagacggagtttcactctgtcacccaggctggagtgcagtggtgcaatcttggctcactgcaacttctgcctcccaggttcaaacgattctcctgcctcagcctcctgagtagctgggactacaggcacctgccaataggcctgaggcctggttaattttgtttttgtttttgtttttttgtatttttagtagagatggggtttcaccatgttggtcaggctagtcttgaactcctgacctcgtgatctgcccgccttggcctcccaaagtgctgtgattacaggcatgagccactgcacctggccaattttttatacatttcaatgagcaaacatatatttaattatagaacaatattacttaaaatagttactaaagctattacaacttgttattaaagattgttttttgaaataggtttgtccactgcctgttacatttacagtgcttgacatttttatgttttttttttttttggctatccaaattgttgaggaataattttgctaaatgataaaatggtataatcttattaattgaaaatatttagcaagagtatacccttataaagataaacacgctttatgtcaatgtattatctattgatacttttgtatattttacaattaaattgccgtaagtagtaacatttttttgttgtttttggccaagaaaactaccactaatattctagcgctaaccctagggtatacttttatgctaagattattgacccaggtatgcagatgggagtcagcctccatttgactggaatgtggaggcctctaaacccaagctgcctgttaagttacagtttccctaaggtgcttgttttactctctccaggtgatgatgccagggaggctgttaaacatggcttgaatgggatcttggtgtcgaatcatggggctcgacaactcgatggggtgccagccactgtgagttttggcagacgctaagatttccttttggagttcccatttccatcactgtgttactatctctatgtcttcctcctctctgtgtgtactttgtgtaatcactcaaggtgaaatacgtgcaaatatgtgagatcttggattttaagtttagtggcacataactacccaagttaaaaatttatttatgtttaattagcccagtgcatttatttgtaacctatgaaagtctctgttaaataataagtcatttgtgggcaaggattattctggcattcatagatacaccaataggtatgtaaactggagtatgaaagaaaggattatgccatgaatgtccaaggacatgagctaacagagaattgtgggatttcaaaggctgtggtagaatctgagatcatcagtacaattgagacaagaaatcaggaggagggacataatcagaatttcctgtggggtggcggttgaaaagctgcagagaaatggtggagagttgggaaaaggagaaaaaaggcttaggtccgggcaacgggaagtgagctgcaaataggctactctggctgagttgtgaaccaccttggctgcacattggaatcatctggggaactttaaagaatcctgatctgctccccaggtctatttatcagaatctctggttggggcttaggggtgtataggcactagtatttttttaaagctatcaggagattccaatgtgcatctttggtttttatgcactggagtaggcaaatcagagtccaaaatctagatgtaaaagtgaactaaactgtcctgaaaggtgtgggactgaatacaactgtatcgaagagcaattagaccctgaagatcctaaagccaagaaaaccgacagaggcacttagggcagattttaagatcagagactgggggtccagaaatggatccgaagccatggagagggacacatttcctggggtggattttatggatccatgatccagaatgggggccgggggccgttggtggggtgctatcaagtggttcccttatgtgctgtccacttgtggggatttggccaagcctcttgtagatggagcaatgatgttgcagatattttttgcttctgctctggccccatgtggaccctctttgcagtgtgataagcctaagagtaaaatgggaaagagtgagatgtgctcagggaatactaaataatccatcttgtttgcagtgtctgatgctgtaactgcccaaccgtttctccttgtccactgtatagacagagccaatttatcaagacggggaattgcaatagaggaagagtttaattcacgcagagccagctgtactgggagattggagttttattattactaaaatcagtctctctgaaaaactgggttttagggtttttaaggataatttggtggggaaggggttggaaagtggcgagtgctgattggtcaggtgggagataaaaatcatagagagtcgaattgtcctcttgctctgagtcagttcctggttgaaggccacaataccagatgaaccagtttatcagacagtggtgccaactgatccaggtacagggtctgcaaaacatctcaagcattgcttttcggttttacaatggtgacgttatccccaggagcaatttggggaggttcagaatcttgtagcctacagctgcatgactcctaaaccataatttctaatcttgtgactaatttgttagtcctgcaaaggcagcctagtccccaggcaggaatggggttttgctttggtaaagagctgttatcatctttgtttcaaaattgaagtatgaactaagtttctcccaaagttagtctggcctacactcagcaatgaacaaggaaagcttggccgttagaatcaagatggagtcagttaggcgagatctctttcactgacatatttttctcagttataatttttgcaaaggcggtttcagtgggagtgggagagttttgggaaatgcatctgcctggatagataaggaaggagatccttgactattatgagtcattgggtgtgcaatagaagagccttggcttagcagcaatgagtctcataaagtggattttgtcatgacaagaggcagaaaggccaatgaggtagtctttaaaattaccatagccaagaaaagagagaaggagggcctgaactgcagaaggacagagggaattgctttctgctccctatcttggtttcttcatcttttaaaatttaaacctttcaactgtaatatttcataatgtgatttacacaggcaaagtaactagcatcatataacaaaactttatatatctatgcatatttactaagtaatatatatatttgtcatatatatatatttactatctatctacctatctatctatctactaagtaatgtgggtgaactttccagaccaccacctctctcagaactggattcttatataaatattttcccttttatcggaacccttcagtggtagttattatatagatggttggaattgggatgaccatttgaatttatccacacaactggaaaaagagtagaacaaatactttagccaactagtagtggtctgttgtttcactgaactagcaggttgatttagtgacattcatagaccaattgtgtggctgtgatggcggagtaaatgatggcagggaatattgggtgtgcatattgcgttgagtttttcctaaagcactaatggtaggaaccaagtaagtctttgtatattgtaggattatacagtcaagtggtggctcacgcctgtaatcccagcactttcagaggccaaggcaagtggatcacttgaggccaggatttcgagagcagcctggccgatgtgtgaaaccccgtctctactaaaaatgcaaaaattagctgggcatggtggtgcactcctgtaatcccagctacttgggaggctgaggcaggagaattgcttgaaccagggggttggaggttgcagtgagccgagatccatgcccctgcactcaagtctgggcaacagagtgagactctgtctcaaaaaacaaaacaaacaaacaaaagatgaaaagtaatgaaaactggaagttgtcagctctttgcaactcagttttgctcccagagacacactgttctaggtgatattaaatgttggttgcaggtaaacagacgtggaaagtgaagacctcctctgagtagggtggagaggttggtcacagcacaaagctttttctcagattcatgactcacttgtgtatggagatgtagacttagtgggcgggccaaattgttttctaaaaattaaatataaatctactgaaatcaaatctaactagattttaagggaaaaatagccaaatggtgttgcttatttccctctcgctgagagaatatggacacgtctagaagggcttttgagtttgttccctgagaccgttttttaaaaaacaataaccgaacctaagacttgaagctataaaaattctgtaagataacattgaaaaaacccttttagacattggtttagggaatgatttcatgaccaagaatccaaaagcaaatgcaatagaaacaaagagaaatagctgggagttaattaaactaaagagctttttgcacagcaaaatgaagtcagcagagtaaatagacaatccatggaatgggagaaaatcttcacaatctatacatctgatgaaaaacaaatatccagaatctacaacgaactcatacaaataagaaaaaaaacaaacaatctgatcaaaaagtgtactaaagacatgaatagacaattctcaaaagaagatatacaaatggccaacaaacatatgaaaaaatgctcaacatcactaatgatcagggaaatgcaaatcaaaaccacaatgtgataccaccttactcctgaaagaatggccataatccaagaatcaaaaaacagtagatggtgtggatgcagtgatcagggaacatttctacactgcaggtaggaatgtaaactagtacagccactatggaaaacagtgtggagattccttaaagaactgaaagtagaactaccatttgatccagcagtcccactactggctatctacccagaggaaaataagtcattattcgaaaaagatacttgcacacacatatttatagaagcacagttcacgattacaaagtcgtggaaccaacccaaaagcccatcaatcaatgagtggacaaagaaaccgtggtacatatatatgatggaatattactcagccataaaaaggaatgaattaacagcatttgcagtgacctggatgagattgaacactattattctaagtgaagtaactcaggaatggaaaaccaaacatcgtatgttctcactgatatgtgggagctaagctatgaggttgcaaaggcataagaatgatacaatggacattggggacttgggggaaagaatagggggtggcaagggataaaagacaacaaatatggtgcagtgtatactgcttaggtgatgggtgcaccaaaatctcaaaaattactacttaagaacttactcatataaccaaataccacctgtaccccaataacttatggaaaataaaataaaaatttaaaattaaaaaacaaacaaaagacaataaccagctgggtacagtggctcatgcctataatcccagcactttgggaggccaagacgggcagatcacctgaggtcaggagttcgagaccagtctggtcaacatggtgaaacactgcttctactaaatacacaaaaattagccgggcatggtggttggcatctgtaatcctaggtacttgggaggccgaggcaggagaatcacttgaacccgggagatggaggttgcagtgagccaagattgtgccattgtactccagcctgggcaacaagagcaagactctgtctcaaaaacaaaacaaaacaaaaaaaaacaaaccataaccctcctctttgcatgtcgtatacatgaggggagatacttgcttgagttttgcctcacactttagagcaaatttaaggctcatgatgatgagagaaatgtgagaaagcatttggtgtaccgcaaagtacaaagttccctcagtgtgtgggtttttctgtgagcggtaaaggtcttgggggaggctgtggtgtccaggtctcagtacttgcacagaagaacttgctatactgcagattttgttatgtcataagtcagagccatggaggcagcatgatgtcatgggaagagcaccgaaatgggagtcttgtctcacctttctcaggggataagggctaggtgagtaagccaatgagacttttaagcccaaacatcttcacagtattgtgttgcttacaaaaagaaagtggacaacaatatgaattatacacttctattaggttggtgcacaagtaatttcaccattacttttaatggcaaagactgcaattacttttgcaccaggctaatataaatcacagagtttgcttatgtggagaaatacttactgtcactaggagtcctagaacaataatttaacttcagttcatcttaatatgttgcttctgagaagagttgccagcctctgctacaccccagaaaaactaaaaaataagattgtgttaaaaatacgcttatcacctcatggttataatgtgatccagattggatttgaagtaattagagaataataggatggaatcaaattggtttctaaaatacatagcactagatcttctcaaattaagattctctggagtatttgaatcctatgagcattttttttcaatttttcattttcattttgatgctatctggacacaaactacctcaagcttagaaataatgactttctttcacagtgaaaattatcaatggcaaaacatttttatagaataagcatttgggttgttaagttcttggggatgttctgtgaagtgctaaagggatttaaattttcttctataaaaagagtccatgcaattccatctttgagaaacatttaatgtatcagatggcttttgctgtgtaacaaaccactctgaaacttactggctaaaccacaatcgtcattattacattttacaattttgtgggttggcaatttgggctgagatctgctcagtgtttttctggtcttggctggactcattcacatgtgtctgtggccagctactaggcctactaggaggctgtcataggatgagctcagcagggatggctctgcattgtctcacatcttccagcattctcatgaagtggcaggtctcctagagagtgagtggaagcatgcaaggcctcctgagatctaggcttagaatgggcatcctgtcacttctctcaattccactgatgaaagcaaatcataagttcaattcatatctaaggtggcagaacagaaccctccttcctttccctttctcccataatgttgcaaggaaatgaagataaagaggagatcattgcagccatttttgcagttgcaaacaatcagcaaaatgtatatgaaccagtgattttctaagtgtgcttaggaaaagccccttagggctgcgtggtgaagtgtgatgggtagggtgggtagacagtcaagggaggatttatgggcagtgacttggactctgtctcttttgcgtttaactaagcacctctggttccatctacttttgtttattttaaactattttgatttgaacaaaggtttccatacctaaaatgtcaaaaatcaagagtataacaaccgatgcaataggaagatggaaaggtgagagctccttgctagcagaagttgtgtttgggggaggggttgggggagcaagttgtgcagggagcattctggaagagtgtgacttgaacacaaatgagaaagaggattctctttgcgagtcagatccctgtgggtttctttctacatcccttactctgcctgacattagtgccttaaatataccaggtcctttataaagtgtttgataaaacagtgaatagagggatgaatgagcaacagtatacagggatcttaccatattttcctggagattaactccagtggagttaaacccacaattacttgcccgaagaagcactaaggaaatagctaaaatgaatggtctcacagatgtatagaaagctgaaaagagggaaacataaacagggacataacatagtgagtctgtctactgtatttatttatctaatctgtttgatcagaattattttgtgtaatatacacctagtaaaactgcagctattgaaagaaaaaagggaggattgaaacacctagcaatagatggaacattgttgttcacagacacacgaaacacaaatctgggctctgttttttaactcaggattcacatttttaagtacctttattagtttacttttaggccagggttggcattggaaattacctaaacacacttgatttgactgactttgaaaatgtgtattgaacccaagctagatagtatttttttttggttctggtttcaaatggggaaatatgcttaattatagcttgcatcatggcttggaaattatagcatttatatgttgatgtaacttgcctgaatgcaaaaagagggctatgtataggaaaagggacatatgtaaatagatccaaagcatgtttggcaaggcagaaaagctaaaagggaaagaggaaaatcgccttacctgccaaacatttagggaccacatgccaagtgttaccttcacttccttccaatctgtagccctgtttagtcttcagcctcatacataaaaccctctaaaattttagggtagaagtgggataacctctgagtgatttcagcacaattatttctggaaaaggtggacttgagtgtatggcagcttcctcagcatacattttaaataggaattgtacttaatttagatttttacattttgggatgaatgtgggtgatttctacagattttgacctctagctcaccttgtaaattgttttaattctcaaatgcagattcacatcaaagtagccaattgtattagttagctactgccatatgacattataaaatgaacaatcccaaactcagtgatttaaatcattgaaataaatgatttccatgaaagtttgtattgaccagtgtctactgatctaggcttggtactagctaggtggctggtatccaattgtcagctccagctgggcttggctgtgctagctctgcttcctgaatatttatcctggggccaaagatgagggggaaacaatacctggtggatggtcttctcatggcaaatgacaaggtgcaaaaaagtaagtgggaatacatgaggttgcttaaggcctatgctcagaaatggcattctaccagctctgctcacatgttatcagccaaagtttccaaggctaaggtgcaggcaagtgcactttgatgccaacctatgtccatcatcaccaactgccaatcaagaccaagatctttatgttgcaagtggtcatattccttaaaaacaagaaaaatgttttcacagagtgttctttaatatatggttggatgcaaaatattttggatgagaaaataagagactaagttgttttattattagttgtgtttgcattttacacactgcttgcactattatctgagtatgaattttaacaattgacttgcaatgagtaatgttcagaataatttatgtcaataaaatacttaatttacttttatacattttataatctcagcactttgggaggctgaggcaggcagatcacttgaggtcaggagttcgagaccagtctggccaacatgatgaaaccccgtctctactaaaaatacaaaaattagctaggcatggtggtgggcacctgtaatcctagctacttgggagactgaggcaggagaatcacttgaactctggagatggaggttgcagtgagccgagatcacaaaactgcactccagcctgggggacagagcaagaccctctctcaaaaaaaaacaaaaaagttatgaaagatcaagtgttttatgtttataattttttgatgacaatctttgaatataagaaacagaaaaaaagacaaccttgcactttaaaagaaaaataagctgctcatttccaaatttagttggtgttttagttactagcatttgtacctctcttctttgagaaggaaataattttaccatttaccccaaacctacttggcttctttcactctaagccctagctggatgtgtcagtggtttttggaggtggagttgaataacaattcagtgttaatagagtcacattattgaacttttctttccccagattgatgttctgccagaaattgtggaggctgtggaagggaaggtggaagtcttcctggacgggggtgtgcggaaaggcactgatgttctgaaagctctggctcttggcgccaaggctgtgtttgtggggagaccaatcgtttggggcttagctttccaggtaactggacaaagaaatgaatatataaaatagacaacttgacagtaaaacaaatgaataaaacaagtcagactgatttagttctgaatcactctgtatcttttcacttggttagggggagaaaggtgttcaagatgtcctcgagatactaaaggaagaattccggttggccatggctctgagtggtaagactcattcttgtttacaactttcttttcttttatgatctttaagtcaaggtccttggtggagagaagtgaatttgaaagggaagagtgtgggatcatttgagtacattaaatttgacgttgactccatatttacagctttgaggaactctgcatgtgcagtctctagtaattacttaacctctgtttttcacagctttattaggaatattttggtgagtacgagtacttggaggtggttgtaacatcatattcttcctcatctcataacacatgaacacatacatctacctggatagtcaacagctccctttggacatctaagtggcatcccaaactcttacctgtccaaaactaagctgctaactttccacgttaaacctgctccacctgcagtttctccatcagttcatgttaatcccatagtttcagttgctcagtacaaaaatcttcagtcattcttgactcttcttttctctcacaccttcttactcataggggctcgatcttcaaaatataatcagaatctgacctccctgtcatctccactgccaccgccctggctggagccatcattgtctctcatcccatggacattgtgggtgggcacgtctattctcacctatctcccctctcccagtctgttctgatcatggcggccagaacggttcttttaaacattcattgagatcatgttacttttctgatcagaaaacccccagtggcttttggtttactcagagtaaatgccagagtctttatagtggtttacaaggcctcatatggtttgactcccgtcatctttctgatcccaacttctaccaatgtcacccttacttattctgcccatgtgacattttgcctccatgatgtcccttgactatgtgagatatgtttcagccttggggtatttgcaatggttgttcgctctgcctggatgctcttcctccagacagctacatgaaaaacttctcatctttcatgtgttcacataaattgtactttcttaaaggtgcctatcctgattactgtgtttaaaatagcagtttatcctccacccatctattcgactccagttttcttttccagttttctttcatcctttttccatagtacctattgtctcctatatatcagctatcatagaatttccttttaaaattatatgcattatatattgtctgtttcccctttagaacataagatacatgaggcagggggctttgttgtcttgattgcagtattttcagtgtctacagcagtgcctggcacacaaacaatacatatttgctgaaagaataaataaataaatacacattaaaatactactagtgactatttctaatttgactcagaaagaacctgtagctagaaagaatggctgtgagttgattatcagctaccgcatttttgaaggtaaagagggcttccttttctgtaatccatatcatctggattgttcccatagataaaactccccagataaatggtctactcactactgtactcagtctgcagacagagcctcaacaagaagaatgccattctgtgaatcctcactatgcattttaatttgctacagacatatcctaaaagagagctttcagtcactgactctctattaaatgtggcagaaagaatatactctttgtgttaataaaaatatgtgccagtcattttgtgttaaggactgtttaggtaataaaaaatggtctcatgccacataagatttggcaagcctaccttgaatcataaaccttacatttgtcaaattttacattccttgggaaaacgattacctgcctgattattattgcattcagttcatattaaatgtatgcattattttttcagggtgccagaatgtgaaagtcatcgacaagacattggtgaggaaaaatcctttggccgtttccaagatctgacagtgcacaatattttcccatctgtattattttttttcagcatgtattacttgacaaagagacactgtgcagagggtgaccacagtctgtaattccccacttcaatacaaagggtgtcgttcttttccaacaaaatagcaatcccttttatttcattgcttttgacttttcaatgggtgtcctaggaaccttttagaaagaaatggactttcatcctggaaatatattaactgttaaaaagaaaacattgaaaatgtgtttagacaacgtcatcccctggcaggctaaagtgctgtatcctttagtaaaattggaggtagcaaacactaaggtgaaaagataatgatctcattgtttattaacctgtattctgtttacatgtctttaaaacagtggttcttaaattgtaagctcaggttcaaagtgttggtaatgcctgattcacaactttgagaaggtagcactggagagaattggaatgggtggcggtaattggtgatacttctttgaatgtagatttccaatcacatctttagtgtctgaatatatccaaatgttttaggatgtatgttacttcttagagagaaataaagcatttttgggaagaat

SEQ ID NO: 4 - DNA, 71 bp, Homo sapiens
ggtgccagaatgtgaaagtcatcgacaagacattggtgaggaaaaatcctttggccgtttccaagatctga

SEQ ID NO: 5 - DNA, 22 bp, Homo sapiens
aagacattggtgaggaaaaatc

SEQ ID NO: 6 - DNA, 22 bp, Homo sapiens
ttctgtaaccactcctttttag

SEQ ID NO: 7 - protein, 354 aa, Artificial Sequence
  Synthesized
MNTKYNKEFLLYLAGFVDGDGSIYAAIRPSQTAKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVRQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 8 - protein, 354 aa, Artificial Sequence
  Synthesized
MNTKYNKEFLLYLAGFVDGDGSIYAAIRPSQTCKFKHRLQLFFAVMQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVKQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 9 - protein, 354 aa, Artificial Sequence
  Synthesized
MNTKYNKEFLLYLAGFVDGDGSIYAAIRPSQSCKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVRQRTRRRWFLDKLVDEIGVGYVIDEGSVSSYRLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 10 - protein, 354 aa, Artificial Sequence
  Synthesized
MNTKYNKEFLLYLAGFVDGDGSIYAAIRPSQNKKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVRQHTRRRWFLDKLVDEIGVGYVIDESKTASYRLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 11 - protein, 147 aa, Artificial Sequence
  Synthesized
KEFLLYLAGFVDGDGSIYAAIRPSQTAKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLD

SEQ ID NO: 12 - protein, 147 aa, Artificial Sequence
  Synthesized
KEFLLYLAGFVDGDGSIYAAIRPSQTCKFKHRLQLFFAVMQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLD

SEQ ID NO: 13 - protein, 147 aa, Artificial Sequence
  Synthesized
KEFLLYLAGFVDGDGSIYAAIRPSQSCKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLD

SEQ ID NO: 14 - protein, 147 aa, Artificial Sequence
  Synthesized
KEFLLYLAGFVDGDGSIYAAIRPSQNKKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLD

SEQ ID NO: 15 - protein, 147 aa, Artificial Sequence
  Synthesized
KEFLLYLAGFVDGDGSIFASIHPQQRNKFKHQLSLHFTVRQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLD

SEQ ID NO: 16 - protein, 147 aa, Artificial Sequence
  Synthesized
KEFLLYLAGFVDGDGSIFASIHPQQRNKFKHQLSLHFTVKQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLD

SEQ ID NO: 17 - protein, 147 aa, Artificial Sequence
  Synthesized
KEFLLYLAGFVDGDGSIFASIHPQQRNKFKHQLSLHFTVRQRTRRRWFLDKLVDEIGVGYVIDEGSVSSYRLSEIKPLHNFLTQLQPFLKLKQKQANLVLKIIEQLPSAKESPDKFLEVCTWVDQIAALNDSRTRKTTSETVRAVLD

SEQ ID NO: 18 - protein, 147 aa, Artificial Sequence
  Synthesized
KEFLLYLAGFVDGDGSIFASIHPQQRNKFKHQLSLHFTVRQHTRRRWFLDKLVDEIGVGYVIDESKTASYRLSQIKPLHNFLTQLQPFLKLKQKQANLVLKIIEQLPSAKESPDKFLEVCTWVDQIAALNDSRTRKTTSETVRAVLD

SEQ ID NO: 19 - protein, 347 aa, Homo sapiens
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALS

SEQ ID NO: 20 - protein, 347 aa, Macaca mulatta
MLPRLICINDYEQHAKSVLPKSIYDYYRSGANDEETLADNVAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVQQAEKTGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIIWGLAFQGEKGVQDVLEILKEEFRLAMALS

SEQ ID NO: 21 - protein, 347 aa, Mus musculus
MLPRLVCISDYEQHVRSVLQKSVYDYYRSGANDQETLADNIQAFSRWKLYPRMLRNVADIDLSTSVLGQRVSMPICVGATAMQCMAHVDGELATVRACQTMGTGMMLSSWATSSIEEVAEAGPEALRWMQLYIYKDREISRQIVKRAEKQGYKAIFVTVDTPYLGNRIDDVRNRFKLPPQLRMKNFETNDLAFSPKGNFGDNSGLAEYVAQAIDPSLSWDDITWLRRLTSLPIVVKGILRGDDAKEAVKHGVDGILVSNHGARQLDGVPATIDVLPEIVEAVEGKVEVFLDGGVRKGTDVLKALALGAKAVFVGRPIIWGLAFQGEKGVQDVLEILKEEFRLAMALS

SEQ ID NO: 22 - protein, 367 aa, Artificial Sequence
  Synthesized
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALSGCQNVKVIDKTLVRKNPLAV

SEQ ID NO: 23 - DNA, 22 bp, Homo sapiens
aagtcatcgacaagacattggt

SEQ ID NO: 24 - DNA, 22 bp, Homo sapiens
atattaaatgtatgcattattt

SEQ ID NO: 25 - DNA, 6 bp, Homo sapiens
tttttc

SEQ ID NO: 26 - DNA, 6 bp, Homo sapiens
ttcagg

SEQ ID NO: 27 - DNA, 6 bp, Homo sapiens
gggtgc

SEQ ID NO: 28 - DNA, 6 bp, Homo sapiens
ggtgcc

SEQ ID NO: 29 - DNA, 6 bp, Homo sapiens
tgccag

SEQ ID NO: 30 - DNA, 6 bp, Homo sapiens
gccaga

SEQ ID NO: 31 - DNA, 6 bp, Homo sapiens
cagaat

SEQ ID NO: 32 - DNA, 6 bp, Homo sapiens
gaatgt

SEQ ID NO: 33 - DNA, 6 bp, Homo sapiens
acaaga

SEQ ID NO: 34 - DNA, 6 bp, Homo sapiens
acattg

SEQ ID NO: 35 - DNA, 6 bp, Homo sapiens
cgtttc

SEQ ID NO: 36 - DNA, 6 bp, Homo sapiens
ttccaa

SEQ ID NO: 37 - DNA, 6 bp, Homo sapiens
caagat

SEQ ID NO: 38 - DNA, 6 bp, Homo sapiens
agatct

SEQ ID NO: 39 - DNA, 6 bp, Homo sapiens
agatct

SEQ ID NO: 40 - DNA, 6 bp, Homo sapiens
atcttg

SEQ ID NO: 41 - DNA, 6 bp, Homo sapiens
ttggaa

SEQ ID NO: 42 - DNA, 6 bp, Homo sapiens
gaaacg

SEQ ID NO: 43 - DNA, 6 bp, Homo sapiens
caatgt

SEQ ID NO: 44 - DNA, 6 bp, Homo sapiens
tcttgt

SEQ ID NO: 45 - DNA, 6 bp, Homo sapiens
acattc

SEQ ID NO: 46 - DNA, 6 bp, Homo sapiens
attctg

SEQ ID NO: 47 - DNA, 6 bp, Homo sapiens
tctggc

SEQ ID NO: 48 - DNA, 6 bp, Homo sapiens
ctggca

SEQ ID NO: 49 - DNA, 6 bp, Homo sapiens
ggcacc

SEQ ID NO: 50 - DNA, 6 bp, Homo sapiens
gcaccc

SEQ ID NO: 51 - DNA, 6 bp, Homo sapiens
cctgaa

SEQ ID NO: 52 - DNA, 6 bp, Homo sapiens
gaaaaa

SEQ ID NO: 53 - DNA, 20 bp, Homo sapiens
ggaaaaatcctttggccgtt

SEQ ID NO: 54 - DNA, 18 bp, Homo sapiens
ttggtgaggaaaaatcct

SEQ ID NO: 55 - DNA, 20 bp, Homo sapiens
cattggtgaggaaaaatcct

SEQ ID NO: 56 - DNA, 18 bp, Homo sapiens
gacattggtgaggaaaaa

SEQ ID NO: 57 - DNA, 19 bp, Homo sapiens
ggaaaaatcctttggccgt

SEQ ID NO: 58 - DNA, 16 bp, Homo sapiens
cattggtgaggaaaaa

SEQ ID NO: 59 - DNA, 19 bp, Homo sapiens
tgccagaatgtgaaagtca

SEQ ID NO: 60 - DNA, 16 bp, Homo sapiens
tgccagaatgtgaaag

SEQ ID NO: 61 - DNA, 19 bp, Homo sapiens
gggtgccagaatgtgaaag

SEQ ID NO: 62 - DNA, 18 bp, Homo sapiens
agatctgacagtgcacaa

SEQ ID NO: 63 - DNA, 17 bp, Homo sapiens
gatctgacagtgcacaa

SEQ ID NO: 64 - DNA, 15 bp, Homo sapiens
tctgacagtgcacaa

SEQ ID NO: 65 - DNA, 17 bp, Homo sapiens
aaaatcctttggccgtt

SEQ ID NO: 66 - DNA, 16 bp, Homo sapiens
aaatcctttggccgtt

SEQ ID NO: 67 - DNA, 15 bp, Homo sapiens
aatcctttggccgtt

SEQ ID NO: 68 - DNA, 15 bp, Homo sapiens
tggccgtttccaaga

SEQ ID NO: 69 - DNA, 20 bp, Homo sapiens
agtcatcgacaagacattgg

SEQ ID NO: 70 - DNA, 19 bp, Homo sapiens
gtcatcgacaagacattgg

SEQ ID NO: 71 - DNA, 15 bp, Homo sapiens
tcgacaagacattgg

SEQ ID NO: 72 - DNA, 17 bp, Homo sapiens
caaaggatttttcctca

SEQ ID NO: 73 - DNA, 16 bp, Homo sapiens
gccaaaggatttttcc

SEQ ID NO: 74 - DNA, 17 bp, Homo sapiens
ttttcctcaccaatgtc

SEQ ID NO: 75 - DNA, 18 bp, Homo sapiens
tttttcctcaccaatgtc

SEQ ID NO: 76 - DNA, 20 bp, Homo sapiens
gatttttcctcaccaatgtc

SEQ ID NO: 77 - DNA, 19 bp, Homo sapiens
tgactttcacattctggca

SEQ ID NO: 78 - DNA, 17 bp, Homo sapiens
ttctggcaccctgaaaa

SEQ ID NO: 79 - DNA, 19 bp, Homo sapiens
ttctggcaccctgaaaaaa

SEQ ID NO: 80 - DNA, 16 bp, Homo sapiens
cacattctggcaccct

SEQ ID NO: 81 - DNA, 16 bp, Homo sapiens
tggcaccctgaaaaaa

SEQ ID NO: 82 - DNA, 15 bp, Homo sapiens
gtgcactgtcagatc

SEQ ID NO: 83 - DNA, 18 bp, Homo sapiens
attgtgcactgtcagatc

SEQ ID NO: 84 - DNA, 16 bp, Homo sapiens
tctggcaccctgaaaa

SEQ ID NO: 85 - DNA, 19 bp, Homo sapiens
cattctggcaccctgaaaa

SEQ ID NO: 86 - DNA, 17 bp, Homo sapiens
cgatgactttcacattc

SEQ ID NO: 87 - DNA, 18 bp, Homo sapiens
tcgatgactttcacattc

SEQ ID NO: 88 - DNA, 20 bp, Homo sapiens
tgtcgatgactttcacattc

SEQ ID NO: 89 - DNA, 19 bp, Homo sapiens
gtcgatgactttcacattc

SEQ ID NO: 90 - DNA, 15 bp, Homo sapiens
cgatgactttcacat

SEQ ID NO: 91 - DNA, 15 bp, Homo sapiens
ttgtcgatgactttc

SEQ ID NO: 92 - DNA, 15 bp, Homo sapiens
tgtcgatgactttca

SEQ ID NO: 93 - DNA, 16 bp, Homo sapiens
tgtcgatgactttcac

SEQ ID NO: 94 - DNA, 15 bp, Homo sapiens
gtcgatgactttcac

SEQ ID NO: 95 - DNA, 15 bp, Homo sapiens
tcgatgactttcaca

SEQ ID NO: 96 - DNA, 17 bp, Homo sapiens
ttgtcgatgactttcac

SEQ ID NO: 97 - DNA, 23 bp, Homo sapiens
tcatcgacaagacattggtgagg

SEQ ID NO: 98 - DNA, 23 bp, Homo sapiens
gaaagtcatcgacaagacattgg

SEQ ID NO: 99 - DNA, 23 bp, Homo sapiens
attggtgaggaaaaatcctttgg

SEQ ID NO: 100 - DNA, 23 bp, Homo sapiens
atgtatgcattattttttcaggg

SEQ ID NO: 101 - DNA, 23 bp, Homo sapiens
aatgtatgcattattttttcagg

SEQ ID NO: 102 - DNA, 23 bp, Homo sapiens
tgtcgatgactttcacattctgg

SEQ ID NO: 103 - DNA, 23 bp, Homo sapiens
cagatcttggaaacggccaaagg

SEQ ID NO: 104 - DNA, 23 bp, Homo sapiens
gcactgtcagatcttggaaacgg

SEQ ID NO: 105 - DNA, 23 bp, Homo sapiens
tattgtgcactgtcagatcttgg

SEQ ID NO: 106 - DNA, 28 bp, Homo sapiens
ttttttcagggtgccagaatgtgaaagt

SEQ ID NO: 107 - DNA, 28 bp, Homo sapiens
tttttcagggtgccagaatgtgaaagtc

SEQ ID NO: 108 - DNA, 28 bp, Homo sapiens
ttttcagggtgccagaatgtgaaagtca

SEQ ID NO: 109 - DNA, 28 bp, Homo sapiens
tttggccgtttccaagatctgacagtgc

SEQ ID NO: 110 - DNA, 28 bp, Homo sapiens
tttcagggtgccagaatgtgaaagtcat

SEQ ID NO: 111 - DNA, 28 bp, Homo sapiens
tttccaagatctgacagtgcacaatatt

SEQ ID NO: 112 - DNA, 28 bp, Homo sapiens
tttcctcaccaatgtcttgtcgatgact

SEQ ID NO: 113 - DNA, 28 bp, Homo sapiens
ttttcctcaccaatgtcttgtcgatgac

SEQ ID NO: 114 - DNA, 28 bp, Homo sapiens
tttttcctcaccaatgtcttgtcgatga

SEQ ID NO: 115 - DNA, 28 bp, Homo sapiens
tttcacattctggcaccctgaaaaaata

SEQ ID NO: 116 - DNA, 19 bp, Homo sapiens
ggtgccagaatgtgaaagt

SEQ ID NO: 117 - DNA, 17 bp, Homo sapiens
tggtcaccctctgcaca

SEQ ID NO: 118 - DNA, 26 bp, Homo sapiens
gacattggtgaggaaaaatcctttgg

SEQ ID NO: 119 - DNA, 17 bp, Homo sapiens
gtgatgatgccagggag

SEQ ID NO: 120 - DNA, 17 bp, Homo sapiens
ccatcgagttgtcgagc

SEQ ID NO: 121 - DNA, 24 bp, Homo sapiens
gaatgggatcttggtgtcgaatca

SEQ ID NO: 122 - DNA, 19 bp, Homo sapiens
gtgatgatgccaaggaagc

SEQ ID NO: 123 - DNA, 17 bp, Homo sapiens
gtagctggcaccccatc

SEQ ID NO: 124 - DNA, 20 bp, Homo sapiens
tgggatcttggtgtcgaatc

SEQ ID NO: 125 - DNA, 22 bp, Homo sapiens
ccttgggaaaacgattacctgc

SEQ ID NO: 126 - DNA, 22 bp, Homo sapiens
gagttacagtctgtggtcaccc

SEQ ID NO: 127 - DNA, 33 bp, Artificial Sequence
  Synthesized
sgsvggssassaassasssgsgsaragagsgtg

SEQ ID NO: 128 - protein, 354 aa, Artificial Sequence
  Synthesized
MNTKYNKEFLLYLAGFVDSDGSIYAAIRPSQTAKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVRQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLDSLSEKKKSSP